(19) United States
(12) Patent Application Publication
(10) Pub. No.: US 2021/0071256 A1
(43) Pub. Date: Mar. 11, 2021

(54) SYSTEMS AND METHODS FOR PAIRWISE INFERENCE OF DRUG-GENE INTERACTION NETWORKS

(71) Applicant: Recursion Pharmaceuticals, Inc., Salt Lake City, UT (US)

(72) Inventors: Ian QUIGLEY, Salt Lake City, UT (US); Emery GOOSSENS, Salt Lake City, UT (US)

(73) Assignee: Recursion Pharmaceuticals, Inc., Salt Lake City, UT (US)

(21) Appl. No.: 17/017,298

(22) Filed: Sep. 10, 2020

Related U.S. Application Data
(60) Provisional application No. 62/899,006, filed on Sep. 11, 2019.

(51) Int. Cl. C12Q 1/6883; G16B 40/00; G16B 25/00; G06N 3/02

(52) U.S. Cl. CPC C12Q 1/6883; G16B 40/00; C12Q 2600/158; G06N 3/02; G16B 25/00

(57) ABSTRACT

Methods and systems are provided for determining whether a first cellular perturbation interacts with a second cellular perturbation in one of a specific cellular context and a background, in a cell based assay. Data points for one or more baseline state, perturbation state, compound state, and combination state are obtained, where the data points each include data for a plurality of cellular characteristics acquired across instances of the respective cellular state. A dimension reduction model is applied the data points to achieve a plurality of feature values from each of the data points. It is then determined whether the first cellular perturbation interacts with the second cellular perturbation in one of a specific cellular context and a background by using the features values achieved from the data points to resolve whether the combination of the gene and the compound has a threshold interaction effect on one or more cellular characteristics.

### CROSS REFERENCE TO RELATED APPLICATIONS

This application claims priority to and benefit of co-pending U.S. Provisional Patent Application No. 62/899,006 filed on Sep. 11, 2019 entitled “SYSTEMS AND METHODS FOR PAIRWISE INFERENCE OF DRUG-GENE INTERACTION NETWORKS” by Ian Quigley et al., having Attorney Docket No. R2018-5009-PR, and assigned to the assignee of the present application, the disclosure of which is hereby incorporated herein by reference in its entirety.

### BACKGROUND

High throughput screening (HTS) is a process used in pharmaceutical drug discovery to test large compound libraries containing thousands to millions of compounds for various biological effects. HTS typically uses robotics, such as liquid handlers and automated imaging devices, to conduct tens of thousands to tens of millions of assays, e.g., biochemical, genetic, and/or phenotypical, on the large compound libraries in multi-well plates, e.g., 96-well, 384-well, 1536-well, or 3456-well plates. In this fashion, lead-compounds that provide a desired biochemical, genetic, or phenotypic effect can be quickly identified from the large compound libraries, for further testing and development towards the goal of discovering a new pharmaceutical agent for disease treatment. For a review of basic HTS methodologies see, for example, Wildey et al., 2017, “Chapter Five—High-Throughput Screening,” Annual Reports in Medicinal Chemistry, Academic Press, 50:149-95, which is hereby incorporated by reference.

However, while HTS facilitates identification of candidate compounds that providing a particular effect in an assay, it does not provide information about the mechanism of action of the candidate compound, whether the compound may have off-target effects, or what biological agents the compound may interact with in vivo. Thus, significant time and effort is wasted in the pharmaceutical industry pursuing non-viable candidate compounds that could have been eliminated from consideration earlier in the process, had this information been available.

In theory, some of this information could be elucidated at an earlier stage of the drug discovery process, if comprehensive biological interaction networks were available. However, conventional methodologies for identifying biological interactions, e.g., interactions between genes, compounds, soluble factors, toxins, etc., are inefficient. For example, a classic interaction discovery method, synthetic lethality (see, Nijman S M., FEBS Lett., 585(1):1-6 (2011) for review), uses genetic perturbations to identify drug activity or genetic interactions. Briefly, two genes (or a gene and a compound) are thought to interact in some biologically meaningful way if the individual perturbations don't kill the cell but the pair does. This approach has been used to map genetic interactions in yeast (see, for example, Costanzo M, et al., Science, 353(6306) pii:aaf1420 (2016)) and human cells (see, for example, Horlbeck Mass., et al., Cell, 174(4):953-67 (2018)) and identify protein targets of drugs (see, for example, Jost M, et al., Mol Cell, 68(1):210-23 (2017)). However, these methodologies rely on single-dimensional readouts, namely cell survival or impaired growth. This limits the reach of the approach to genes and compounds that significantly affect cell survival or growth. Similarly, other approaches used in new chemical entity screening center on non-scalable methods, such as affinity purification and mass spectrometry approaches or structural similarity to compounds with known mechanisms-of-action. These approaches are inefficient and limit benchmarking to well-documented compound activities.

Existing methods that are capable of facilitating elucidation of some of this information at an earlier stage in the drug discovery process suffer from several short falls.

### SUMMARY

Given the above background, what is needed in the art are improved systems and methods for identifying interactions between agents in a complex biological system. The present disclosure addresses, among others, the need for systems and methods for identifying interactions within complex biological systems using a cell-based assay. Advantageously, the systems and methods described herein are able to identify interactions in a high-throughput fashion, and without being limited to a phenotypic read-out linked to cell death or cellular growth abnormalities. Further, in some embodiments, the systems and methods described herein facilitate identification of the mechanism of action for a compound, e.g., by comparing high-dimensional featurized vectors derived from cellular characteristics. In yet other embodiments, the methods and systems described herein facilitate identification of polypharmacological effects test compounds.

The methods and systems disclosed herein leverage automated biology and artificial intelligence. In some embodiments, the use of microscopy to measure hundreds of sub-cellular structural changes caused by pathogenic perturbations facilitates discovery of data-rich “marker-less” high-dimensional phenotypes in vitro. High-throughput screens on these phenotypes uncovers interactions between biological agents, e.g., genes, drug compounds, soluble factors, and toxins, which cannot be identified using conventional synthetic lethality approaches. Moreover, interactions that are not mediated by a physical interaction between the biological agents can also be uncovered, which is not the case for conventional techniques that rely on the detection of physical interactions. This unique approach allows rapid modeling and screening of interactions between many different types of biological agents in a complex biological environment.

In one aspect, the disclosure provides methods, systems, and computable readable media for determining whether a compound interacts with a gene, in a cell based assay. The cell based assay includes a plurality of wells across one or more plates. The method includes obtaining a baseline data point for a baseline state, where the baseline data point includes a plurality of dimensions, each respective dimension in the plurality of dimensions of the baseline data point representing a corresponding measure of central tendency of a different cellular characteristic, in a plurality of cellular characteristics, determined across a plurality of baseline aliquots of cells representing the baseline state in corresponding wells, in the plurality of wells, where the baseline state includes a first cellular context. The method also includes obtaining a perturbation data point for a perturbation state, where the perturbation data point includes the plurality of dimensions, each respective dimension in the plurality of dimensions of the perturbation data point representing the measurement of central tendency of a different cellular characteristic, in the plurality of cellular characteristics, determined across a plurality of perturbation aliquots of cells representing the perturbation state in corresponding wells, in the plurality of wells, where the perturbation state includes a first perturbation of the first cellular context in which the expression of a gene is perturbed relative to the expression of the gene in the baseline state. The method also includes obtaining a compound data point for a compound state, where the compound data point includes the plurality of dimensions, each respective dimension in the plurality of dimensions of the compound data point representing the measurement of central tendency of a different cellular characteristic, in the plurality of cellular characteristics, determined across a plurality of compound aliquots of cells representing the compound state in corresponding wells, in the plurality of wells, where the compound state includes a second perturbation of the first cellular context in which the first cellular context is exposed to a compound. The method also includes obtaining a combination data point for a combination state, where the combination data point includes the plurality of dimensions, each respective dimension in the plurality of dimensions of the combination data point representing the measurement of central tendency of a different cellular characteristic, in the plurality of cellular characteristics, determined across a corresponding plurality of combination aliquots of cells representing the combination state in corresponding wells, in the plurality of wells, where the combination state includes a third perturbation of the first cellular context in which (i) the expression of the gene is perturbed relative to the expression of the gene in the baseline state and (ii) the first cellular context is exposed to the compound. The method then includes featurizing the baseline data point by applying a dimension reduction model to the baseline data point, thereby generating a plurality of baseline feature values for the baseline data point, featurizing the perturbation data point by applying the dimension reduction model to the perturbation data point, thereby generating a plurality of perturbation feature values for the perturbation data point, featurizing the compound data point by applying the dimension reduction model to the compound data point, thereby generating a plurality of compound feature values for the compound data point, and featurizing the combination data point by applying the dimension reduction model to the combination data point, thereby generating a plurality of combination feature values for the combination data point. The method then includes determining whether the compound interacts with the gene by using the plurality of baseline feature values, the plurality of perturbation feature values, the plurality of compound feature values, and the plurality of combination feature values to resolve whether the combination of the gene and the compound has a threshold interaction effect on one or more cellular characteristic in the plurality of cellular characteristics. The compound interacts with the gene when the combination of the gene and the compound has a threshold effect on one or more cellular characteristic in the plurality of cellular characteristics. The compound does not interact with the gene when the combination of the gene and the compound does not have a threshold effect on one or more cellular characteristic in the plurality of cellular characteristics.

In another aspect, the disclosure provides methods, systems, and computable readable media for determining whether two compounds affect a cell through a common or redundant pathway, in a cell based assay. The cell based assay including a plurality of wells across one or more plates. The method includes obtaining a baseline data point for a baseline state, where the baseline data point includes a plurality of dimensions, each respective dimension in the plurality of dimensions of the baseline data point representing a corresponding measure of central tendency of a different cellular characteristic, in a plurality of cellular characteristics, determined across a plurality of baseline aliquots of cells representing the baseline state in corresponding wells, in the plurality of wells, where the baseline state includes a first cellular context. The method also includes obtaining a first compound data point for a first compound state, where the first compound data point includes the plurality of dimensions, each respective dimension in the plurality of dimensions of the first compound data point representing the measurement of central tendency of a different cellular characteristic, in the plurality of cellular characteristics, determined across a plurality of first compound aliquots of cells representing the first compound state in corresponding wells, in the plurality of wells, where the first compound state includes a first perturbation of the first cellular context in which the first cellular context is exposed to a first compound. The method also includes obtaining a second compound data point for a second compound state, where the second compound data point includes the plurality of dimensions, each respective dimension in the plurality of dimensions of the second compound data point representing the measurement of central tendency of a different cellular characteristic, in the plurality of cellular characteristics, determined across a plurality of second compound aliquots of cells representing the second compound state in corresponding wells, in the plurality of wells, where the second compound state includes a second perturbation of the first cellular context in which the first cellular context is exposed to a second compound. The method also includes obtaining a combination data point for a combination state, where the combination data point includes the plurality of dimensions, each respective dimension in the plurality of dimensions of the combination data point representing the measurement of central tendency of a different cellular characteristic, in the plurality of cellular characteristics, determined across a corresponding plurality of combination aliquots of cells representing the combination state in corresponding wells, in the plurality of wells, where the combination state includes a third perturbation of the first cellular context in which the first cellular context is exposed to both the first compound and the second compound. The method then includes featurizing the baseline data point by applying a dimension reduction model to the baseline data point, thereby generating a plurality of baseline feature values for the baseline data point, featurizing the first compound data point by applying a dimension reduction model to the first compound data point, thereby generating a plurality of first compound feature values for the first compound data point, featurizing the second compound data point by applying the dimension reduction model to the second compound data point, thereby generating a plurality of second compound feature values for the second compound data point, and featurizing the combination data point by applying the dimension reduction model to the combination data point, thereby generating a plurality of combination feature values for the combination data point. The method then includes determining whether the first compound and the second compound affect the cell through a common or redundant pathway by using the plurality of baseline feature values, the plurality of first compound feature values, the plurality of second compound feature values, and the plurality of combination feature values to resolve whether the combination of the first compound and the second compound satisfy a threshold interaction criterion involving one or more cellular characteristic in the plurality of cellular characteristics. The first compound and the second compound affect the cell through a common or redundant pathway when the combination of the first compound and the second compound satisfy the threshold interaction effect. The first compound and the second compound do not affect the cell through a common or redundant pathway when the combination of the first compound and the second compound does not satisfy the threshold interaction effect.

In another aspect, the disclosure provides methods, systems, and computable readable media for determining whether a first cellular perturbation interacts with a second cellular perturbation in one of a specific cellular context and a background, in a cell based assay, the cell based assay comprising a plurality of wells across one or more plates, the computer system comprises: one or more processors; a memory; and one or more programs, wherein the one or more programs are stored in the memory and are configured to be executed by the one or more processors, the one or more programs including instructions for: obtaining a baseline data point for a baseline state, wherein the baseline data point comprises a plurality of dimensions, in a plurality of cellular characteristics, determined across a plurality of baseline aliquots of cells representing the baseline state in corresponding wells, in the plurality of wells, wherein the baseline state comprises a first cellular context; obtaining a perturbation data point for a perturbation state, wherein the perturbation data point comprises the plurality of dimensions, in the plurality of cellular characteristics, determined across a plurality of perturbation aliquots of cells representing the perturbation state in corresponding wells, in the plurality of wells, wherein the perturbation state comprises a first perturbation of the first cellular context in which expression of a gene is perturbed relative to expression of the gene in the baseline state; obtaining a compound data point for a compound state, wherein the compound data point comprises the plurality of dimensions, in the plurality of cellular characteristics, determined across a plurality of compound aliquots of cells representing the compound state in corresponding wells, in the plurality of wells, wherein the compound state comprises a second perturbation of the first cellular context in which the first cellular context is exposed to a compound; obtaining a combination data point for a combination state, wherein the combination data point comprises the plurality of dimensions, in the plurality of cellular characteristics, determined across a corresponding plurality of combination aliquots of cells representing the combination state in corresponding wells, in the plurality of wells, wherein the combination state comprises a third perturbation of the first cellular context in which (i) expression of the gene is perturbed relative to expression of the gene in the baseline state and (ii) the first cellular context is exposed to the compound; applying a dimension reduction model, in turn, to each of the baseline data point, the perturbation data point, the compound data point, and the combination data point to respectively generate a plurality of baseline feature values for the baseline data point, a plurality of perturbation features values for the perturbation data point, a plurality of compound feature values for the compound data point, and a plurality of combination feature values for the combination data point; and determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics.

### BRIEF DESCRIPTION OF THE DRAWINGS

FIGS. 1A and 1B collectively illustrate an exemplary workflow for identifying interactions within complex biological systems, in accordance with various embodiments of the present disclosure.

FIGS. 2A, 2B, 2C, 2D, 2E, and 2F collectively illustrate a device for identifying interactions within complex biological systems, in accordance with various embodiments of the present disclosure.

FIGS. 3A-3D illustrate an example process for obtaining data using a high-throughput cell-based assay, in accordance with various embodiments of the present disclosure.

FIGS. 4A, 4B, 4C, and 4D collectively illustrate an example process for identifying an interaction between a compound and a gene in a complex biological system, in accordance with various embodiments of the present disclosure.

FIGS. 5A, 5B, 5C, 5D, 5E, 5F, 5G and 5H collectively illustrate an example process for identifying interactions between compounds and genes in a complex biological system, in accordance with various embodiments of the present disclosure.

FIGS. 6A, 6B, 6C, and 6D collectively illustrate an example process for determining whether two compounds affect a cell through a common or redundant pathway, in accordance with various embodiments of the present disclosure.

FIGS. 7A, 7B, 7C, 7D, 7E, 7F and 7G collectively illustrate an example process for identifying compounds that affect a cell through a common or redundant pathway, in accordance with various embodiments of the present disclosure.

FIGS. 8A, 8B, 8C, and 8D collectively illustrate an example process for determining whether a cellular perturbation interacts with a second cellular perturbation in one of a specific cellular context and background, in accordance with various embodiments of the present disclosure.

FIG. 9 illustrates an example neural network having utility as a dimension reduction model, in accordance with various embodiments of the present disclosure.

FIG. 10 shows a rug plot of the combined p-value test statistic for interactions between known JAK inhibitors or unannotated compounds and a perturbation in IL13 gene expression, in accordance with some embodiments of the present disclosure.

Like reference numerals refer to corresponding parts throughout the several views of the drawings.

### DESCRIPTION OF EMBODIMENTS

### Overview

Modeling of large biological interaction networks holds great promise for improving drug discovery, particularly in the field of new chemical entity screening. Advantageously, the present disclosure provides improved methods and systems for efficiently identifying biological interactions that do not suffer from the same drawbacks as conventional methods for identifying biological interactions.

For instance, in some embodiments, the methods and systems provided herein facilitate linking compound effects to particular genes or pathways in a cell, by perturbing genes singly and in combination with the compound. Rather than counting cells or looking for changes in the growth rate of the altered cells, the systems and methods herein determine interactions in an unbiased fashion through acquisition of a high-dimensional suite of image features, preferably in a high-throughput fashion. From the information provided in these high-throughput screens, complex compound-gene, compound-compound and gene-gene interaction networks can be built, which will provide insight into how candidate drug compounds, and particularly new chemical entities are interacting with the proteome of a cell.

In some embodiments, the methods and systems provided herein allow building of gene-gene interaction networks, and the probing of compounds of interest (e.g. lead compounds) against panels of critical genes, in order to understand what the compound is doing in cells. Those ‘critical genes’ can be picked by selecting sparsely from the gene-gene networks, or by using subsets of genes/proteins, such as specific pathways or the druggable genome. In some embodiments, the systems and methods described herein also allow identification of the mechanism of action of a compound, e.g., from a single drug screen.

### Definitions

The terminology used in the present disclosure is for the purpose of describing particular embodiments only and is not intended to be limiting. As used in the description of and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It will also be understood that, although the terms first, second, etc. may be used herein to describe various elements, these elements should not be limited by these terms. These terms are only used to distinguish one element from another. For example, a first compound could be termed a second compound and, similarly, a second compound could be termed a first compound, without departing from the scope of the present disclosure. The first compound and the second compound are both compounds, but they are not the same compound. Furthermore, the terms “subject,” “user,” and “patient” are used interchangeably herein.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, an experimental “state,” as in a “baseline state,” “perturbation state,” “compound state,” or “combination state,” refers to an experimental condition including an aliquot of cells of one or more cellular contexts, which may or may not be perturbed relative to a reference cellular context, and a chemical environment, e.g., a culture medium, which may or may not include a test compound. In some embodiments, an experimental state is imaged using one or more cellular dyes that are added to the experimental state after passage of a sufficient assay time that allows for changes in cellular morphology in the experimental state, relative to a reference state, e.g., via cell painting. Further details regarding methodologies for measuring cellular characteristics in an experimental state, both visually and non-visually, are described herein below.

As used herein, a “baseline state,” refers to a reference experimental condition that includes an aliquot of a reference cellular context and a reference chemical environment. Measurements of characteristics of the reference cellular context in the baseline state are used as a comparison to measurements of cellular characteristics acquired from other experimental states, e.g., perturbation states, compound states, and combination states, in order to identify differences in the cellular characteristics of the other experimental states caused by a change in the experimental conditions, e.g., gene expression perturbation and/or exposure to a test compound. In some embodiments, the baseline state represents the average of a plurality of reference experimental conditions, e.g., as measured across a plurality of baseline wells in one or more multiwell plate. In some embodiments, each of the respective reference experimental conditions across which the baseline state is averaged have the same composition, e.g., the same reference cellular context and the same reference chemical environment. In other embodiments, the respective reference experimental conditions across which the baseline state is averaged vary slightly, such that the baseline state is representative of a number of similar conditions. For instance, in some embodiments where the baseline state will be compared to a perturbation state in which expression of a target gene is perturbed by siRNA, different instances of the reference experimental conditions may include cellular contexts that have been transformed with different control siRNA, e.g., that do not perturb expression of the target gene and/or do not perturb expression of any gene in the cellular context. In this fashion, background variance introduced by activation of the siRNA machinery within the cellular context, independent of perturbation of the target gene, can be accounted for through averaging of the baseline state. Similarly, in some embodiments, the chemical environment of different instances of the reference experimental conditions may be different, such that background variance introduced by shifts in the chemical environment, but independent of perturbation of a target gene or exposure to a test compound, can be accounted for through averaging of the baseline state.

As used herein, a “perturbation state” refers to a test experimental condition that includes an aliquot of a perturbed cellular context, which differs from a corresponding reference cellular context by a perturbation in the expression of a targeted gene, and a chemical environment that is the same as a corresponding reference chemical environment. That is, the perturbation state differs from a corresponding baseline state by altering the expression of a gene in the cellular context. Accordingly, the chemical environment of the perturbation state, aside from differenced caused by perturbation of the target gene, is the same as the chemical environment of a corresponding baseline state. In some embodiments, as described with reference to the baseline state, individual instances of the perturbation experimental conditions vary from each other, and are averaged together to represent the perturbation state. For instance, in some embodiments, different siRNA directed against the same target gene are used to perturb the expression of the target gene in different instances of the perturbation experimental conditions, e.g., to account for variance attributable to the off-target gene effects of a particular siRNA construct. Similarly, in some embodiments, the chemical environment of different instances of the perturbation experimental conditions may be different, e.g., to account for variance introduced by shifts in the chemical environment that are independent of perturbation of the target gene expression.

As used herein, a “compound state” refers to a test experimental condition that includes an aliquot of a cellular context that is the same as a corresponding reference cellular context and a chemical environment that differs from a corresponding reference chemical environment by the inclusion of a test compound. That is, the compound state differs from a corresponding baseline state by exposure of the cellular context to a test compound, e.g., a candidate non-biologic drug, a soluble factor, or a toxin. Accordingly, the cellular context of the compound state, aside from differences cause by exposure to the test compound, is the same as the cellular context of a corresponding baseline state. In some embodiments, as described with reference to the baseline state, individual instances of the compound experimental conditions vary from each other, and are averaged together to represent the compound state. For instance, in some embodiments, different control siRNA directed against the same target gene are transformed into aliquots of cellular contexts used in different instances of the compound experimental conditions. Similarly, in some embodiments, the chemical environment of different instances of the perturbation experimental conditions, aside from the test compound, may be different, e.g., to account for variance introduced by shifts in the chemical environment that are independent of the effects of exposure to the test compound.

As used herein, a “combination state” refers to a test experimental condition that includes an aliquot of a cellular context and a chemical environment, which differs from a corresponding reference experimental condition by perturbation of the expression of two target genes in the cellular context, perturbation of a target gene in the cellular context and exposure of the cellular context to a test compound, or exposure of the cellular context to two test compounds. Combination states can be used to determine whether the effects of two biological differences on a cellular context, e.g., perturbations of gene expression and/or exposure to test compounds, are synergistic, antagonistic, or independent of each other, thereby ascertaining whether the two biological differences interact with each other. In some embodiments, as described with reference to the baseline state, individual instances of the combination experimental conditions vary from each other, and are averaged together to represent the combination state. For instance, in some embodiments, different control siRNA directed against the same target gene are transformed into aliquots of cellular contexts used in different instances of the combination experimental conditions. Similarly, in some embodiments, the chemical environment of different instances of the perturbation experimental conditions, aside from test compounds, may be different, e.g., to account for variance introduced by shifts in the chemical environment that are independent of the effects of exposure to the test compound or perturbation of gene expression.

As used herein, a “cellular context” refers to a particular cell type. As used herein, perturbation of the expression of a target gene, relative to a reference cellular context, results in the creation of a cellular context that is different from the reference cellular context. Thus, an aliquot of cells representing a perturbation state are cells that are of the same cell type as the cells used in a corresponding baseline state, but in which the expression of a target gene has been perturbed. In some embodiments, individual instances of a particular cellular context (e.g., a reference cellular context or a test cellular context) vary from each other, and are averaged together to represent the particular cellular context. For instance, in some embodiments where the characteristics of a reference cellular context will be compared to the characteristics of a perturbed cellular context, in which expression of a target gene is perturbed by siRNA, different instances of the reference cellular context are be transformed with different control siRNA, e.g., that do not perturb expression of the target gene and/or do not perturb expression of any gene in the cellular context. In this fashion, background variance introduced by activation of the siRNA machinery within the cellular context, independent of perturbation of the target gene, can be accounted for through averaging of the characteristics from difference instances of the reference cellular context. Similarly, in some embodiments, different instances of a perturbed cellular context, in which expression of a target gene is perturbed by siRNA, different instances of the perturbed cellular context are transformed with different siRNA directed against the target gene, and are averaged are used to perturb the expression of the target gene in different instances of the perturbation experimental conditions, e.g., to account for variance attributable to the off-target gene effects of a particular siRNA construct.

As used herein, the terms “drug,” “candidate drug,” “small molecule candidate therapeutic agent,” and the like refer to a non-biological molecule that may be whose effect in a cell-based assay is of interest. In some embodiments, candidate drugs are part of a chemical screening library. Many commercial and proprietary chemical libraries exist, for example, the Diversity Compound Library (Charles River) contains 689,000 sourced compounds, the EXPRESS-Pick Collection Stock (Chem Bridge) contains over 480,000 chemical compounds, the CORE Library Stock contains more than 690,000 (Chem Bridge) compounds, and pharmaceutical companies have their own proprietary compound libraries having over a million compounds (Macarron R, et al., “Impact of high-throughput screening in biomedical research,” Nat Rev Drug Discov., 10(3):188-95 (2011), which is hereby incorporated by reference). However, the number of possible compounds is nearly limitless. For example, the PubChem database (see, Wang, Y., et al., Nucleic Acids Res. 40:D400-D412 (2012)), a public repository for screening data, lists over 93 million compounds for which screening data has been generated.

As used herein, the term “soluble factor” refers to a molecule secreted by a cell of a multicellular organism (e.g., a mammal, such as a human) into the extracellular space. In some embodiments, with reference to a cellular assay performed with a cell type from a particular multicellular organism, a soluble factor is a molecule that is secreted by a cell of that particular multicellular organism. For instance, in some embodiments, where a cellular assay is performed with human cells, a soluble factor is a molecule secreted by a human cell into the extracellular matrix. In other embodiments, a soluble factor is a protein secreted by a cell of a multicellular organism of the same class as an organism from which a cell used in a cellular assay was derived. For instance, in some embodiments, where a cellular assay used a mammalian cell, a soluble factor is a molecule secreted by a mammalian cell. Non-limiting examples of soluble factors include growth factors, chemokines, cytokines, adhesion molecules, proteases, and shed receptors. In some embodiments, a soluble factor is capable of regulating (e.g., activating, enhancing, deactivating, or down-regulating) a cellular pathway after being secreted into the extracellular space.

As used herein, the term “toxin” refers to a molecule produced by an organism other than an organism corresponding to a cell type used in a cellular assay, which has deleterious effects on the cell type used in the cellular assay.

As used herein, the term “compound” refers to any molecule whose effect in a cell-based assay is of interest. For example, in some embodiments, a compound refers to a small molecule candidate therapeutic agent, a biological molecule (e.g., a soluble factor, an antibody or portion thereof, or a candidate therapeutic nucleic acid), or a toxin.

As used herein, a “perturbation” of a cellular context is a change to the cellular context or surrounding environment that potentially results in a measureable change in at least one cellular phenotype. It will be appreciated that not all perturbations in fact cause a measurable change in cell context and the present disclosure is designed, at least in part, to ascertain whether perturbations do, in fact, cause such changes and, in some embodiments, to quantify such changes caused by them. In some embodiments, a perturbation is exposure of the cellular context to a compound that acts upon the cellular machinery of the cellular context, e.g., transfection of an siRNA that knocks-down expression of a gene in the cell or a chemical or biological compound that perturbs a cellular process (e.g., inhibits a cellular signaling pathway, inhibits a metabolic pathway, inhibits a cellular checkpoint, etc.). In some embodiments, a perturbation is a change to the cellular context itself, e.g., transduction of a CRISPR reagent that edits the genome of the cell

As used herein, a first perturbation and a second perturbation “interact” with each other when the perturbations affect a cell in a same or an opposite fashion, through a same or partially-redundant biological pathway. As such, some, but not all interactions, involve a physical interaction between the perturbation agents in vivo. For instance, a gene and a compound interact when the compound is a molecule that binds to and inhibits a function of the polypeptide encoded by the gene. However, a compound also interacts with a gene when, for example, the compound binds to and inhibits an activity of a downstream affector of the polypeptide encoded by the gene, even though the compound and the polypeptide encoded by the gene do not physically interact in vivo. Likewise, a first gene in a first biological pathway interacts with a second gene in a second pathway (or a compound that affects, e.g., inhibits or enhances) when the pathways have overlapping or partially-redundant functionality. For example, blood coagulation Factor VII and blood coagulation Factor IX both serve to activate blood coagulation Factor X to effect blood clotting. However, Factor VII functions through the Tissue Factor (extrinsic) coagulation pathway and Factor IX functions through the Contact activation (intrinsic) coagulation pathway).

### Methods and Systems for Compound Screening

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

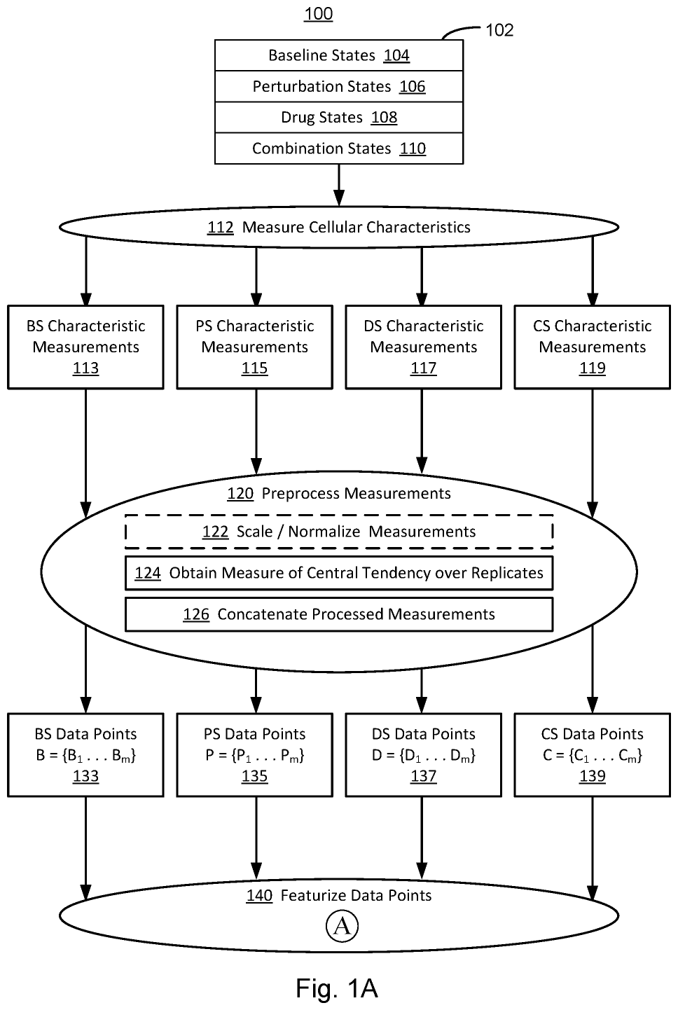

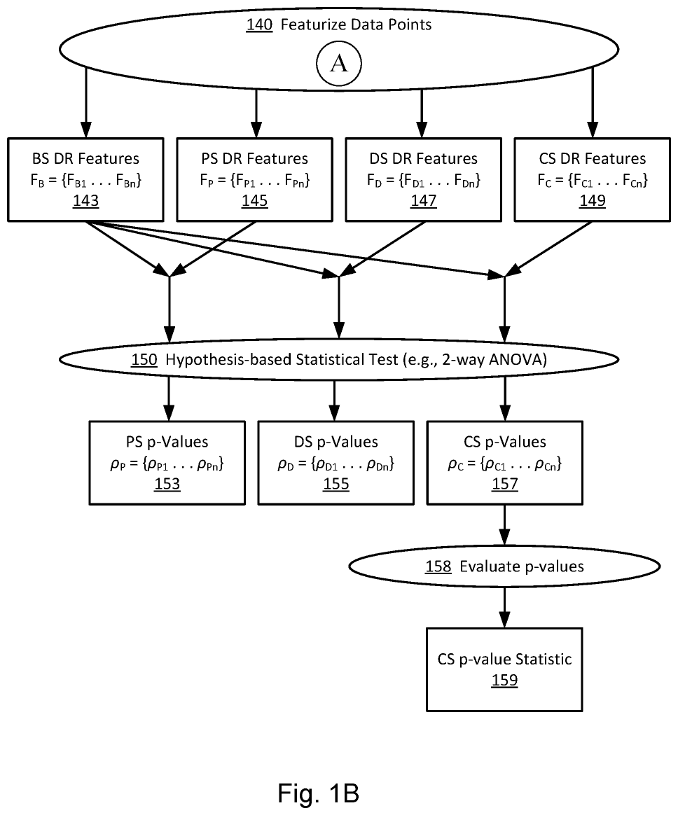

FIGS. 1A and 1B illustrate an example workflow 100, provided in some embodiments of the present disclosure, for identifying interactions within complex biological systems using a cell-based assay. FIGS. 1A and 1B makes reference to a specific embodiment for identifying an interaction between a gene and a candidate drug. However, it will be appreciated that by replacing one or both of the gene perturbation state(s) and candidate drug state(s) with a different state, e.g., a second candidate drug state(s), a second gene perturbation state(s), a soluble factor state(s), or a toxin state(s), that interactions between any of these types biological components can be identified using the same cell-based assay methodology as illustrated for gene-drug interactions in FIGS. 1A and 1B.

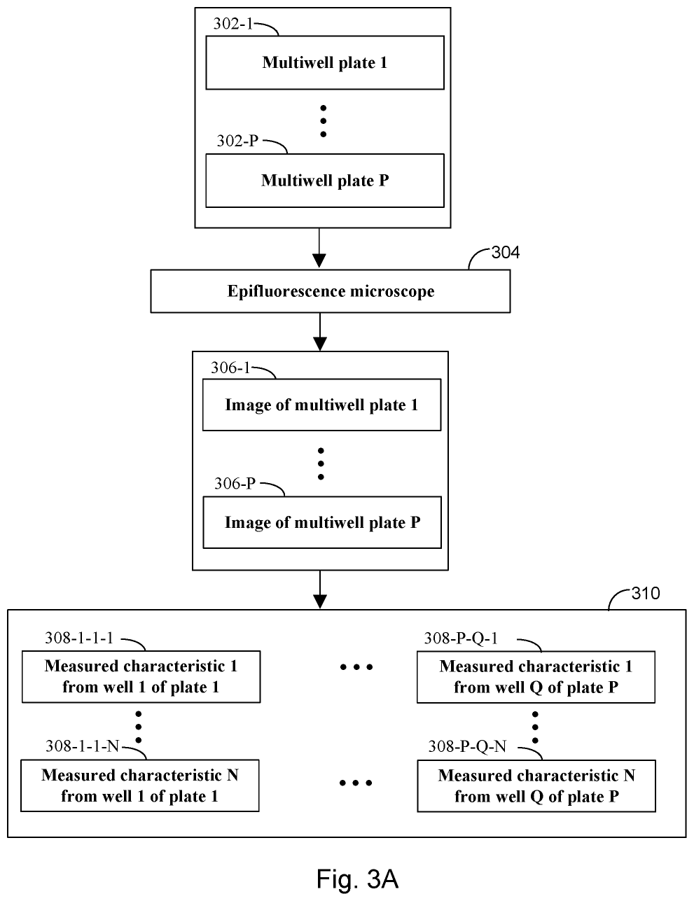

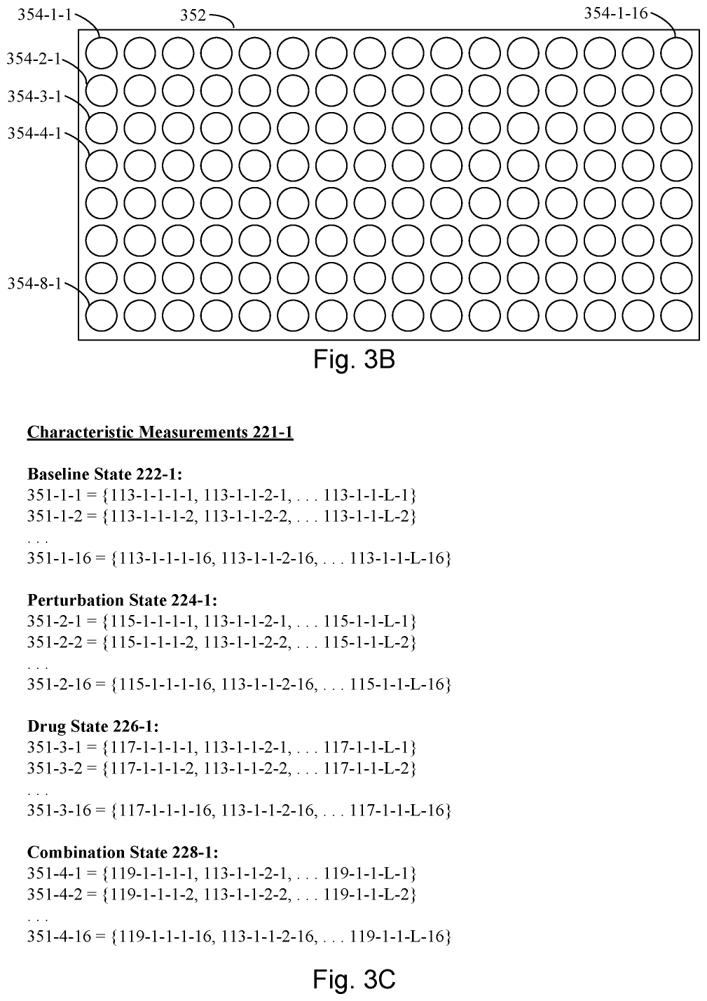

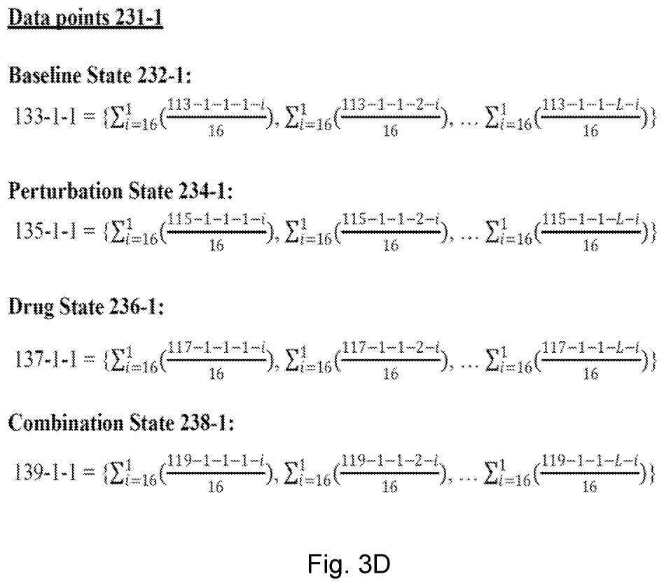

As illustrated in FIG. 1A, a baseline state 104, perturbation state 106, drug state 108, and combination state 110 are each represented by a plurality of experimental conditions established in the wells of one or more multiwell plates 102. For instance, referring to a hypothetical experiment illustrated in FIGS. 3B-3D, each well 354 in the first row of multiwell plate 352 (i.e., wells 354-1-1 through 354-1-16 in FIG. 3B) includes an experimental condition representative of baseline state 104, each well 354 in the second row (i.e., wells 354-2-1 through 354-2-16) includes an experimental condition representative of perturbation state 106, each well 354 in the third row (i.e., wells 354-3-1 through 354-3-16) includes an experimental condition representative of drug state 108, and each well 354 in the fourth row (i.e., wells 354-4-1 through 354-4-16) includes an experimental condition representative of combination state 110.

Each baseline state 104 includes an aliquot of cells representative of a baseline cellular context and a culture medium representative of a baseline chemical environment. For instance, referring to FIG. 3B, each of wells 354-1 in the first row of multiwell plate 352 includes an aliquot of cell type YFC (your favorite cells) in culture medium YFM (your favorite medium).

Each perturbation state 106 includes an aliquot of cells that correspond to the cells used in the baseline state, except that expression of a gene has been perturbed in the cells relative to expression of the gene in the cells representative of the baseline state. For instance, an siRNA or CRISPR reagent directed against the gene is introduced into an aliquot cells representative of the baseline state to perturb expression of the gene, thereby generating perturbed cells representative of the perturbation state. Each perturbation state also includes a culture medium representative of the baseline state, such that the only variable introduced into the perturbation state is the perturbed gene expression. For instance, referring to FIG. 3B, each of wells 354-2 in the second row of multiwell plate 352 includes an aliquot of cell type YFC into which an siRNA directed against gene YFG (your favorite gene) has been introduced, in culture medium YFM.

Each drug state 108 includes an aliquot of cells representative of a baseline cellular context and a culture medium representative of a baseline chemical environment. However, a candidate drug compound is added to the drug state, such that the only variable introduced into the drug state is the candidate drug compound. For instance, referring to FIG. 3B, each of wells 354-3 in the third row of multiwell plate 352 includes an aliquot of cell type YFC, culture medium YFM, and candidate drug YFD (your favorite drug).

Each combination state 110 includes an aliquot of cells that correspond to the cells used in the baseline state, except that expression of the gene perturbed in a corresponding perturbations state is also perturbed in the combination state, preferably in the same fashion as in the perturbation state. In addition, the combination state includes a culture medium representative of the baseline state, except that the candidate drug compound added a corresponding the drug state is also added to the combination state. In this fashion, two variables are introduced into the combination state, relative to the baseline state: the perturbation of gene expression and the presence of the candidate drug compound. For instance, referring to FIG. 3B, each of wells 354-4 in the fourth row of multiwell plate 352 includes an aliquot of cell type YFC into which an siRNA directed against gene YFG has been introduced, culture medium YFM, and candidate drug YFD.

After establishment of the baseline states 104, perturbation states 106, drug states 108, and combination states 110 in the multiwell plate(s), the cells are incubated for a period of time sufficient to allow for changes in cellular phenotypes. The period of time for which the cells are incubated in the multiwell plate will depend upon factors known to the skilled artisan, such as the cell types, the culture medium used, the expected effects of one or more perturbations and/or candidate drug compounds, the growth status of the cells, etc. After incubation, the cells are optionally fixed and/or stained, to facilitate measurement of cellular characteristics. In some embodiments, cells in the various states are painted, to facilitate measurement of various cell morphologic characteristics. Methods of cell painting are well known in the art. See, for example, Bray Mass., et al., Nat. Protoc., 11(9):1757-74 (2016), the content of which is incorporated herein by reference.

Next, characteristics of the cells in each instance of the baseline states 104, perturbation states 106, drug states 108, and combination states 110, are measured (112). In some embodiments, the cellular characteristics are measured using optical imaging, e.g., as described in Bray Mass. et al., supra, with respect to cell painting. Other methods for cell imaging and measurement of optical characteristics, as well as methods for measurement of non-optical characteristics, useful in conjunction with the workflows provided herein are described further below. The sets of baseline state characteristic measurements 113, perturbation state characteristic measurements 115, drug state characteristic measurements 117, and combination state characteristic measurements 199 are representative of each respective state. For instance, referring to the hypothetical experimental set-up above, with reference to FIGS. 3B-3D, L cellular characteristics are measured in each of wells 354-1-1 through 354-4-16, such that 16 sets of L characteristics are measured for each experimental state, as shown in FIG. 3C.

The raw measurement sets are then pre-processed (120), to form a baseline state data point 133, perturbation state data point 135, drug state data point 137, and combination state data point 139. In some embodiments, the data is scaled or normalized (122) across the raw data set. Methods for data scaling and data normalization are known in the art, e.g., as described further herein below. A measure of central tendency for each measured characteristic is then obtained (124) from the raw or scaled and/or normalized data across each replicate for each experimental state. The measures of central tendency are then concatenated (126) into data points for each of the experimental states. Each data point is a multidimensional vector containing the measure of central tendency of each characteristic measurement acquired across a plurality of instances of the respective experimental state. For example, referring to the hypothetical experiment set-up described above and illustrated in FIGS. 3B-3D, where the measure of central tendency is a mean, each data point (e.g., baseline state data point 133, perturbation state data point 135, drug state data point 137, and combination state data point 139, as illustrated in FIG. 3D) is a set of the measurement of each of the L characteristics averaged across the 16 experimental instances representative of the respective experimental state.

Next, the data points for each experimental state are featurized (140), to reduce the dimensionality of the data, thereby enhancing sparse datasets. In addition to enriching meaningful data in the data sets, featurization reduces the amount of data that needs to be processed by the system, reducing the time needed to perform downstream analysis, thereby improving the performance of the computer. Examples of methods that reduce a data set, while maintaining information that explains the variability in the data set, include principal component analysis (PCA), and application of neural networks. For example, in some embodiments, data points 133, 135, 137, and 139 are applied to a set of principal components, previously trained against training states (e.g., training baseline states, training perturbation states, training drug states, and/or training combination states) to generate sets of principal component values. In other embodiments, data points 133, 135, 137, and 139 are applied to an artificial neural network, previously trained against training states (e.g., training baseline states, training perturbation states, training drug states, and/or training combination states), and a hidden layer of the neural network (e.g., an embedding layer) having fewer dimensions than the data points is acquired for further analysis. The result of the featurization of the data points are dimension reduced (DR) feature sets, e.g., as shown in FIG. 1B, baseline state DR feature set 143, perturbation state DR feature set 145, drug state DR feature set 147, and combination state DR feature set 149.

Next, a hypothesis-based statistical test is applied to the dimension reduced feature sets (150), to determine whether there is a statistically significant interaction between the effects of the gene expression perturbation and the effects of the candidate drug exposure on one or more cellular characteristics, suggesting that the gene and drug operate through a same or partially redundant pathway in vivo. That is, suggesting that the drug interacts with the product of the gene in vivo. That is, if disruption of gene expression and exposure to the drug affect the same biological pathway in the same fashion, in vivo, it could be expected that the combination of disrupting the gene's expression in the cells and exposing the cells to the compound, would have less than an additive effect on changes in the cellular characteristics. Similarly, if disruption of gene expression and exposure to the drug affect partially redundant biological pathways in the same fashion, in vivo, it could be expected that the combination of disrupting the gene's expression in the cells and exposing the cells to the compound, would have more than an additive effect on changes in the cellular characteristics, e.g., a synergistic effect.

In some embodiments, as illustrated in FIG. 1B, the hypothesis-based statistical test is a 2-way ANOVA, that determines p-values 153 for the significance of the gene expression perturbation's effects on changes to each of the features in the featurized data sets, p-values 155 for the significance of the candidate drug's effects on changes to each of the features in the featurized data sets, and p-values for the significance of the interaction between the gene expression perturbation and candidate drug effects on changes to each of the features in the featurized data sets. The resulting p-values 157 for the interaction between the gene perturbation and candidate drug exposure is then evaluated (158) to determine whether the interaction between the two variables has a statistically significant effect on the features. In some embodiments, as illustrated in FIG. 1B, the p-values are combined to generate a p-value statistic 159. Methods for evaluating sets of p-values, such as Fischer's method, as described herein further below.

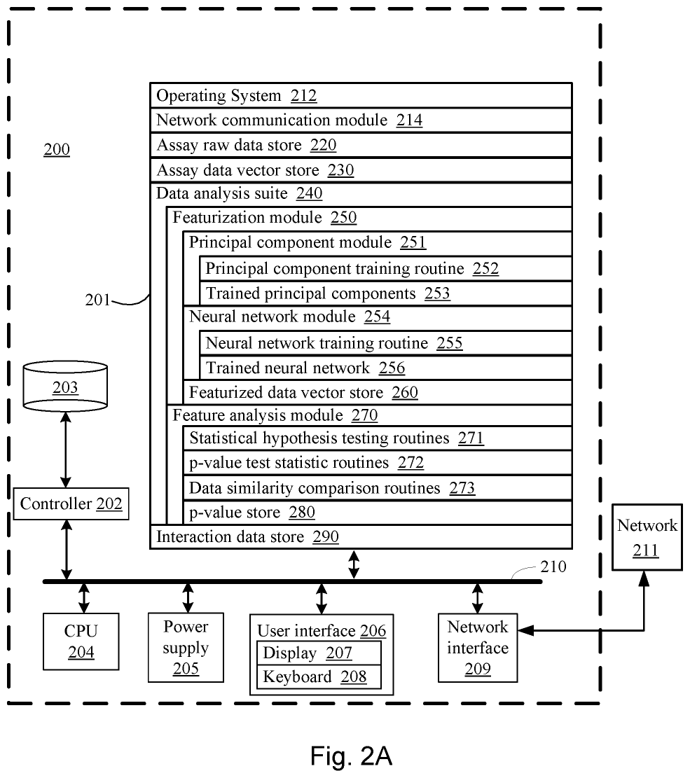

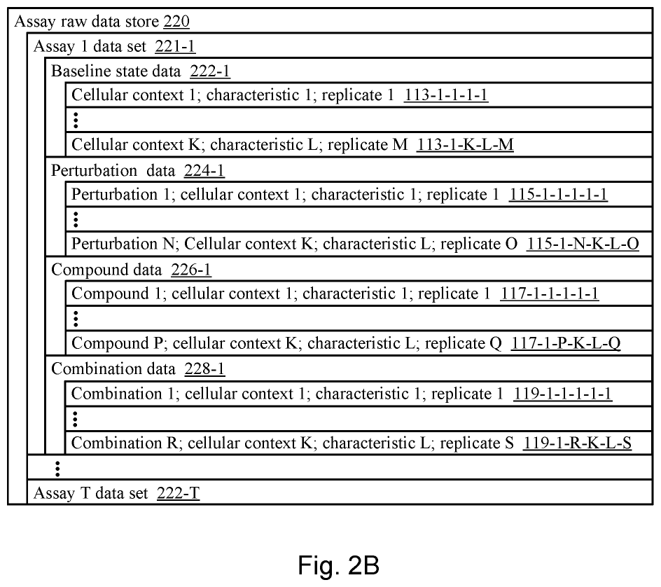

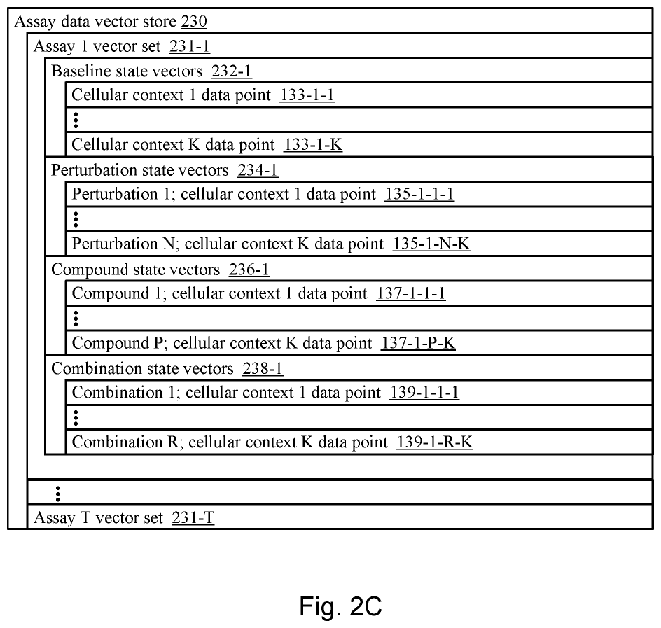

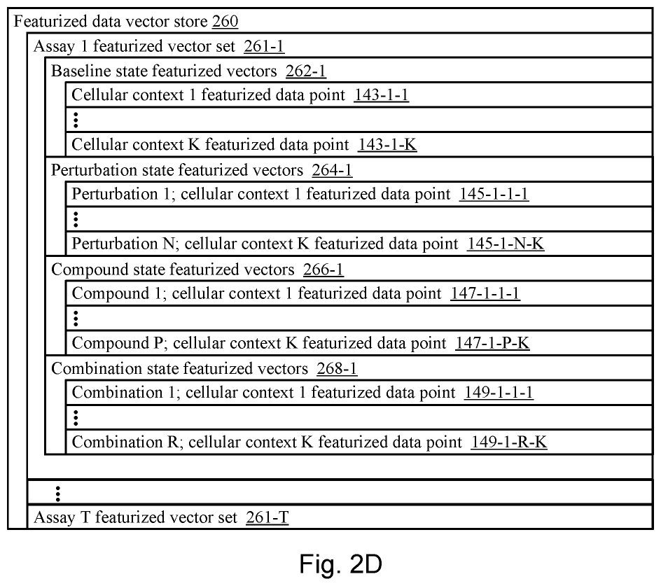

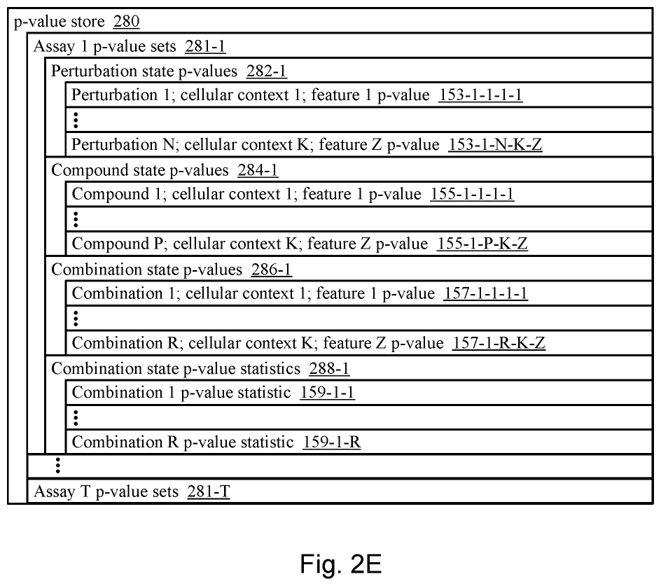

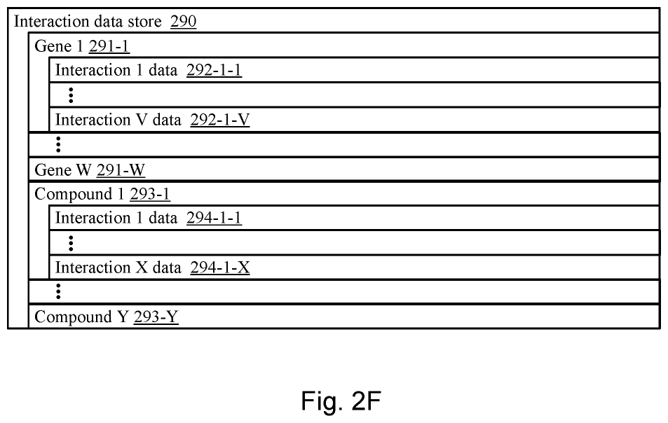

A detailed description of a system 200 for identifying interactions within complex biological systems using data from a cell-based assay is described in conjunction with FIGS. 2A-2F. As such, FIGS. 2A-2F collectively illustrate the topology of a system, in accordance with an embodiment of the present disclosure.

Referring to FIG. 2A, in typical embodiments, system 200 comprises one or more computers. For purposes of illustration in FIG. 2A, system 200 is represented as a single computer that includes all of the functionality for identifying interactions within complex biological systems using data from a cell-based assay. However, the disclosure is not so limited. In some embodiments, the functionality for identifying interactions within complex biological systems using data from a cell-based assay is spread across any number of networked computers and/or resides on each of several networked computers and/or is hosted on one or more virtual machines at a remote location accessible across the communications network 211. One of skill in the art will appreciate that any of a wide array of different computer topologies are used for the application and all such topologies are within the scope of the present disclosure.

With the foregoing in mind, an example system 200 for identifying interactions within complex biological systems using data from a cell-based assay includes one or more processing units (CPU's) 204, a network or other communications interface 209, a memory 201 (e.g., random access memory), one or more magnetic disk storage and/or persistent devices 203 optionally accessed by one or more controllers 202, one or more communication busses 210 for interconnecting the aforementioned components, a user interface 206, the user interface 206 including a display 207 and input 208 (e.g., keyboard, keypad, touch screen), and a power supply 205 for powering the aforementioned components. In some embodiments, data in memory 201 is seamlessly shared with non-volatile memory 203 using known computing techniques such as caching. In some embodiments, memory 201 and/or memory 203 includes mass storage that is remotely located with respect to the central processing unit(s) 204. In other words, some data stored in memory 201 and/or memory 203 may in fact be hosted on computers that are external to the system 200 but that can be electronically accessed by the system 200 over an Internet, intranet, or other form of network or electronic cable (illustrated as element 211 in FIG. 2) using network interface 209.

In some embodiments, the memory 201 of the system 200 for identifying interactions within complex biological systems using data from a cell-based assay include:
an operating system 212 that includes procedures for handling various basic system services;
a network communications module 214 for connecting the system 200 with other devices and/or a communication network 211;
an assay raw data store 220 for storing measurements of cellular characteristics acquired from experimental conditions representative of an experimental state (e.g., data sets 221, as illustrated in FIG. 2B, containing one or more of measurements 113 from baseline state experimental conditions 222, measurements 115 from perturbation state experimental conditions 224, measurements 117 from compound state experimental conditions 226, and/or measurements 119 from combination state experimental conditions 228);
an assay data store 230 for storing data points generated from various experimental states from assay raw data 220 (e.g., vector sets 231, as illustrated in FIG. 2C, containing one or more of data points 133 from baseline experimental states 232, data points 135 from perturbation experimental states 234, data points 137 from compound experimental states 236, and/or data points 139 from combination experimental states 238);
a data analysis suite 240 including instructions for analyzing data points generated from experimental states, the data analysis suite including:
   a featurization module 250 for reducing the dimensions of data points, the featurization module optionally including one or both of a principal component module 251 and a neural network module 253, the featurization module also including a featurized data vector store 260 for storing feature sets generated from data vector sets 231 (e.g., featurized vector sets 261, as illustrated in FIG. 2D, containing one or more of featurized data points 143 from baseline experimental states 262, featurized data points 145 from perturbation experimental states 264, featurized data points 147 from compound experimental states 266, and/or featurized data points 149 from combination experimental states 268), where:
      principal component module 251 applies data vector sets 231 to trained principal components 253 to generate principal component values, stored as featurized vector sets 261,
      principal component module 251 optionally contains training routine 252, for learning principal components 253 from training data sets of characterization measurements (e.g., measures of central tendency of measurements across a plurality of instances of various baseline, perturbation, compound, and/or combination training states),
      neural network module 254 applies data vector sets 231 to trained neural networks, or relevant portions thereof, to obtain values from a hidden layer (e.g., an embedding layer) of the neural network, stored as featurized vector sets 261,
      neural network module 254 optionally contains training routine 255, for training neural networks 256 from training data sets of characterization measurements (e.g., measures of central tendency of measurements across a plurality of instances of various baseline, perturbation, compound, and/or combination training states);
a feature analysis module 270 for analyzing featurized vector sets 261 to determine whether two biological agents (e.g., two of a gene, a candidate drug compound, a soluble factor, and a toxin) tested within the cell-based assay interact with each other, feature analysis module 270 including:
   a statistical hypothesis testing routine 271 for determining the significance of a perturbed experimental state (e.g., a perturbation state 106, a drug state 108, and/or a combination state 110) on one or more measured cellular characteristics, which generates p-value sets 281 for an assay,
   an optional p-value statistic routine (272), for combining p-values for an experimental state (e.g., perturbation state p-values 153, compound state p-values 155, or combination state p-values 157) to generate p-value statistics for an experimental state (e.g., combination p-value statistic 159),
   a data similarity comparison routine 273 for comparing pairs of featurized vector sets 261 (e.g., comparing one of a perturbation state featurized data point 145 or a compound state featurized data point 147 to one of a different perturbation state featurized data point 145 or compound state featurized data point 147), for identifying different biological features that similarly effect one or more cellular characteristics, and
   a p-value store 280 for storing p-value sets 281 generated for features of various experimental states from featurized vector sets 261 (e.g., p-value sets 281, as illustrated in FIG. 2E, containing one or more of p-values 153 for peturbation experimental states 282, p-values 155 for compound experimental states 284, p-values 157 for combination experimental states 286, and/or p-values 159 for combination experimental states 288); and
an interaction data store 290 for storing the results of various interaction tests for gene perturbations 291 (e.g., data 292 about gene interactions with various biological features, e.g., other genes, candidate drugs, soluble factors, and/or toxins) and compound perturbations 293 (e.g., data 294 about compound interactions with various biological features, e.g., other genes, candidate drugs, soluble factors, and/or toxins).

In some embodiments, modules 214, 250, 251, 254, and/or 270, and or data stores 220, 230, 260, 280, and/or 290 are accessible within any browser (e.g., installed on a phone, tablet, or laptop/desktop system). In some embodiments, modules 214, 250, 251, 254, and/or 270 run on native device frameworks, and are available for download onto the system 200 running an operating system 212, such as Android or iOS.

In some implementations, one or more of the above identified data elements or modules of the system 200 for identifying interactions within complex biological systems using data from a cell-based assay are stored in one or more of the previously described memory devices, and correspond to a set of instructions for performing a function described above. The above-identified data, modules or programs (e.g., sets of instructions) need not be implemented as separate software programs, procedures or modules, and thus various subsets of these modules may be combined or otherwise re-arranged in various implementations. In some implementations, the memory 201 and/or 203 optionally stores a subset of the modules and data structures identified above. Furthermore, in some embodiments the memory 201 and/or 203 stores additional modules and data structures not described above.

In some embodiments, device 200 for identifying interactions within complex biological systems using data from a cell-based assay is a smart phone (e.g., an iPHONE), laptop, tablet computer, desktop computer, or other form of electronic device. In some embodiments, the device 200 is not mobile. In some embodiments, the device 200 is mobile.

Referring to FIG. 3A, in some embodiments, the present disclosure relies upon the acquisition of a data set 221 that includes measurements of a plurality of cellular characteristics 308 (e.g., baseline state measurements 113, perturbation state measurements 115, compound state measurements 117, and/or combination state measurements 119) for various experimental states, in one or more replicates, and in one or more cell contexts. As an example, each experimental state i in a plurality of M experimental states is introduced into wells of a multiwell plate 302 for each of/cell contexts in j instances, resulting in X wells containing experimental state i, where X=(j)*(l). N cellular characteristics are then measured from each well {1 . . . Q} of each multiwell plate {1 . . . P}, resulting in N*M*X*cellular characteristic measurements for the experimental states.

In some embodiments, referring to FIG. 3A, these cellular characteristic measurements are acquired by capturing images 306 (e.g., 306-1 to 306-P) of the multiwell plates using, for example, epifluorescence microscopy 304. The images 306 are then used as a basis for obtaining the measurements of the N different characteristics from each of the wells in the multiwell plates, thereby forming dataset 310 (e.g., data set 221 illustrated in FIGS. 2B and 3C). Data set 310 is used to generate data set 231, which include multidimensional data points containing measures of central tendency of cellular characteristic measurements across a plurality of instances for each experimental state (e.g., one or more data points for a baseline state 133, perturbation state 135, compound state 137, and/or combination state 139, as illustrated in FIGS. 2C and 3D). These data points are then used to generate featurized vector set 261 (e.g., including baseline state featurized data points 143, perturbation state featurized data points 145, compound state featurized data points 147, and/or combination state featurized data points 149, as illustrated in FIG. 2D) which, in turn, are used to evaluate interactions between biological agents (e.g., genes, candidate drug compounds, soluble factors, and/or toxins), e.g., as described above with reference to FIG. 1, or evaluate the similarity between the effects of pairs of biological agents.

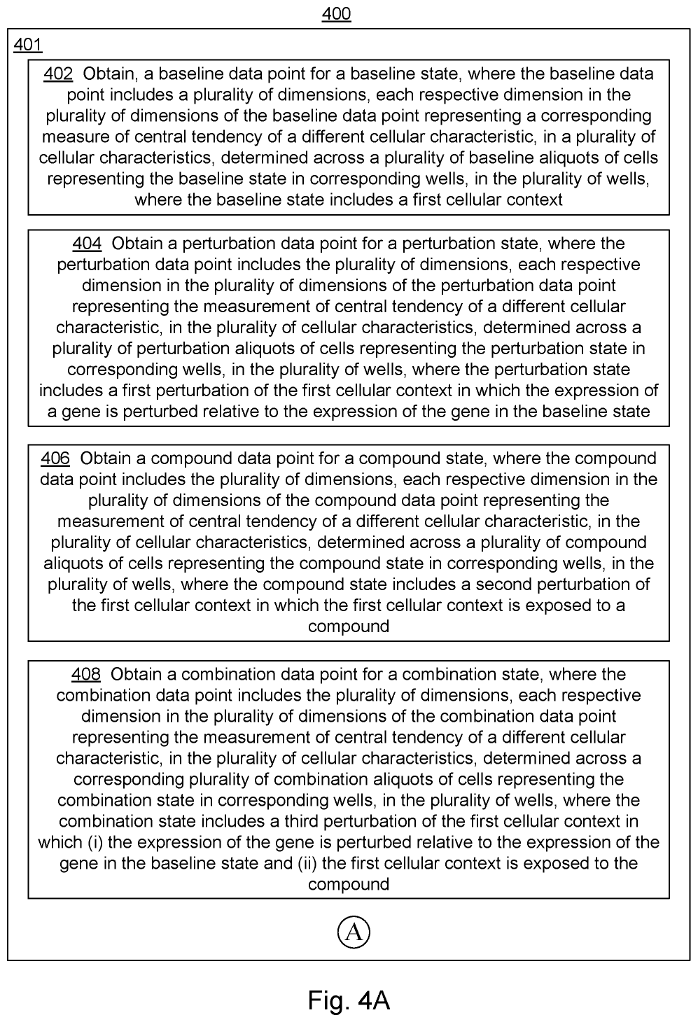

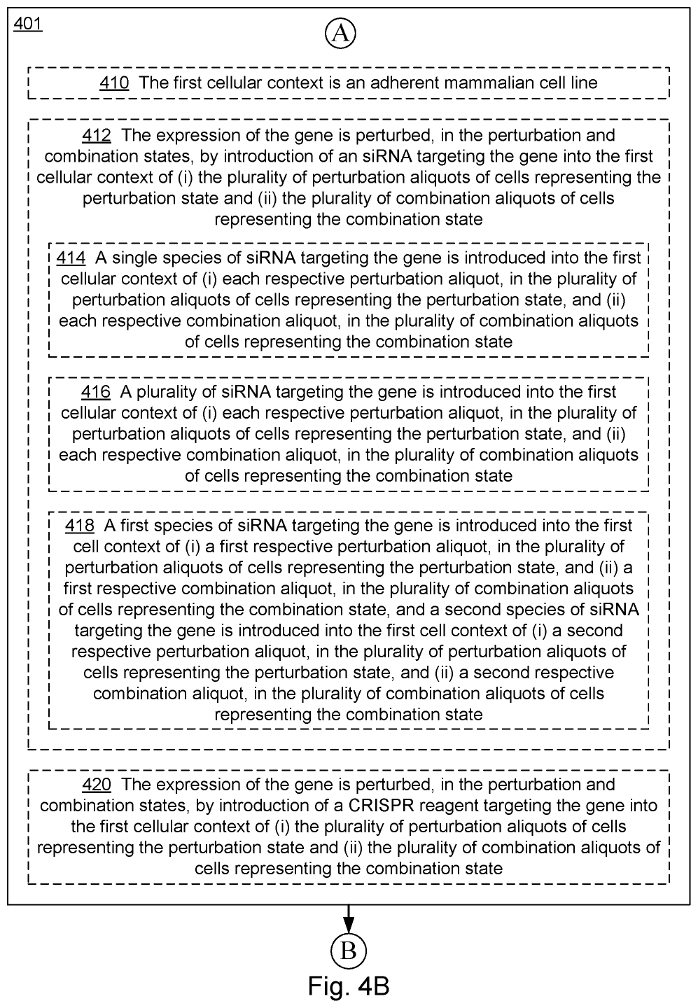

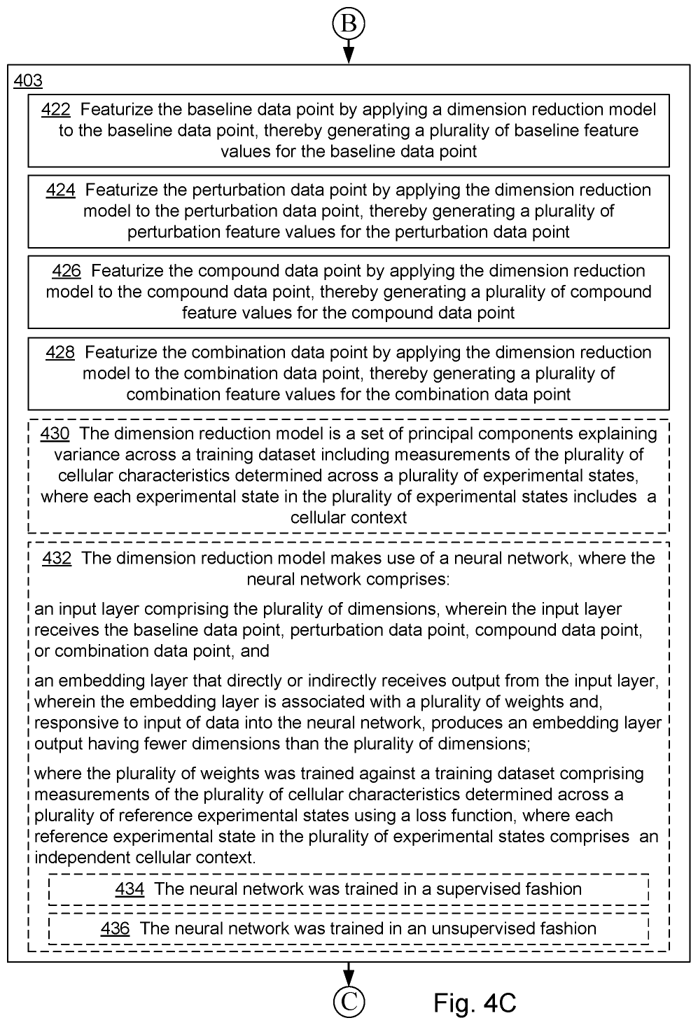

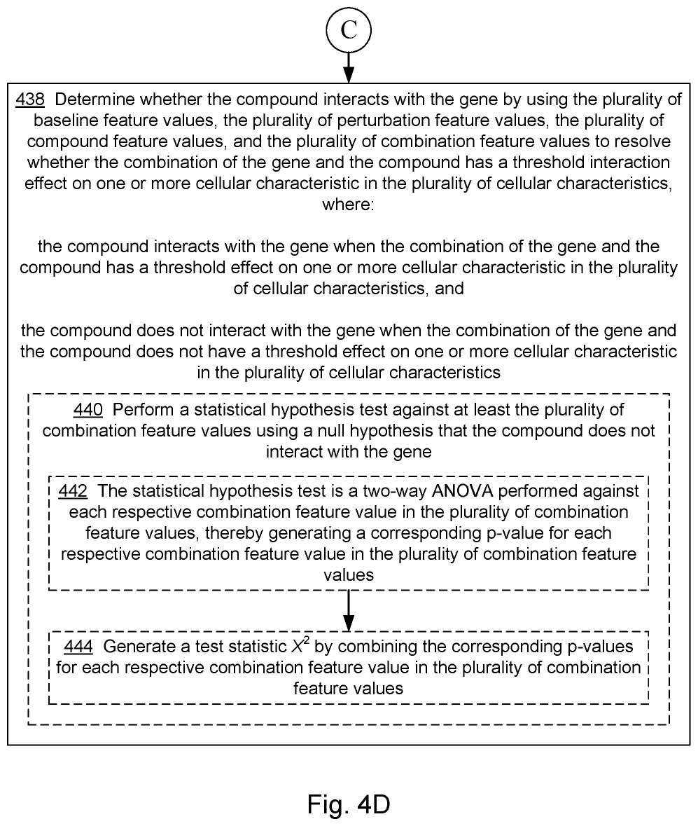

Now that details of a system 200 for identifying interactions within complex biological systems using data from a cell-based assay have been disclosed, details regarding a processes and features of the system, in accordance with an embodiment of the present disclosure, are disclosed below. Example processes are also described with reference to FIGS. 4A-4D, 5A-5H, 6A-6D, 7A-7F, and 8A-8D. In some embodiments, such processes and features of the system are carried out by modules 214, 250, 251, 254, and/or 270, as illustrated in FIG. 2. Referring to these methods, the systems described herein (e.g., system 200) include instructions for performing the methods for evaluating an effect of one or more perturbations and/or therapeutic candidate compounds on a cell.

In one aspect, the disclosure provides a method 400 for determining whether a compound interacts with a gene, in a cell based assay. In some embodiments, the compound is a putative drug candidate, for example, a candidate therapeutic compound from a chemical library. In some embodiments, the compound is a soluble factor, e.g., a growth factor, chemokine, cytokine, adhesion molecule, protease, or shed receptor. In some embodiments, the compound is a toxin. The cell based assay is performed in a plurality of wells across one or more multiwell plates. For example, referring to the hypothetical example described above with reference to FIGS. 3A-3D, different instances of experimental states (e.g., baseline state 104, perturbation state 106, compound state 108, and combination state 110) are established in different wells 354 (e.g., in well rows 354-1 to 354-8) of multiwell plate 352.

In some embodiments, as illustrated with reference to FIG. 1A, the methods described herein include establishing a plurality of instances of cell-based experimental conditions representative of experimental states (e.g., baseline states 104, perturbation states 106, compound states 108, and/or combination states 110), e.g., in the wells of one or more multiwell plate, and measuring cellular characteristics from each of the instances, thereby generating a raw data set 221 for the assay (e.g., containing characteristic measurements 113, 115, 117, and/or 119 for one or more corresponding baseline experimental states 222, perturbation experimental states 224, compound experimental states 226, and/or combination experimental states 228). In some embodiments, the measurements and/or combinations of measurements of cellular characteristics are generated from one or more images of wells corresponding to an experimental state (e.g., wells corresponding to a baseline state 104, a perturbation state 106, a compound state 108, and/or a combination state 110), e.g., using an image analysis package, such as CellProfiler™ (Ljosa and Carpenter, PLoS Comput Biol., 5(12):e1000603 (2009) which is hereby incorporated by reference herein). In some embodiments, each feature is derived from a combination of measurable characteristics selected from a color, a texture, and a size of the cell context, or an enumerated portion of the cell context.

The raw data (e.g., the characteristic measurements of each instance of an experimental state) contained in data set 221 is then used to generate a set of data points 231 for each corresponding experimental state (e.g., data points 133 for a baseline experimental state 232, 135 for a perturbation experimental state 234, 137 for an experimental compound state 236, and/or 139 for an experimental combination state 238). In other embodiments, the methods described herein begin with the processing of raw data sets 221 or data point sets 231. That is, in some embodiments, data obtained from cell-based assays, performed as described herein, is received by system 200, and the methods described herein use that data to identify interactions between various biological agents, e.g., with respect to method 400, interactions between a gene and a compound.

Method 400 begins with a block 401 which is illustrated in FIGS. 4A and 4B. Method 400 includes obtaining (402) a baseline data point for a baseline state (e.g., baseline data point 133, as illustrated in FIGS. 1A, 2C, and 3D, obtained from measurements of cellular characteristics in instances of experimental conditions representative of a baseline state 104). The baseline data point includes a plurality of dimensions, where each respective dimension in the plurality of dimensions of the baseline data point represents a corresponding measure of central tendency of a different cellular characteristic, e.g., in a plurality of cellular characteristics, determined across a plurality of baseline aliquots of cells representing the baseline state, where the baseline state includes a first cellular context.

For instance, each baseline cellular characteristic 113 is measured in each instance of an experimental condition representative of baseline state 104 in the assay (e.g., measurements 113-1-1 through 113-1-16 of the same characteristic are obtained from wells 354-1-1 through 354-1-16, respectively, in FIG. 3B) and then a measure of central tendency is determined across each of the measurements (e.g., the mean of measurements 113-1-1 through 113-1-16 of the first characteristic

$$( {\sum }_{i = 16}^{1} ( \frac{113 - 1 - 1 - 1 - i}{16} ) ) ,$$

as illustrated in FIG. 3D). The measures of central tendency for each of the characteristics representative of the experimental state are then concatenated into a baseline data point (e.g., data point 133) for the respective cellular context. In some embodiments, the measure of central tendency is an arithmetic mean, weighted mean, midrange, midhinge, trimean, geometric mean, geometric median, Winsorized mean, median, or mode of the cellular characteristic.

In some embodiments, each of the cellular characteristics is an optically-measureable characteristic. In other embodiments, at least one cellular characteristic in the plurality of cellular characteristics is measured using a non-optical measurement. Non-limiting examples of optically-measureable and non-optically measureable cellular characteristic are described herein, e.g., in the Cellular Characteristic sections provided below.

In some embodiments, each of the baseline aliquots of cells include a single cell type, e.g., a single type of mammalian cell. In some embodiments, each of the baseline aliquots of cells includes the same mixture of different cell types, e.g., a co-culture of two different mammalian cells. In yet other embodiments, the plurality of baseline aliquots of cells includes different cellular contexts in different instances of the experimental condition that is representative of the baseline state. As described in further detail below, in some embodiments, measurements of the same cellular characteristic are acquired from different cell types in order to account for changes in the characteristic, e.g., upon perturbation of gene expression, exposure to a compound, and/or both, that are specific to one cell type. For instance, referring back to the hypothetical experiment described above with reference to FIGS. 3B-3D, in some embodiments, each baseline experimental condition in wells 354-1-1 to 354-1-16 contains a different cell type, or every two wells across the first row contain a different cell type, or every three wells across the first row contain a different cell type, etc.

In one embodiment, the first cellular context is a mammalian cell line. In one embodiment, the first cellular context is an adherent mammalian cell line (410). In some embodiments, the first cellular context is a human cell. In some embodiments, the first cellular context is a primary human cell. Non-limiting examples of cell types useful for the methods provided herein are described herein, e.g., in the Cell Contexts section provided below.

Method 400 also includes obtaining (404) a perturbation data point for a perturbation state (e.g., perturbation data point 135, as illustrated in FIGS. 1A, 2C, and 3D, obtained from measurements of cellular characteristics in instances of experimental conditions representative of a perturbation state 106). The perturbation data point includes the plurality of dimensions (e.g., the same number of dimensions as the corresponding baseline data point 133), each respective dimension in the plurality of dimensions of the perturbation data point representing the measurement of central tendency of a different cellular characteristic, e.g., in the plurality of cellular characteristics (the same cellular characteristics that were measured for the baseline state), determined across a plurality of perturbation aliquots of cells representing the perturbation state (e.g., referring to the hypothetical example with reference to FIG. 3B, the cellular characteristics are measured for each well 354-2 across the second row of multiwell plate 352, each of which contains an instance of an experimental condition representative of the perturbation state).

The perturbation state includes a first perturbation of the first cellular context in which the expression of a gene is perturbed relative to the expression of the gene in the baseline state. That is, the background for the cellular context(s) used in the perturbation experimental conditions is the same as the cellular context used in the background experimental conditions. However, the expression of a target gene in the background cellular context is perturbed relative to the expression of the target gene in the baseline cellular contexts. As described with reference to the baseline state above, in some embodiments, the same cellular context is used in each of the perturbation experimental conditions (e.g., when the same cellular context is used in each of the baseline experimental conditions). In other embodiments, different cellular contexts are used in different instances of the perturbation experimental conditions (e.g., when different cellular contexts are used in different instances of the baseline experimental conditions). The point is that it is advantageous to use as close to the same cellular background, as possible, in the experimental conditions corresponding to the baseline state and the experimental conditions corresponding to the perturbation state, so that differences in the cellular characteristics of the perturbation state, relative to the baseline state, can be confidently attributable to the perturbation of the target gene.

As outlined above, because the methods provided herein are not tied to any particular cellular dysfunction or disease model, the expression of any gene in the cellular context may be perturbed, to identify interactions with that gene and a second biological agent (e.g., another gene, a candidate drug compound, a soluble factor, or a toxin). Moreover, unlike conventional synthetic lethality assays used to identify interactions between biological agents, interactions between agents that do not cause drastic changes in a cellular feature (e.g., like apoptosis) can still be identified, broadening the utility of the methods described herein beyond conventional methodologies for identifying interactions in a complex biological system.

In some embodiments, the expression of the target gene is perturbed, in the perturbation state, by introduction of an siRNA targeting the gene into the first cellular context of the plurality of perturbation aliquots of cells representing the perturbation state (412). For instance, the cells included in wells 354-2-1 through 354-2-16, representing the perturbation state in the hypothetical example illustrated in FIG. 3B, are the same cells included in wells 354-1-1 through 354-1-16, representing the baseline state, except that one or more siRNA directed to the target gene has been introduced into the cells included in wells 354-2-1 through 354-2-16, but not into the cells included in wells 354-1-1 through 354-1-16.

In some embodiments, a single species of siRNA targeting the gene (e.g., siRNA with a single, defined sequence) is introduced into the first cellular context of each respective perturbation aliquot, in the plurality of perturbation aliquots of cells representing the perturbation state (414). That is, in some embodiments, for every gene that interaction data is being queried, a single siRNA sequence is used in each instance of the perturbation state. In some embodiments, a plurality of siRNA targeting the gene is introduced into the first cellular context of each respective perturbation aliquot, in the plurality of perturbation aliquots of cells representing the perturbation state (416). That is, in some embodiments, multiple siRNA sequences are used to perturb the expression of the target gene.

In yet other embodiments, a first species of siRNA targeting the gene is introduced into the first cell context of a first respective perturbation aliquot, in the plurality of perturbation aliquots of cells representing the perturbation state, and a second species of siRNA targeting the gene is introduced into the first cell context of a second respective perturbation aliquot, in the plurality of perturbation aliquots of cells representing the perturbation state (418). That is, in some embodiments, different siRNA molecules that target a different portion (sequence) of the target gene are used in different instances of the perturbation state. For instance, referring to the hypothetical example illustrated in FIG. 3B, a first siRNA directed to a targeted gene is introduced into cells used in well 354-2-1 of plate 352, and a second siRNA directed to a different sequence in the targeted gene is introduced into cells used in well 354-2-2 (or every other well, every third well, etc,), such that the characteristics represented in the resulting perturbation data point 115 are measures of central tendencies of the characteristic measured across cells in which the targeted gene is perturbed using difference siRNA species. It is known that some siRNA perturb the expression of genes other than the target gene. These off-target affects, depending on the extent to which they affect the expression of the off-target gene, may significantly affect the cellular characteristics measured in the assays described herein. By using a plurality of different siRNA sequences directed to the target gene, changes in the cellular characteristics from any particular off-target effect can be averaged out, since different off-target genes would be expected to be affected by different siRNA sequences.

In some embodiments, the expression of the gene is perturbed, in the perturbation state, by introduction of a CRISPR reagent targeting the gene into the first cellular context of the plurality of perturbation aliquots of cells representing the perturbation state (420). For instance, the cells included in wells 354-2-1 through 354-2-16, representing the perturbation state in the hypothetical example illustrated in FIG. 3B, are the same cells included in wells 354-1-1 through 354-1-16, representing the baseline state, except that the target gene has been altered by one or more CRISPR reagents in the cells included in wells 354-2-1 through 354-2-16, but not into the cells included in wells 354-1-1 through 354-1-16. More details with respect to methods for perturbing gene expression are described herein, e.g., in the Gene Expression Perturbation section provided below.

Method 400 also includes obtaining (406) a compound data point for a compound state (e.g., compound data point 137, as illustrated in FIGS. 1A, 2C, and 3D, obtained from measurements of cellular characteristics in instances of experimental conditions representative of a compound state 108). The compound data point includes the plurality of dimensions (e.g., the same number of dimensions as the corresponding baseline data point 133 and perturbation data point 135), each respective dimension in the plurality of dimensions of the compound data point representing the measurement of central tendency of a different cellular characteristic, in the plurality of cellular characteristics (the same cellular characteristics that were measured for the corresponding baseline state and perturbation state), determined across a plurality of compound aliquots of cells representing the compound state (e.g., referring to the hypothetical example with reference to FIG. 3B, the cellular characteristics are measured for each well 354-3 across the third row of multiwell plate 352, each of which contains an instance of an experimental condition representative of the compound state).

The compound state includes a second perturbation of the first cellular context in which the first cellular context is exposed to a compound. That is, the cellular context(s) used in the compound experimental conditions is the same as the cellular context used in the background experimental conditions, as well as the basis for the cellular context used in the corresponding perturbation experimental conditions. However, the cellular context is exposed to a test compound, e.g., a candidate drug, a soluble factor, or a toxin. In some embodiments, the compound is a candidate drug compound, such that the method is for identifying an interaction between a gene and a candidate drug compound. In some embodiments, the compound is a soluble factor, such that the method is for identifying an interaction between a gene and a soluble factor. In some embodiments, the compound is a toxin, such that the method is for identifying an interaction between a gene and a toxin. More details with respect to compounds useful for method 400 are described herein, e.g., in Compound Perturbation section provided below.

Method 400 also includes obtaining (408) a combination data point for a combination state (e.g., combination data point 139, as illustrated in FIGS. 1A, 2C, and 3D, obtained from measurements of cellular characteristics in instances of experimental conditions representative of a combination state 110). The combination data point includes the plurality of dimensions (e.g., the same number of dimensions as the corresponding baseline data point 133, perturbation data point 135, and compound data point 137), each respective dimension in the plurality of dimensions of the combination data point representing the measurement of central tendency of a different cellular characteristic, in the plurality of cellular characteristics (the same cellular characteristics that were measured for the corresponding baseline state, perturbation state, and compound state), determined across a corresponding plurality of combination aliquots of cells representing the combination state (e.g., referring to the hypothetical example with reference to FIG. 3B, the cellular characteristics are measured for each well 354-4 across the fourth row of multiwell plate 352, each of which contains an instance of an experimental condition representative of the combination state).

The combination state includes a third perturbation of the first cellular context in which (i) the expression of the gene is perturbed relative to the expression of the gene in the baseline state (as in the corresponding perturbation state) and (ii) the first cellular context is exposed to the compound (the same compound as was exposed to the compound state). As described above, with reference to the corresponding perturbation state, expression of the target gene may be perturbed in any number of fashions, e.g., siRNA knock-down with a single siRNA species, a plurality of siRNA species, or different siRNA species in difference instances of the experimental condition. However, it is desirable that the methodology used to perturb the target gene expression be the same as the methodology used in the perturbation state, such that any difference in the measured cellular characteristics, relative to the perturbation state, attributable to the interaction between the targeted gene and the compound, can more easily be identified. Similarly, as described herein with reference to the compound state, the concentration of the test compound may be selected based on various known or expected properties of the compound. However, it is desirable that the concentration of the test compound in the combination state be the same as the concentration of the test compound used in the compound state, such that any difference in the measured cellular characteristics, relative to the compound state, attributable to the interaction between the targeted gene and the compound, can more easily be identified.

Method 400 proceeds to a block 403 illustrated in FIG. 4C. Method 400 then includes featurizing the data points obtained above (e.g., baseline data point 133, perturbation data point 135, compound data point 137, and combination data point 139), to reduce the dimensionality of the data and improve upon any scarcity in the data, e.g., as described above with reference to step 140 in FIG. 1A. As described herein, the lower dimensionality of the featurized data point decreases the processing time required to analyze the data, thereby creating a more efficient processing system 200. Additionally, in some embodiments, featurizing the data reduces or removes multicollinearity between cellular characteristics, which are intercorrelations or inter-associations among independent variables in a data set. Multicollinerarity can cause problems during data analysis that result in models with high errors, e.g., due to poorly estimated partial regression coefficients during regression analysis. Accordingly, featurizing the data can both improve the speed of the process, as well as the results.

Method 400 includes featurizing (422) the baseline data point (e.g., baseline data point 133) by applying a dimension reduction model to the baseline data point, thereby generating a plurality of baseline feature values for the baseline data point. The plurality of baseline feature values define a baseline featurized vector (e.g., baseline feature values F_{B1} through F_{Bn} of baseline featurized data point 143) that has fewer dimensions than the corresponding data point (e.g., baseline data point 133).

Method 400 includes featurizing (424) the perturbation data point (e.g., perturbation data point 135) by applying the dimension reduction model (the same model as used to featurize baseline data point 133) to the perturbation data point, thereby generating a plurality of perturbation feature values for the perturbation data point. The plurality of perturbation feature values define a perturbation featurized vector (e.g., perturbation feature values F_{P1} through F_{Pn} of perturbation featurized data point 145) that has fewer dimensions than the corresponding data point (e.g., perturbation data point 135).

Method 400 includes featurizing (426) the compound data point (e.g., compound data point 137) by applying the dimension reduction model (the same model as used to featurize baseline data point 133 and perturbation data point 135) to the compound data point, thereby generating a plurality of compound feature values for the compound data point. The plurality of compound feature values define a compound featurized vector (e.g., compound feature values F_{D1} through F_{Dn} of compound featurized data point 147) that has fewer dimensions than the corresponding data point (e.g., compound data point 137).

Method 400 includes featurizing (428) the combination data point (e.g., combination data point 139) by applying the dimension reduction model (the same model as used to featurize baseline data point 133, perturbation data point 135, and compound data point 137) to the combination data point, thereby generating a plurality of combination feature values for the combination data point. The plurality of combination feature values define a combination featurized vector (e.g., combination feature values F_{C1} through F_{Cn} of combination featurized data point 149) that has fewer dimensions than the corresponding data point (e.g., combination data point 139).

Featurization of the cellular characteristics is performed by a dimensional reduction technique that uses a statistical feature selection or feature extraction procedure known in the art, for example, principal component analysis, non-negative matrix factorization, kernel PCA, graph-based kernel PCA, linear discriminant analysis, generalized discriminant analysis, and use of an autoencoder. This, in turn, reduces the computational burden of analyzing the data set by compressing the data in order to make the method more computationally efficient, e.g., by allowing the computer to apply an algorithm to the smaller dataset rather than the full dataset. Further information about featurization is described herein, e.g., in the Dimension Reduction section provided below.

In some embodiments, the dimension reduction model is a set of principal components (430) explaining variance across a training dataset including measurements of the plurality of cellular characteristics determined across a plurality of experimental states, where each experimental state in the plurality of experimental states includes a cellular context. For instance, in some embodiments, a set of principal components is learned against a training data set that includes measurements of the same cellular characteristics as used in method 400. In some embodiments, the training data set includes data derived from measurements of cellular characteristics from a plurality of instances of baseline states, e.g., unperturbed cell contexts. In some embodiments, the training data set includes data derived from measurements of cellular characteristics from a plurality of instances of perturbation states, e.g., cell contexts in which the expression of one or more target genes is perturbed, relative to a corresponding unperturbed baseline cellular context. In some embodiments, the training data set includes data derived from measurements of cellular characteristics from a plurality of instances of compound states, e.g., cell contexts which are exposed to one or more test compounds (e.g., candidate drugs, soluble factors, and/or toxins). In some embodiments, the training data set includes data derived from measurements of cellular characteristics from a plurality of instances of combination states, e.g., cell contexts in which the expression of one or more target genes is perturbed, relative to a corresponding unperturbed baseline cellular context, and is exposed to one or more test compounds (e.g., candidate drugs, soluble factors, and/or toxins). In some embodiments, the training data set includes data derived from measurements of cellular characteristics from a plurality of instances of baseline states and a plurality of instances of perturbation states and/or compound states. In some embodiments, the training data set includes data derived from measurements of cellular characteristics from a plurality of instances of baseline states, a plurality of instances of perturbation states, a plurality of instances of compound states, and a plurality of instances of combination states.

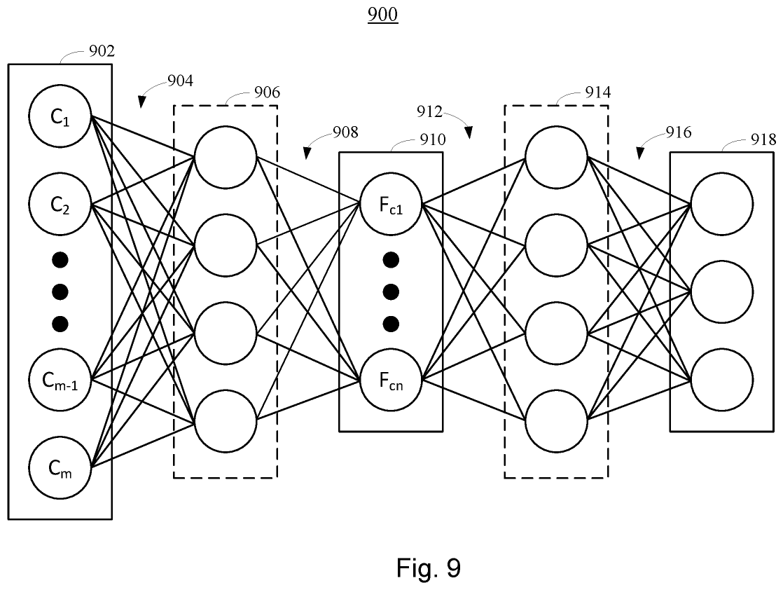

In some embodiments, the dimension reduction model makes use of a neural network (432), (e.g., as illustrated in FIG. 9) where the neural network includes: (i) an input layer comprising the plurality of dimensions (e.g., input layer 902), where the input layer receives the baseline data point (e.g., baseline data point 133), perturbation data point (e.g., perturbation data point 135), compound data point (e.g., compound data point 137), or combination data point (e.g., combination data point 139), and (ii) an embedding layer (e.g., embedding layer 910) that directly or indirectly receives output from the input layer. The embedding layer is associated with a plurality of weights (e.g., applied via connections 908) and, responsive to input of data into the neural network, produces an embedding layer (e.g., embedding layer 910) output having fewer dimensions than the plurality of dimensions (e.g., input layer 902, illustrated in FIG. 9, has m-dimensions, while embedding layer 910 has n-dimensions, where m>n). The plurality of weights (e.g., used in neural network 900) was trained against a training dataset including measurements of the plurality of cellular characteristics determined across a plurality of reference experimental states using a loss function, where each reference experimental state in the plurality of experimental states comprises an independent cellular context. For instance, in some embodiments, a neural network (e.g., neural network 900) is trained against a training data set that includes measurements of the same cellular characteristics as used in method 400.

In some embodiments, the training data set includes data derived from measurements of cellular characteristics from a plurality of instances of baseline states, e.g., unperturbed cell contexts. In some embodiments, the training data set includes data derived from measurements of cellular characteristics from a plurality of instances of perturbation states, e.g., cell contexts in which the expression of one or more target genes is perturbed, relative to a corresponding unperturbed baseline cellular context. In some embodiments, the training data set includes data derived from measurements of cellular characteristics from a plurality of instances of compound states, e.g., cell contexts which are exposed to one or more test compounds (e.g., candidate drugs, soluble factors, and/or toxins). In some embodiments, the training data set includes data derived from measurements of cellular characteristics from a plurality of instances of combination states, e.g., cell contexts in which the expression of one or more target genes is perturbed, relative to a corresponding unperturbed baseline cellular context, and is exposed to one or more test compounds (e.g., candidate drugs, soluble factors, and/or toxins). In some embodiments, the training data set includes data derived from measurements of cellular characteristics from a plurality of instances of baseline states and a plurality of instances of perturbation states and/or compound states. In some embodiments, the training data set includes data derived from measurements of cellular characteristics from a plurality of instances of baseline states, a plurality of instances of perturbation states, a plurality of instances of compound states, and a plurality of instances of combination states.

In some embodiments, e.g., referring to FIG. 9, neural network 900 includes an input layer 902 including a plurality of dimensions (e.g., the same number of dimensions as data points 133, 135, 137, and 139, where the input layer receives the baseline data point, perturbation data point, compound data point, or combination data point. For example, as illustrated in FIG. 9, each dimension of input layer 902 receives a term Cᵢ of combination data point 139 (e.g., as illustrated in FIG. 1A). Neural network 900 also includes embedding layer 910 linked to input layer 902 directly or indirectly. For instance, in some embodiments, neural network 900 includes one or more hidden layers 906 positioned between input layer 902 and embedding layer 910 (e.g., coupled via connections 904 and 908). In other embodiments, there are no hidden layers between input layer 902 and embedding layer 910, such that embedding layer 910 receives the output of input layer 902 directly. Embedding layer 910 includes fewer dimensions (e.g., n dimensions) than input layer 902 (e.g., m dimensions, where m>n). Neural network 900 also includes output layer 918 that directly or indirectly receives the output of embedding layer 910. For instance, in some embodiments, neural network 900 includes one or more hidden layers 914 positioned between embedding layer 910 and output layer 918 (e.g., coupled via connections 912 and 916). In other embodiments, there are no hidden layers between embedding layer 910 and output layer 918, such that output layer 918 receives the output of embedding layer 910 directly (e.g., via connections 916). In some embodiments, output layer 918 predicts an attribute of a test experimental state that includes a cellular context, responsive to loading a test data point that includes the plurality of dimensions and is representative of the test state (e.g., a data point of measures of central tendency of cellular characteristics measured across a plurality of instances of the test state). In some embodiments, the neural network is the neural network was trained against a training dataset comprising measurements of the plurality of cellular characteristics determined across a plurality of reference experimental states, wherein each reference experimental state in the plurality of experimental states comprises an independent cellular context, e.g., as described above. The portion of the neural network that serves as the dimension reduction model consists of the input layer (e.g., input layer 902), the embedding layer (e.g., embedding layer 910), and all hidden layers (e.g., optional hidden layer 906) linking the input layer to the embedding layer, such that the output of the embedding layer are the baseline feature values, perturbation feature values, compound feature values, or combination feature values (e.g., as illustrated in FIG. 9, each dimension of input layer 902 receives a term Cᵢ of combination data point 139 and each layer of embedding layer 910 outputs a term F_{ci} of combination state featurized vector 149).

Methods for training neural networks are generally known in the art, e.g., using back propagation techniques, such as stochastic gradient descent. Accordingly, in some embodiments, neural network is trained in a supervised fashion (434). In other embodiments, e.g., where the neural network is an autoencoder, the neural network is trained in an unsupervised fashion (434). For more information regarding artificial neural networks, see, for example, Abiodun O I, et al., Heliyon, 4(11):e00938 (2018), the content of which is incorporated herein by reference.

With reference to FIG. 4D, method 400 then includes determining (438) whether the compound (the compound included in compound state 108 and combination state 110) interacts with the gene (the gene whose expression is perturbed in perturbation state 106 and combination state 110) by using the plurality of baseline feature values (e.g., baseline featurized data point 143), the plurality of perturbation feature values (e.g., perturbation featurized data point 145), the plurality of compound feature values (e.g., compound featurized data point 147), and the plurality of combination feature values (e.g., combination featurized data point 149) to resolve whether the combination of the gene and the compound has a threshold interaction effect on one or more cellular characteristic in the plurality of cellular characteristics (e.g., whether the change in cellular characteristics in the combination state, relative to the cellular characteristics in the baseline state, is significantly more or less than would be expected from the combination of changes, relative to the baseline state, observed in the perturbation state and the compound state). The compound interacts with the gene when the combination of the gene and the compound has a threshold effect on one or more cellular characteristic in the plurality of cellular characteristics. The compound does not interact with the gene when the combination of the gene and the compound does not have a threshold effect on one or more cellular characteristic in the plurality of cellular characteristics.

In some embodiments, a statistical hypothesis test, using the feature values derived from the cell assay data, is performed (440) to determine whether the compound interacts with the gene. In some embodiments, the statistical hypothesis test is performed (440) against at least the plurality of combination feature values using a null hypothesis that the compound does not interact with the gene. In some embodiments, the statistical hypothesis test is a two-way ANOVA performed (442) against each respective combination feature value in the plurality of combination feature values, thereby generating a corresponding p-value for each respective combination feature value in the plurality of combination feature values. For instance, in some embodiments, a two-way ANOVA is performed against each feature F_{ci} of combination featurized data set 149, using corresponding features F_{Bi} of baseline featurized data set 143, F_{Pi} of perturbation featurized data set 145, and F_{Bi} of compound featurized data set 147, thereby generating a corresponding p-value 159 for each feature F_{ci} of combination featurized data set 149.

In some embodiments, determining whether the compound interacts with a gene includes generating (444) a test statistic X² by combining the corresponding p-values (e.g., p-values 159) for each respective combination feature value (e.g., F_{ci}) in the plurality of combination feature values (e.g., featurized data set 149). Methods of meta-analysis combining p-values are known in the art and include, for example, Fischer's method, Pearson's method, George's method, Edgington's method, Stouffer's method, Tippett's method, use of a Beta distribution, use of a truncated gamma distribution, and use of other general distribution functions. For more information regarding methods for combining p-values, and rationales for choosing a particular meta-analysis method, see, for example, Heard N A, “Choosing Between Methods of Combining p-values,” arXiv:1707.06897v4 [stat.ME] 14 Dec. 2017, the content of which is hereby incorporated by reference.

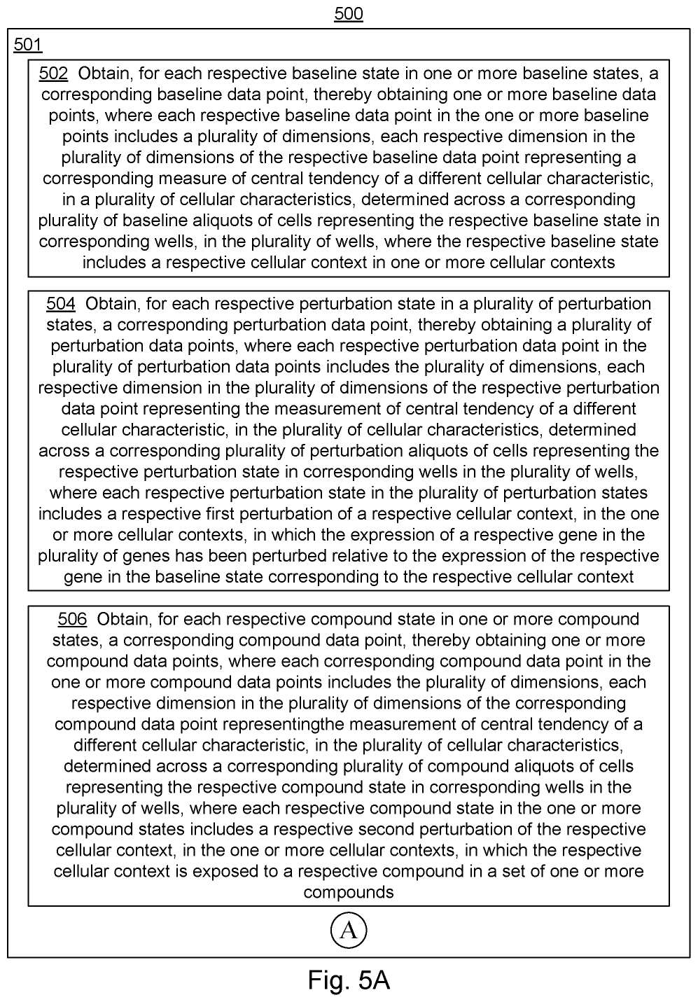

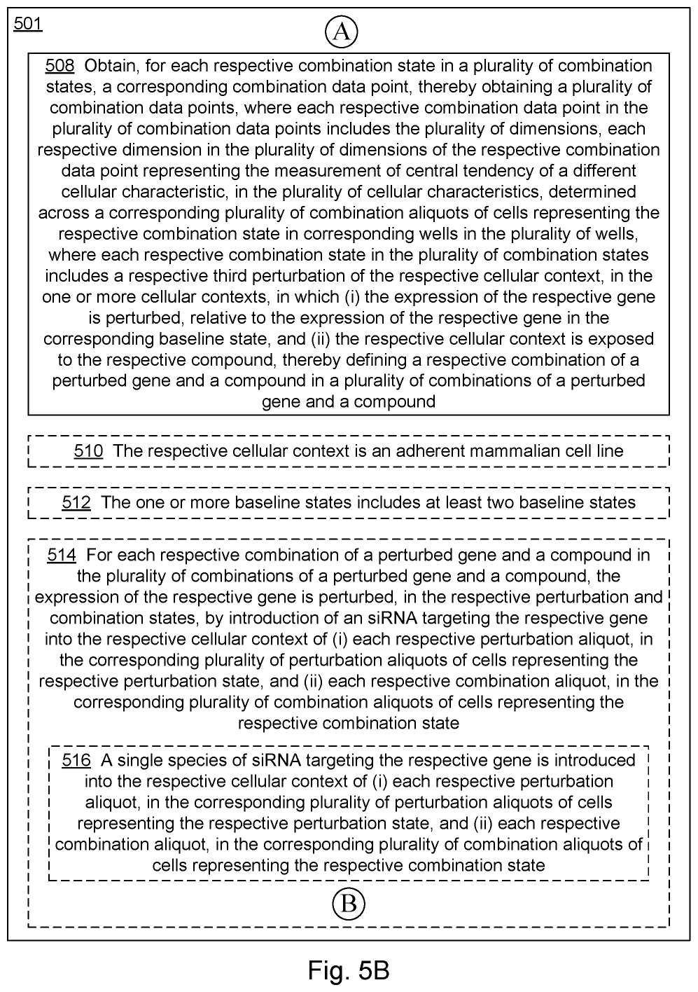

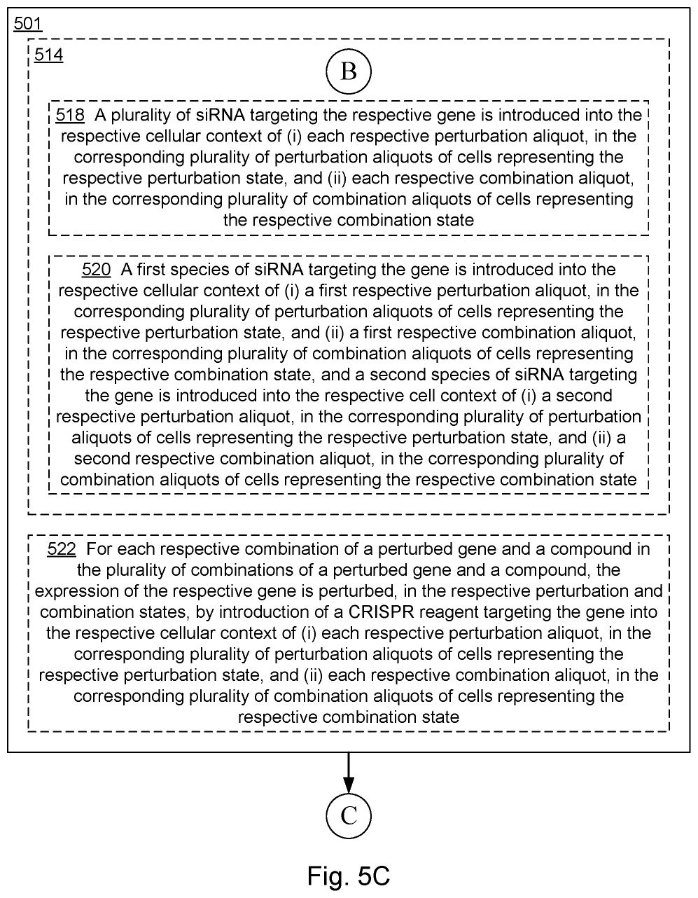

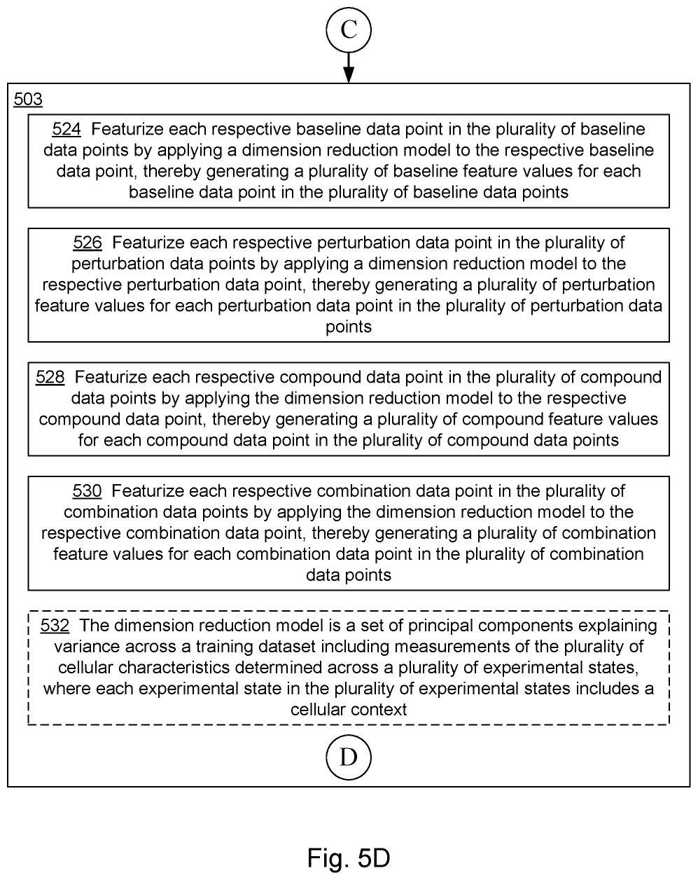

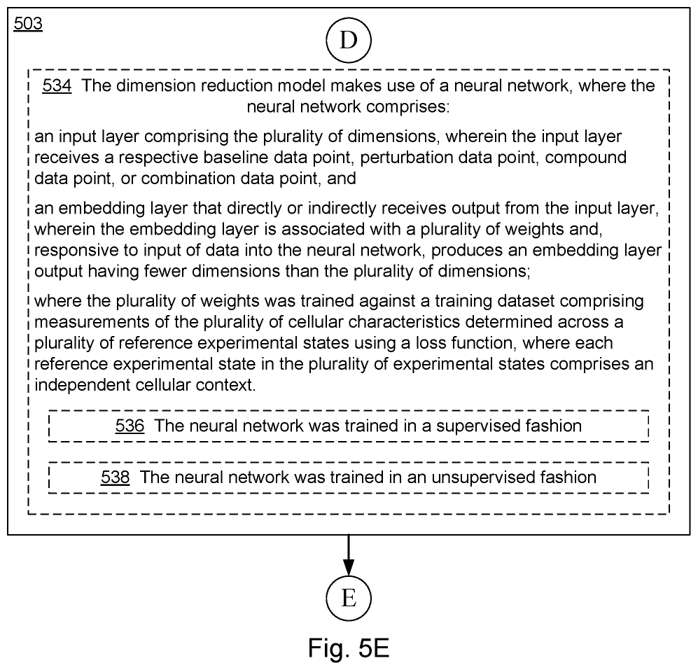

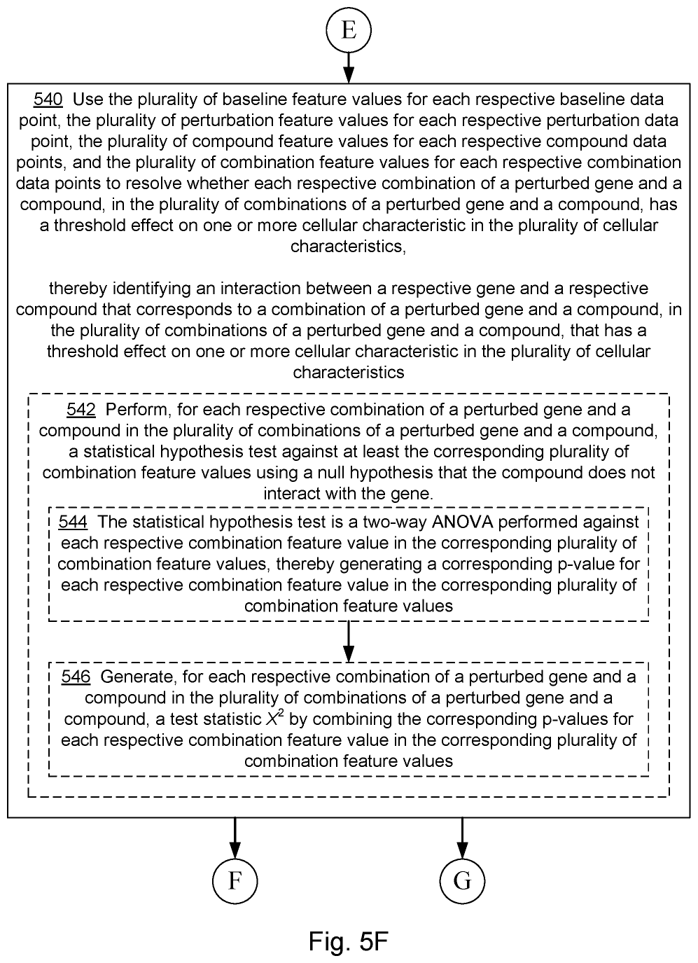

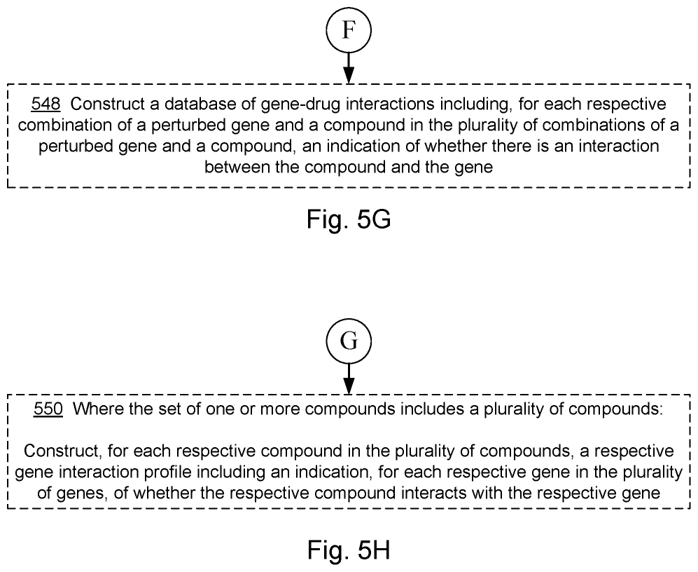

In a related aspect, the disclosure also provides a method 500 for identifying interactions between one or more compounds and a plurality of genes, e.g., in an interaction screen performed with a plurality of perturbation states. As described for method 400 above, and illustrated in FIG. 5, method 500 includes analyzing pairwise interactions between respective compounds, e.g., a candidate drug, soluble factor, or toxin, and perturbed genes. In some embodiments, method 500 is performed such that each compound is queried against at least 10 different perturbed genes. In some embodiments, method 500 is performed with at least 25 different perturbed genes, or at least 50, 100, 200, 300, 400, 500, 600, 700, 800, 900, 1000, 5000, or more different perturbed genes.

Method 500 begins with a block 501 which is illustrated in FIGS. 5A and 5B. Method 500 includes obtaining (502) for each respective baseline state in one or more baseline states, a corresponding baseline data point (e.g., baseline data point 133, as illustrated in FIGS. 1A, 2C, and 3D, obtained from measurements of cellular characteristics in instances of experimental conditions representative of a baseline state 104), thereby obtaining one or more baseline data points, where each respective baseline data point in the one or more baseline points includes a plurality of dimensions, each respective dimension in the plurality of dimensions of the respective baseline data point representing a corresponding measure of central tendency of a different cellular characteristic, in a plurality of cellular characteristics, determined across a corresponding plurality of baseline aliquots of cells representing the respective baseline state in corresponding wells, in the plurality of wells, where the respective baseline state includes a respective cellular context in one or more cellular contexts. The one or more baseline states may include two baseline states (512).

For instance, each baseline cellular characteristic 113 is measured in each instance of an experimental condition representative of baseline state 104 in the assay (e.g., measurements 113-1-1 through 113-1-16 of the same characteristic are obtained from wells 354-1-1 through 354-1-16, respectively, in FIG. 3B) and then a measure of central tendency is determined across each of the measurements (e.g., the mean of measurements 113-1-1 through 113-1-16 of the first characteristic

$$( {\sum }_{i = 16}^{1} ( \frac{113 - 1 - 1 - 1 - i}{16} ) ) ,$$

as illustrated in FIG. 3D). The measures of central tendency for each of the characteristics representative of the experimental state are then concatenated into a baseline data point (e.g., data point 133) for the respective cellular context. In some embodiments, the measure of central tendency is an arithmetic mean, weighted mean, midrange, midhinge, trimean, geometric mean, geometric median, Winsorized mean, median, or mode of the cellular characteristic.

In some embodiments, each of the cellular characteristics is an optically-measureable characteristic. In other embodiments, at least one cellular characteristic in the plurality of cellular characteristics is measured using a non-optical measurement. Non-limiting examples of optically-measureable and non-optically measureable cellular characteristic are described herein, e.g., in the Cellular Characteristic sections provided below.

In some embodiments, each of the baseline aliquots of cells include a single cell type, e.g., a single type of mammalian cell. In some embodiments, each of the baseline aliquots of cells includes the same mixture of different cell types, e.g., a co-culture of two different mammalian cells. In yet other embodiments, the plurality of baseline aliquots of cells includes different cellular contexts in different instances of the experimental condition that is representative of the baseline state. As described in further detail below, in some embodiments, measurements of the same cellular characteristic are acquired from different cell types in order to account for changes in the characteristic, e.g., upon perturbation of gene expression, exposure to a compound, and/or both, that are specific to one cell type. For instance, referring back to the hypothetical experiment described above with reference to FIGS. 3B-3D, in some embodiments, each baseline experimental condition in wells 354-1-1 to 354-1-16 contains a different cell type, or every two wells across the first row contain a different cell type, or every three wells across the first row contain a different cell type, etc.

In one embodiment, the respective cellular context is a mammalian cell line. In one embodiment, the respective cellular context is an adherent mammalian cell line (510). In some embodiments, the respective cellular context is a human cell. In some embodiments, the respective cellular context is a primary human cell. Non-limiting examples of cell types useful for the methods provided herein are described herein, e.g., in the Cell Contexts section provided below.

Method 500 also includes obtaining (504) for each respective perturbation state in a plurality of perturbation states, a perturbation data point (e.g., perturbation data point 135, as illustrated in FIGS. 1A, 2C, and 3D, obtained from measurements of cellular characteristics in instances of experimental conditions representative of a perturbation state 106), thereby obtaining a plurality of perturbation data points, where each respective perturbation data point in the plurality of perturbation data points includes the plurality of dimensions, each respective dimension in the plurality of dimensions of the respective perturbation data point representing the measurement of central tendency of a different cellular characteristic, in the plurality of cellular characteristics, determined across a corresponding plurality of perturbation aliquots of cells representing the respective perturbation state in corresponding wells in the plurality of wells, where each respective perturbation state in the plurality of perturbation states includes a respective first perturbation of a respective cellular context, in the one or more cellular contexts, in which the expression of a respective gene in the plurality of genes has been perturbed relative to the expression of the respective gene in the baseline state corresponding to the respective cellular context The perturbation data point includes the plurality of dimensions (e.g., the same number of dimensions as the corresponding baseline data point 133), each respective dimension in the plurality of dimensions of the perturbation data point representing the measurement of central tendency of a different cellular characteristic, e.g., in the plurality of cellular characteristics (the same cellular characteristics that were measured for the baseline state), determined across a plurality of perturbation aliquots of cells representing the perturbation state (e.g., referring to the hypothetical example with reference to FIG. 3B, the cellular characteristics are measured for each well 354-2 across the second row of multiwell plate 352, each of which contains an instance of an experimental condition representative of the perturbation state).

The perturbation state includes a first perturbation of the first cellular context in which the expression of a gene is perturbed relative to the expression of the gene in the baseline state. That is, the background for the cellular context(s) used in the perturbation experimental conditions is the same as the cellular context used in the background experimental conditions. However, the expression of a target gene in the background cellular context is perturbed relative to the expression of the target gene in the baseline cellular contexts. As described with reference to the baseline state above, in some embodiments, the same cellular context is used in each of the perturbation experimental conditions (e.g., when the same cellular context is used in each of the baseline experimental conditions). In other embodiments, different cellular contexts are used in different instances of the perturbation experimental conditions (e.g., when different cellular contexts are used in different instances of the baseline experimental conditions). The point is that it is advantageous to use as close to the same cellular background, as possible, in the experimental conditions corresponding to the baseline state and the experimental conditions corresponding to the perturbation state, so that differences in the cellular characteristics of the perturbation state, relative to the baseline state, can be confidently attributable to the perturbation of the target gene.

As outlined above, because the methods provided herein are not tied to any particular cellular dysfunction or disease model, the expression of any gene in the cellular context may be perturbed, to identify interactions with that gene and a second biological agent (e.g., another gene, a candidate drug compound, a soluble factor, or a toxin). Moreover, unlike conventional synthetic lethality assays used to identify interactions between biological agents, interactions between agents that do not cause drastic changes in a cellular feature (e.g., like apoptosis) can still be identified, broadening the utility of the methods described herein beyond conventional methodologies for identifying interactions in a complex biological system.

In some embodiments, for each respective combination of a perturbed gene and a compound in the plurality of combinations of a perturbed gene and a compound, the expression of the target gene is perturbed, in the perturbation state, by introduction of an siRNA targeting the gene into the first cellular context of the plurality of perturbation aliquots of cells representing the perturbation state (514). For instance, the cells included in wells 354-2-1 through 354-2-16, representing the perturbation state in the hypothetical example illustrated in FIG. 3B, are the same cells included in wells 354-1-1 through 354-1-16, representing the baseline state, except that one or more siRNA directed to the target gene has been introduced into the cells included in wells 354-2-1 through 354-2-16, but not into the cells included in wells 354-1-1 through 354-1-16.

In some embodiments, a single species of siRNA targeting the gene (e.g., siRNA with a single, defined sequence) is introduced into the first cellular context of each respective perturbation aliquot, in the plurality of perturbation aliquots of cells representing the perturbation state (516). That is, in some embodiments, for every gene that interaction data is being queried, a single siRNA sequence is used in each instance of the perturbation state. In some embodiments, a plurality of siRNA targeting the gene is introduced into the first cellular context of each respective perturbation aliquot, in the plurality of perturbation aliquots of cells representing the perturbation state (518). That is, in some embodiments, multiple siRNA sequences are used to perturb the expression of the target gene.

In yet other embodiments, a first species of siRNA targeting the gene is introduced into the first cell context of a first respective perturbation aliquot, in the plurality of perturbation aliquots of cells representing the perturbation state, and a second species of siRNA targeting the gene is introduced into the first cell context of a second respective perturbation aliquot, in the plurality of perturbation aliquots of cells representing the perturbation state (520). That is, in some embodiments, different siRNA molecules that target a different portion (sequence) of the target gene are used in different instances of the perturbation state. For instance, referring to the hypothetical example illustrated in FIG. 3B, a first siRNA directed to a targeted gene is introduced into cells used in well 354-2-1 of plate 352, and a second siRNA directed to a different sequence in the targeted gene is introduced into cells used in well 354-2-2 (or every other well, every third well, etc,), such that the characteristics represented in the resulting perturbation data point 115 are measures of central tendencies of the characteristic measured across cells in which the targeted gene is perturbed using difference siRNA species. It is known that some siRNA perturb the expression of genes other than the target gene. These off-target affects, depending on the extent to which they affect the expression of the off-target gene, may significantly affect the cellular characteristics measured in the assays described herein. By using a plurality of different siRNA sequences directed to the target gene, changes in the cellular characteristics from any particular off-target effect can be averaged out, since different off-target genes would be expected to be affected by different siRNA sequences.

In some embodiments, the expression of the gene is perturbed, in the perturbation state, by introduction of a CRISPR reagent targeting the gene into the first cellular context of the plurality of perturbation aliquots of cells representing the perturbation state (522). For instance, the cells included in wells 354-2-1 through 354-2-16, representing the perturbation state in the hypothetical example illustrated in FIG. 3B, are the same cells included in wells 354-1-1 through 354-1-16, representing the baseline state, except that the target gene has been altered by one or more CRISPR reagents in the cells included in wells 354-2-1 through 354-2-16, but not into the cells included in wells 354-1-1 through 354-1-16. More details with respect to methods for perturbing gene expression are described herein, e.g., in the Gene Expression Perturbation section provided below.

Method 500 also includes obtaining (506) a compound data point for a compound state for each respective compound state in one or more compound states, a corresponding compound data point (e.g., compound data point 137, as illustrated in FIGS. 1A, 2C, and 3D, obtained from measurements of cellular characteristics in instances of experimental conditions representative of a compound state 108), thereby obtaining one or more compound data points, where each corresponding compound data point in the one or more compound data points includes the plurality of dimensions, each respective dimension in the plurality of dimensions of the corresponding compound data point representing the measurement of central tendency of a different cellular characteristic, in the plurality of cellular characteristics, determined across a corresponding plurality of compound aliquots of cells representing the respective compound state in corresponding wells in the plurality of wells, where each respective compound state in the one or more compound states includes a respective second perturbation of the respective cellular context, in the one or more cellular contexts, in which the respective cellular context is exposed to a respective compound in a set of one or more compounds. The compound data point includes the plurality of dimensions (e.g., the same number of dimensions as the corresponding baseline data point 133 and perturbation data point 135), each respective dimension in the plurality of dimensions of the compound data point representing the measurement of central tendency of a different cellular characteristic, in the plurality of cellular characteristics (the same cellular characteristics that were measured for the corresponding baseline state and perturbation state), determined across a plurality of compound aliquots of cells representing the compound state (e.g., referring to the hypothetical example with reference to FIG. 3B, the cellular characteristics are measured for each well 354-3 across the third row of multiwell plate 352, each of which contains an instance of an experimental condition representative of the compound state).

The compound state includes a second perturbation of the first cellular context in which the first cellular context is exposed to a compound. That is, the cellular context(s) used in the compound experimental conditions is the same as the cellular context used in the background experimental conditions, as well as the basis for the cellular context used in the corresponding perturbation experimental conditions. However, the cellular context is exposed to a test compound, e.g., a candidate drug, a soluble factor, or a toxin. In some embodiments, the compound is a candidate drug compound, such that the method is for identifying an interaction between a gene and a candidate drug compound. In some embodiments, the compound is a soluble factor, such that the method is for identifying an interaction between a gene and a soluble factor. In some embodiments, the compound is a toxin, such that the method is for identifying an interaction between a gene and a toxin. More details with respect to compounds useful for method 500 are described herein, e.g., in Compound Perturbation section provided below.

Method 500 also includes obtaining (508 for each respective combination state in a plurality of combination states, a corresponding combination data point (e.g., combination data point 139, as illustrated in FIGS. 1A, 2C, and 3D, obtained from measurements of cellular characteristics in instances of experimental conditions representative of a combination state 110), thereby obtaining a plurality of combination data points, where each respective combination data point in the plurality of combination data points includes the plurality of dimensions, each respective dimension in the plurality of dimensions of the respective combination data point representing the measurement of central tendency of a different cellular characteristic, in the plurality of cellular characteristics, determined across a corresponding plurality of combination aliquots of cells representing the respective combination state in corresponding wells in the plurality of wells. The combination data point includes the plurality of dimensions (e.g., the same number of dimensions as the corresponding baseline data point 133, perturbation data point 135, and compound data point 137), each respective dimension in the plurality of dimensions of the combination data point representing the measurement of central tendency of a different cellular characteristic, in the plurality of cellular characteristics (the same cellular characteristics that were measured for the corresponding baseline state, perturbation state, and compound state), determined across a corresponding plurality of combination aliquots of cells representing the combination state (e.g., referring to the hypothetical example with reference to FIG. 3B, the cellular characteristics are measured for each well 354-4 across the fourth row of multiwell plate 352, each of which contains an instance of an experimental condition representative of the combination state).

The respective combination state in the plurality of combination state includes a third perturbation of the first cellular context in which (i) the expression of the gene is perturbed relative to the expression of the gene in the baseline state (as in the corresponding perturbation state) and (ii) the first cellular context is exposed to the compound (the same compound as was exposed to the compound state). As described above, with reference to the corresponding perturbation state, expression of the target gene may be perturbed in any number of fashions, e.g., siRNA knock-down with a single siRNA species, a plurality of siRNA species, or different siRNA species in difference instances of the experimental condition. However, it is desirable that the methodology used to perturb the target gene expression be the same as the methodology used in the perturbation state, such that any difference in the measured cellular characteristics, relative to the perturbation state, attributable to the interaction between the targeted gene and the compound, can more easily be identified. Similarly, as described herein with reference to the compound state, the concentration of the test compound may be selected based on various known or expected properties of the compound. However, it is desirable that the concentration of the test compound in the combination state be the same as the concentration of the test compound used in the compound state, such that any difference in the measured cellular characteristics, relative to the compound state, attributable to the interaction between the targeted gene and the compound, can more easily be identified.

Method 500 proceeds to a block 503 illustrated in FIG. 5C. Method 500 then includes featurizing the data points obtained above (e.g., baseline data point 133, perturbation data point 135, compound data point 137, and combination data point 139), to reduce the dimensionality of the data and improve upon any scarcity in the data, e.g., as described above with reference to step 140 in FIG. 1A. As described herein, the lower dimensionality of the featurized data point decreases the processing time required to analyze the data, thereby creating a more efficient processing system 200. Additionally, in some embodiments, featurizing the data reduces or removes multicollinearity between cellular characteristics, which are intercorrelations or inter-associations among independent variables in a data set. Multicollinerarity can cause problems during data analysis that result in models with high errors, e.g., due to poorly estimated partial regression coefficients during regression analysis. Accordingly, featurizing the data can both improve the speed of the process, as well as the results.

With reference to FIG. 5D, method 500 includes featurizing (524) each respective baseline data point in the plurality of baseline data points (e.g., baseline data point 133) by applying a dimension reduction model to the respective baseline data point, thereby generating a plurality of baseline feature values for each baseline data point in the plurality of baseline data points. The plurality of baseline feature values for a respective baseline data point define a baseline featurized vector (e.g., baseline feature values F_{B1} through F_{Bn} of baseline featurized data point 143) that has fewer dimensions than the corresponding data point (e.g., baseline data point 133).

Method 500 includes featurizing (526) each respective perturbation data point in the plurality of perturbation data points (e.g., perturbation data point 135) by applying the dimension reduction model (the same model as used to featurize baseline data point 133) to the respective perturbation data point, thereby generating a plurality of perturbation feature values for each perturbation data point in the plurality of perturbation data points. The plurality of perturbation feature values for a respective perturbation data point define a perturbation featurized vector (e.g., perturbation feature values F_{P1} through F_{Pn} of perturbation featurized data point 145) that has fewer dimensions than the corresponding data point (e.g., perturbation data point 135).

Method 500 includes featurizing (528) each respective compound data point in the plurality of compound data points (e.g., compound data point 137) by applying the dimension reduction model (the same model as used to featurize baseline data point 133 and perturbation data point 135) to the respective compound data point, thereby generating a plurality of compound feature values each compound data point in the plurality of compound data points. The plurality of compound feature values for a respective compound data point define a compound featurized vector (e.g., compound feature values F_{D1} through F_{Dn} of compound featurized data point 147) that has fewer dimensions than the corresponding data point (e.g., compound data point 137).

Method 500 includes featurizing (530) each respective combination data point of the plurality of combination data points (e.g., combination data point 139) by applying the dimension reduction model (the same model as used to featurize baseline data point 133, perturbation data point 135, and compound data point 137) to the respective combination data point, thereby generating a plurality of combination feature values for each combination data point of the plurality of combination data points. The plurality of combination feature values for a respective combination data point define a combination featurized vector (e.g., combination feature values F_{C1} through F_{Cn} of combination featurized data point 149) that has fewer dimensions than the corresponding data point (e.g., combination data point 139).

Featurization of the cellular characteristics is performed by a dimensional reduction technique that uses a statistical feature selection or feature extraction procedure known in the art, for example, principal component analysis, non-negative matrix factorization, kernel PCA, graph-based kernel PCA, linear discriminant analysis, generalized discriminant analysis, and use of an autoencoder. This, in turn, reduces the computational burden of analyzing the data set by compressing the data in order to make the method more computationally efficient, e.g., by allowing the computer to apply an algorithm to the smaller dataset rather than the full dataset. Further information about featurization is described herein, e.g., in the Dimension Reduction section provided below.

In some embodiments, the dimension reduction model is a set of principal components (532) explaining variance across a training dataset including measurements of the plurality of cellular characteristics determined across a plurality of experimental states, where each experimental state in the plurality of experimental states includes a cellular context. For instance, in some embodiments, a set of principal components is learned against a training data set that includes measurements of the same cellular characteristics as used in method 500. In some embodiments, the training data set includes data derived from measurements of cellular characteristics from a plurality of instances of baseline states, e.g., unperturbed cell contexts. In some embodiments, the training data set includes data derived from measurements of cellular characteristics from a plurality of instances of perturbation states, e.g., cell contexts in which the expression of one or more target genes is perturbed, relative to a corresponding unperturbed baseline cellular context. In some embodiments, the training data set includes data derived from measurements of cellular characteristics from a plurality of instances of compound states, e.g., cell contexts which are exposed to one or more test compounds (e.g., candidate drugs, soluble factors, and/or toxins). In some embodiments, the training data set includes data derived from measurements of cellular characteristics from a plurality of instances of combination states, e.g., cell contexts in which the expression of one or more target genes is perturbed, relative to a corresponding unperturbed baseline cellular context, and is exposed to one or more test compounds (e.g., candidate drugs, soluble factors, and/or toxins). In some embodiments, the training data set includes data derived from measurements of cellular characteristics from a plurality of instances of baseline states and a plurality of instances of perturbation states and/or compound states. In some embodiments, the training data set includes data derived from measurements of cellular characteristics from a plurality of instances of baseline states, a plurality of instances of perturbation states, a plurality of instances of compound states, and a plurality of instances of combination states.

With reference to FIG. 5E, in some embodiments, the dimension reduction model makes use of a neural network (534), (e.g., as illustrated in FIG. 9) where the neural network includes: (i) an input layer comprising the plurality of dimensions (e.g., input layer 902), where the input layer receives a respective baseline data point (e.g., baseline data point 133), perturbation data point (e.g., perturbation data point 135), compound data point (e.g., compound data point 137), or combination data point (e.g., combination data point 139), and (ii) an embedding layer (e.g., embedding layer 910) that directly or indirectly receives output from the input layer. The embedding layer is associated with a plurality of weights (e.g., applied via connections 908) and, responsive to input of data into the neural network, produces an embedding layer (e.g., embedding layer 910) output having fewer dimensions than the plurality of dimensions (e.g., input layer 902, illustrated in FIG. 9, has m-dimensions, while embedding layer 910 has n-dimensions, where m>n). The plurality of weights (e.g., used in neural network 900) was trained against a training dataset including measurements of the plurality of cellular characteristics determined across a plurality of reference experimental states using a loss function, where each reference experimental state in the plurality of experimental states comprises an independent cellular context. For instance, in some embodiments, a neural network (e.g., neural network 900) is trained against a training data set that includes measurements of the same cellular characteristics as used in method 500.

In some embodiments, the training data set includes data derived from measurements of cellular characteristics from a plurality of instances of baseline states, e.g., unperturbed cell contexts. In some embodiments, the training data set includes data derived from measurements of cellular characteristics from a plurality of instances of perturbation states, e.g., cell contexts in which the expression of one or more target genes is perturbed, relative to a corresponding unperturbed baseline cellular context. In some embodiments, the training data set includes data derived from measurements of cellular characteristics from a plurality of instances of compound states, e.g., cell contexts which are exposed to one or more test compounds (e.g., candidate drugs, soluble factors, and/or toxins). In some embodiments, the training data set includes data derived from measurements of cellular characteristics from a plurality of instances of combination states, e.g., cell contexts in which the expression of one or more target genes is perturbed, relative to a corresponding unperturbed baseline cellular context, and is exposed to one or more test compounds (e.g., candidate drugs, soluble factors, and/or toxins). In some embodiments, the training data set includes data derived from measurements of cellular characteristics from a plurality of instances of baseline states and a plurality of instances of perturbation states and/or compound states. In some embodiments, the training data set includes data derived from measurements of cellular characteristics from a plurality of instances of baseline states, a plurality of instances of perturbation states, a plurality of instances of compound states, and a plurality of instances of combination states.

In some embodiments, e.g., referring to FIG. 9, neural network 900 includes an input layer 902 including a plurality of dimensions (e.g., the same number of dimensions as data points 133, 135, 137, and 139, where the input layer receives the baseline data point, perturbation data point, compound data point, or combination data point. For example, as illustrated in FIG. 9, each dimension of input layer 902 receives a term Cᵢ of combination data point 139 (e.g., as illustrated in FIG. 1A). Neural network 900 also includes embedding layer 910 linked to input layer 902 directly or indirectly. For instance, in some embodiments, neural network 900 includes one or more hidden layers 906 positioned between input layer 902 and embedding layer 910 (e.g., coupled via connections 904 and 908). In other embodiments, there are no hidden layers between input layer 902 and embedding layer 910, such that embedding layer 910 receives the output of input layer 902 directly. Embedding layer 910 includes fewer dimensions (e.g., n dimensions) than input layer 902 (e.g., m dimensions, where m>n). Neural network 900 also includes output layer 918 that directly or indirectly receives the output of embedding layer 910. For instance, in some embodiments, neural network 900 includes one or more hidden layers 914 positioned between embedding layer 910 and output layer 918 (e.g., coupled via connections 912 and 916). In other embodiments, there are no hidden layers between embedding layer 910 and output layer 918, such that output layer receives the output of embedding layer 910 directly (e.g., via connections 916). In some embodiments, output layer 918 predicts an attribute of a test experimental state that includes a cellular context, responsive to loading a test data point that includes the plurality of dimensions and is representative of the test state (e.g., a data point of measures of central tendency of cellular characteristics measured across a plurality of instances of the test state). In some embodiments, the neural network is the neural network was trained against a training dataset comprising measurements of the plurality of cellular characteristics determined across a plurality of reference experimental states, wherein each reference experimental state in the plurality of experimental states comprises an independent cellular context, e.g., as described above. The portion of the neural network that serves as the dimension reduction model consists of the input layer (e.g., input layer 902), the embedding layer (e.g., embedding layer 910), and all hidden layers (e.g., optional hidden layer 906) linking the input layer to the embedding layer, such that the output of the embedding layer are the baseline feature values, perturbation feature values, compound feature values, or combination feature values (e.g., as illustrated in FIG. 9, each dimension of input layer 902 receives a term Cᵢ of combination data point 139 and each layer of embedding layer 910 outputs a term F_{ci} of combination state featurized vector 149).

Methods for training neural networks are generally known in the art, e.g., using back propagation techniques, such as stochastic gradient descent. Accordingly, in some embodiments, neural network is trained in a supervised fashion (536). In other embodiments, e.g., where the neural network is an autoencoder, the neural network is trained in an unsupervised fashion (538). For more information regarding artificial neural networks, see, for example, Abiodun O I, et al., Heliyon, 4(11):e00938 (2018), the content of which is incorporated herein by reference.

With reference to FIG. 5F, method 500 then includes using (540) the plurality of baseline feature values (e.g., baseline featurized data point 143) for each respective baseline data point, the plurality of perturbation feature values (e.g., perturbation featurized data point 145) for each respective perturbation data point, the plurality of compound feature values (e.g., compound featurized data point 147) for each respective compound data points, and the plurality of combination feature values (e.g., combination featurized data point 149) for each respective combination data points to resolve whether each respective combination of a perturbed gene (the gene whose expression is perturbed in perturbation state 106 and combination state 110) and a compound (the compound included in compound state 108 and combination state 110), in the plurality of combinations of a perturbed gene and a compound, has a threshold effect on one or more cellular characteristic in the plurality of cellular characteristics, thereby identifying an interaction between a respective gene and a respective compound that corresponds to a combination of a perturbed gene and a compound, in the plurality of combinations of a perturbed gene and a compound, that has a threshold effect on one or more cellular characteristic in the plurality of cellular characteristics.

In some embodiments, for each respective combination of a perturbed gene and a compound in the plurality of combinations of a perturbed gene and a compound, a statistical hypothesis test is performed (542) against at least the corresponding plurality of combination feature values using a null hypothesis that the compound does not interact with the gene. In some embodiments, the statistical hypothesis test is a two-way ANOVA performed (544) against each respective combination feature value in the corresponding plurality of combination feature values, thereby generating a corresponding p-value for each respective combination feature value in the plurality of combination feature values.

In some embodiments, determining whether the compound interacts with a gene includes generating (546) for each respective combination of a perturbed gene and a compound in the plurality of combinations of a perturbed gene and a compound, a test statistic X² by combining the corresponding p-values (e.g., p-values 159) for each respective combination feature value (e.g., F_{ci}) in the corresponding plurality of combination feature values. Methods of meta-analysis combining p-values are known in the art and include, for example, Fischer's method, Pearson's method, George's method, Edgington's method, Stouffer's method, Tippett's method, use of a Beta distribution, use of a truncated gamma distribution, and use of other general distribution functions. For more information regarding methods for combining p-values, and rationales for choosing a particular meta-analysis method, see, for example, Heard N A, “Choosing Between Methods of Combining p-values,” arXiv:1707.06897v4 [stat.ME] 14 Dec. 2017, the content of which is hereby incorporated by reference.

With reference to FIG. 5G, in some embodiments, a database of gene-drug interactions is constructed (548) including, for each respective combination of a perturbed gene and a compound in the plurality of combinations of a perturbed gene and a compound, an indication of whether there is an interaction between the compound and the gene.

In some embodiments, the methods described herein further include constructing a database of compound-gene interactions including, for each respective combination of a compound and a gene, an indication of whether the first perturbation and the compound interacts with the gene.

The database of gene-drug (i.e., compound) interactions described above is used, in some embodiments, in a method for identifying a compound of therapeutic interest for a disease state associated with aberrant function of a gene or associated gene product. The method includes querying a database of gene-compound interactions, for a compound associated with an indication of an interaction between the compound and the gene, thereby identifying a compound of therapeutic interest for the disease state.

With reference to FIG. 5H, in some embodiments, where the set of one or more compounds includes a plurality of compounds: for each respective compound in the plurality of compounds, a respective gene interaction profile is constructed (550) including an indication, for each respective gene in the plurality of genes, of whether the respective compound interacts with the respective gene.

The gene interaction profile described above is used, in some embodiments, in a method for identifying a mechanism of action for a test compound. The method includes comparing a gene interaction profile for a test compound to a plurality of annotated gene interaction profiles, where each respective annotated gene interaction profile in the plurality of annotated interaction profiles is for a corresponding compound, in a plurality of corresponding compounds, having a known mechanisms of action.

The gene interaction profile described above is used, in some embodiments, in a method for identifying a polypharmacological effect of a test compound of interest. The method includes querying a gene interaction profile for the test compound for indications that the test compound interacts with a plurality of genes that are each associated with a same physiological disorder, thereby identifying a polypharmacological effect of the test compound for a physiological disorder when the gene interaction profile for the test compound includes indications that the test compound interacts with at least two genes associated with the physiological disorder.

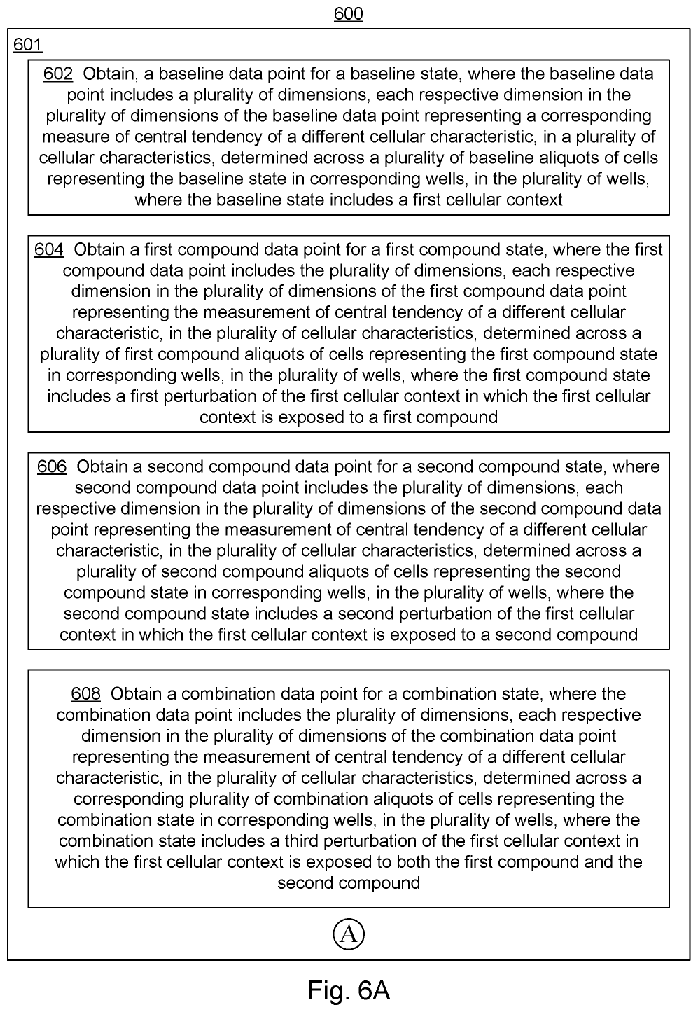

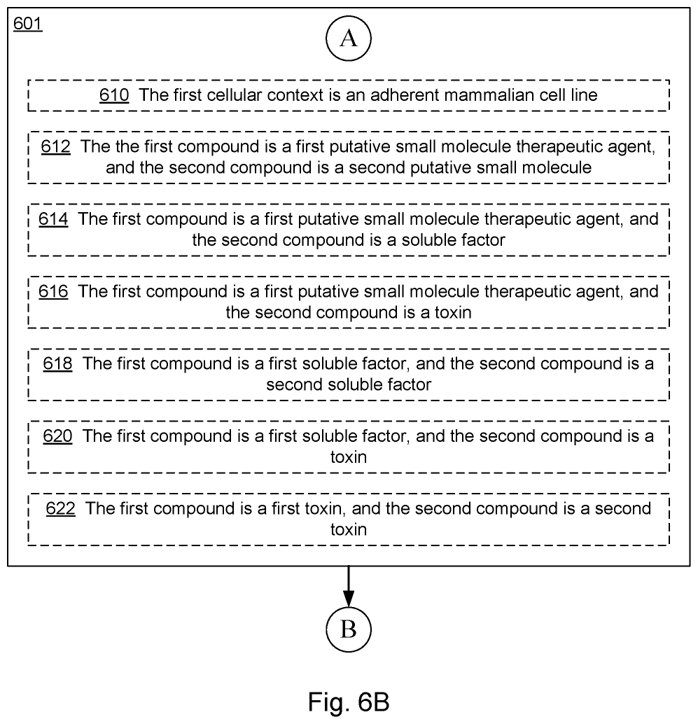

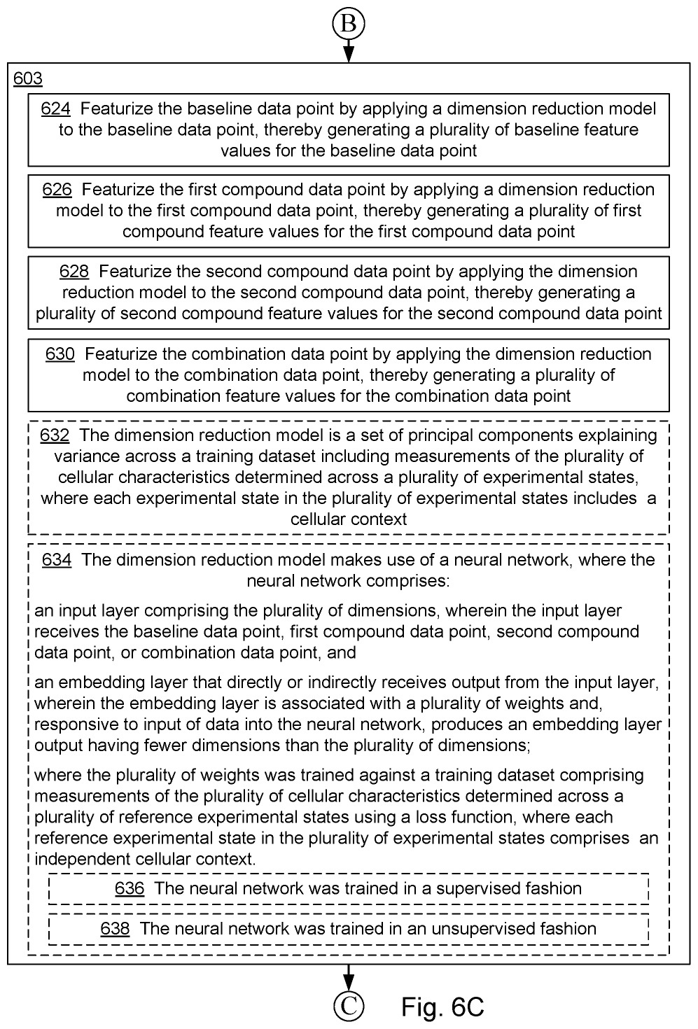

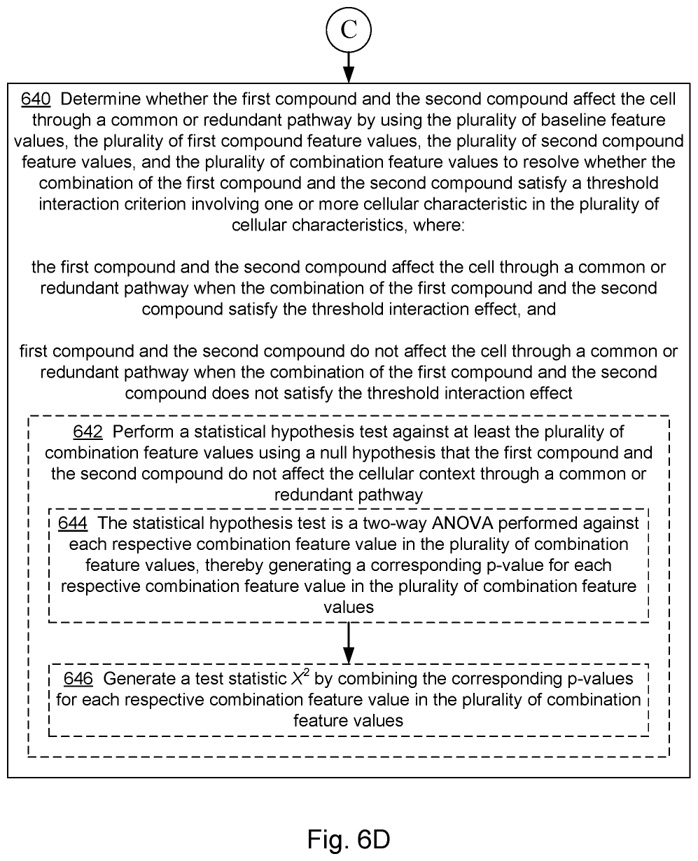

In another aspect, the present disclosure provides a method 600 for determining whether two compounds affect a cell through a common or redundant pathway, in a cell based assay. Method 600 begins with a block 601 which is illustrated in FIGS. 6A and 6B. In some embodiments, the two compounds are independently selected from a putative drug candidate, a soluble factor, and a toxin, e.g., interactions between any combination of two compounds can be detected using method 600. The cell based assay is performed in a plurality of wells across one or more multiwell plates. For example, referring to the hypothetical example described above with reference to FIGS. 3A-3D, different instances of experimental states (e.g., a baseline state 104, first compound state 106, second compound state 108, and combination state 110) are established in different wells 354 of multiwell plate 352.

In some embodiments, as illustrated with reference to FIG. 1A, the methods described herein include establishing a plurality of instances of cell-based experimental conditions representative of experimental states, e.g., in the wells of one or more multiwell plate, and measuring cellular characteristics from each of the instances, thereby generating a raw data set 221 for the assay (e.g., containing characteristic measurements 113, 117-1, 117-2, and 119 for one or more corresponding baseline experimental states 222, first compound experimental states 226-1, second compound experimental states 226-2, and combination experimental states 228, respectively). In some embodiments, the measurements and/or combinations of measurements of cellular characteristics are generated from one or more images of wells corresponding to an experimental state (e.g., wells corresponding to a baseline state 104, a first compound state 106, a second compound state 108, and a combination state 110), e.g., using an image analysis package, such as CellProfiler™ (Ljosa and Carpenter, PLoS Comput Biol., 5(12):e1000603 (2009) which is hereby incorporated by reference herein). In some embodiments, each feature is derived from a combination of measurable characteristics selected from a color, a texture, and a size of the cell context, or an enumerated portion of the cell context.

The raw data (e.g., the characteristic measurements of each instance of an experimental state) contained in data set 221 is then used to generate a set of data points 231 for each corresponding experimental state (e.g., data points 133 for a baseline experimental state 232, 137-1 for a first compound experimental state 236-1, 137-2 for a second compound experimental state 236-1, and 139 for an combination experimental state 238). In other embodiments, the methods described herein begin with the processing of raw data sets 221 or data point sets 231. That is, in some embodiments, data obtained from cell-based assays, performed as described herein, is received by system 200, and the methods described herein use that data to identify the action of two compounds through a common or partially-redundant pathway, e.g., with respect to method 600.

Method 600 includes obtaining (602) a baseline data point for a baseline state (e.g., baseline data point 133, as illustrated in FIGS. 1A, 2C, and 3D, obtained from measurements of cellular characteristics in instances of experimental conditions representative of a baseline state 104). The baseline data point includes a plurality of dimensions, where each respective dimension in the plurality of dimensions of the baseline data point represents a corresponding measure of central tendency of a different cellular characteristic, e.g., in a plurality of cellular characteristics, determined across a plurality of baseline aliquots of cells representing the baseline state in corresponding wells, in the plurality of wells, where the baseline state includes a first cellular context.

For instance, each baseline cellular characteristic 113 is measured in each instance of an experimental condition representative of baseline state 104 in the assay (e.g., measurements 113-1-1 through 113-1-16 of the same characteristic are obtained from wells 354-1-1 through 354-1-16, respectively, in FIG. 3B) and then a measure of central tendency is determined across each of the measurements (e.g., the mean of measurements 113-1-1 through 113-1-16 of the first characteristic

$$( {\sum }_{i = 16}^{1} ( \frac{113 - 1 - 1 - 1 - i}{16} ) ) ,$$

as illustrated in FIG. 3D). The measures of central tendency for each of the characteristics representative of the experimental state are then concatenated into a baseline data point (e.g., data point 133) for the respective cellular context. In some embodiments, the measure of central tendency is an arithmetic mean, weighted mean, midrange, midhinge, trimean, geometric mean, geometric median, Winsorized mean, median, or mode of the cellular characteristic.

In some embodiments, each of the cellular characteristics is an optically-measureable characteristic. In other embodiments, at least one cellular characteristic in the plurality of cellular characteristics is measured using a non-optical measurement. Non-limiting examples of optically-measureable and non-optically measureable cellular characteristic are described herein, e.g., in the Cellular Characteristic sections provided below.

In some embodiments, each of the baseline aliquots of cells include a single cell type, e.g., a single type of mammalian cell. In some embodiments, each of the baseline aliquots of cells includes the same mixture of different cell types, e.g., a co-culture of two different mammalian cells. In yet other embodiments, the plurality of baseline aliquots of cells includes different cellular contexts in different instances of the experimental condition that is representative of the baseline state. As described in further detail below, in some embodiments, measurements of the same cellular characteristic are acquired from different cell types in order to account for changes in the characteristic, e.g., upon perturbation of gene expression, exposure to a compound, and/or both, that are specific to one cell type. For instance, referring back to the hypothetical experiment described above with reference to FIGS. 3B-3D, in some embodiments, each baseline experimental condition in wells 354-1-1 to 354-1-16 contains a different cell type, or every two wells across the first row contain a different cell type, or every three wells across the first row contain a different cell type, etc.

In one embodiment, the first cellular context is a mammalian cell line. In one embodiment, the first cellular context is an adherent mammalian cell line (610). In some embodiments, the first cellular context is a human cell. In some embodiments, the first cellular context is a primary human cell. Non-limiting examples of cell types useful for the methods provided herein are described herein, e.g., in the Cell Contexts section provided below.

Method 600 also includes obtaining (604) a first compound data point for a first compound state (e.g., first compound data point 137-1, as illustrated in FIGS. 1A, 2C, and 3D, obtained from measurements of cellular characteristics in instances of experimental conditions representative of a first compound state 108-1). The first compound data point includes the plurality of dimensions (e.g., the same number of dimensions as the corresponding baseline data point 133), each respective dimension in the plurality of dimensions of the first compound data point representing the measurement of central tendency of a different cellular characteristic, in the plurality of cellular characteristics (the same cellular characteristics that were measured for the corresponding baseline state), determined across a plurality of first compound aliquots of cells representing the first compound state (e.g., referring to a modification of the hypothetical example with reference to FIG. 3B, the cellular characteristics are measured for each well 354-2 across the second row of multiwell plate 352, each of which contains an instance of an experimental condition representative of the first compound state).

The first compound state includes a first perturbation of the first cellular context in which the first cellular context is exposed to a first compound. That is, the cellular context(s) used in the compound experimental conditions is the same as the cellular context used in the background experimental conditions. However, the cellular context is exposed to a first test compound, e.g., a candidate drug, a soluble factor, or a toxin. More details with respect to compounds useful for method 400 are described herein, e.g., in Compound Perturbation section provided below.

Method 600 also includes obtaining (606) a second compound data point for a second compound state (e.g., second compound data point 137-2, as illustrated in FIGS. 1A, 2C, and 3D, obtained from measurements of cellular characteristics in instances of experimental conditions representative of a second compound state 108-2). The second compound data point includes the plurality of dimensions (e.g., the same number of dimensions as the corresponding baseline data point 133 and first compound data point 137-1), each respective dimension in the plurality of dimensions of the second compound data point representing the measurement of central tendency of a different cellular characteristic, in the plurality of cellular characteristics (the same cellular characteristics that were measured for the corresponding baseline state and first compound state), determined across a plurality of second compound aliquots of cells representing the second compound state (e.g., referring to a modification of the hypothetical example with reference to FIG. 3B, the cellular characteristics are measured for each well 354-3 across the third row of multiwell plate 352, each of which contains an instance of an experimental condition representative of the second compound state).

The second compound state includes a second perturbation of the first cellular context in which the first cellular context is exposed to a second compound. That is, the cellular context(s) used in the second compound experimental conditions is the same as the cellular context used in the background experimental conditions and first compound experimental conditions. However, the cellular context is exposed to a second test compound, e.g., a candidate drug, a soluble factor, or a toxin.

Method 600 also includes obtaining (608) a combination data point for a combination state (e.g., combination data point 139, as illustrated in FIGS. 1A, 2C, and 3D, obtained from measurements of cellular characteristics in instances of experimental conditions representative of a combination state 110). The combination data point includes the plurality of dimensions (e.g., the same number of dimensions as the corresponding baseline data point 133, first compound data point 137-1, and second compound data point 137-2), each respective dimension in the plurality of dimensions of the combination data point representing the measurement of central tendency of a different cellular characteristic, in the plurality of cellular characteristics (the same cellular characteristics that were measured for the corresponding baseline state, first compound state, and second compound state), determined across a corresponding plurality of combination aliquots of cells representing the combination state (e.g., referring to the modified hypothetical example with reference to FIG. 3B, the cellular characteristics are measured for each well 354-4 across the fourth row of multiwell plate 352, each of which contains an instance of an experimental condition representative of the combination state).

The combination state includes a third perturbation of the first cellular context in which the first cellular context is exposed to the first compound (the same compound that was used in the first compound state) and the second compound (the same compound that was used in the second compound state). Similarly, as described herein with reference to compound states, the concentration of the first or second test compound may be selected based on various known or expected properties of the compound. However, it is desirable that the concentration of the first and second test compound in the combination state be the same as the concentration of the first and second test compound used in the first and second compound states, such that any difference in the measured cellular characteristics, relative to the first or second compound state, attributable to the interaction between the first and second compounds, can more easily be identified.

In some embodiments, the first compound is a first putative small molecule therapeutic agent (e.g., a compound that is not a polypeptide, a polynucleotide, or a signaling molecule endogenous to the first cellular context), and the second compound is a second putative small molecule (612). In some embodiments, the first compound is a putative small molecule therapeutic agent, and the second compound is a soluble factor (e.g., a signaling molecule endogenous to the first cell context) (614). In some embodiments, the first compound is a putative small molecule therapeutic agent, the second compound is a toxin (616). In some embodiments, the first compound is a first soluble factor, and the second compound is a second soluble factor (618). In some embodiments, the first compound is a soluble factor, and the second compound is a toxin (620). In some embodiments, the first compound is a first toxin, and the second compound is a second toxin (622).

Method 600 proceeds to a block 603 illustrated in FIG. 6C. Method 600 then includes featurizing the data points obtained above (e.g., baseline data point 133, first compound data point 137-1, second compound data point 137-2, and combination data point 139), to reduce the dimensionality of the data and improve upon any scarcity in the data, e.g., as described above with reference to step 140 in FIG. 1A. As described herein, the lower dimensionality of the featurized data point decreases the processing time required to analyze the data, thereby creating a more efficient processing system 200. Additionally, in some embodiments, featurizing the data reduces or removes multicollinearity between cellular characteristics, which are intercorrelations or inter-associations among independent variables in a data set. Multicollinerarity can cause problems during data analysis that result in models with high errors, e.g., due to poorly estimated partial regression coefficients during regression analysis. Accordingly, featurizing the data can both improve the speed of the process, as well as the results.

Method 600 includes featurizing (624) the baseline data point (e.g., baseline data point 133) by applying a dimension reduction model to the baseline data point, thereby generating a plurality of baseline feature values for the baseline data point. The plurality of baseline feature values define a baseline featurized vector (e.g., baseline feature values F_{B1} through F_{Bn} of baseline featurized data point 143) that has fewer dimensions than the corresponding data point (e.g., baseline data point 133).

Method 600 includes featurizing (626) the first compound data point (e.g., first compound data point 137-1) by applying the dimension reduction model (the same model as used to featurize baseline data point 133) to the first compound data point, thereby generating a plurality of first compound feature values for the first compound data point. The plurality of first compound feature values define a first compound featurized vector (e.g., first compound feature values F_{D1-1} through F_{Dn-1} of first compound featurized data point 147-1) that has fewer dimensions than the corresponding data point (e.g., first compound data point 137-1).

Method 600 includes featurizing (628) the second compound data point (e.g., second compound data point 137-2) by applying the dimension reduction model (the same model as used to featurize baseline data point 133 and first compound data point 137-1) to the second compound data point, thereby generating a plurality of second compound feature values for the second compound data point. The plurality of second compound feature values define a second compound featurized vector (e.g., second compound feature values F_{D1-2} through F_{Dn-2} of second compound featurized data point 147-2) that has fewer dimensions than the corresponding data point (e.g., second compound data point 137-2).

Method 600 includes featurizing (630) the combination data point (e.g., combination data point 139) by applying the dimension reduction model (the same model as used to featurize baseline data point 133, first compound data point 137-1, and second compound data point 137-2) to the combination data point, thereby generating a plurality of combination feature values for the combination data point. The plurality of combination feature values define a combination featurized vector (e.g., combination feature values F_{C1} through F_{Cn} of combination featurized data point 149) that has fewer dimensions than the corresponding data point (e.g., combination data point 139).

Featurization of the cellular characteristics is performed by a dimensional reduction technique that uses a statistical feature selection or feature extraction procedure known in the art, for example, principal component analysis, non-negative matrix factorization, kernel PCA, graph-based kernel PCA, linear discriminant analysis, generalized discriminant analysis, and use of an autoencoder. This, in turn, reduces the computational burden of analyzing the data set by compressing the data in order to make the method more computationally efficient, e.g., by allowing the computer to apply an algorithm to the smaller dataset rather than the full dataset. Further information about featurization is described herein, e.g., in the Dimension Reduction section provided below.

In some embodiments, the dimension reduction model is a set of principal components (632) explaining variance across a training dataset including measurements of the plurality of cellular characteristics determined across a plurality of experimental states, where each experimental state in the plurality of experimental states includes a cellular context. For instance, in some embodiments, a set of principal components is learned against a training data set that includes measurements of the same cellular characteristics as used in method 600. In some embodiments, the training data set includes data derived from measurements of cellular characteristics from a plurality of instances of baseline states, e.g., unperturbed cell contexts. In some embodiments, the training data set includes data derived from measurements of cellular characteristics from a plurality of instances of perturbation states, e.g., cell contexts in which the expression of one or more target genes is perturbed, relative to a corresponding unperturbed baseline cellular context. In some embodiments, the training data set includes data derived from measurements of cellular characteristics from a plurality of instances of compound states, e.g., cell contexts which are exposed to one or more test compounds (e.g., candidate drugs, soluble factors, and/or toxins). In some embodiments, the training data set includes data derived from measurements of cellular characteristics from a plurality of instances of combination states, e.g., cell contexts in which the expression of one or more target genes is perturbed, relative to a corresponding unperturbed baseline cellular context, and is exposed to one or more test compounds (e.g., candidate drugs, soluble factors, and/or toxins). In some embodiments, the training data set includes data derived from measurements of cellular characteristics from a plurality of instances of baseline states and a plurality of instances of perturbation states and/or compound states. In some embodiments, the training data set includes data derived from measurements of cellular characteristics from a plurality of instances of baseline states, a plurality of instances of perturbation states, a plurality of instances of compound states, and a plurality of instances of combination states.

In some embodiments, the dimension reduction model makes use of a neural network (634), (e.g., as illustrated in FIG. 9) where the neural network includes: (i) an input layer comprising the plurality of dimensions (e.g., input layer 902), where the input layer receives the baseline data point (e.g., baseline data point 133), first compound data point (e.g., first compound data point 137-1), second compound data point (e.g., second compound data point 137-2), or combination data point (e.g., combination data point 139), and (ii) an embedding layer (e.g., embedding layer 910) that directly or indirectly receives output from the input layer. The embedding layer is associated with a plurality of weights (e.g., applied via connections 908) and, responsive to input of data into the neural network, produces an embedding layer (e.g., embedding layer 910) output having fewer dimensions than the plurality of dimensions (e.g., input layer 902, illustrated in FIG. 9, has m-dimensions, while embedding layer 910 has n-dimensions, where m>n). The plurality of weights (e.g., used in neural network 900) was trained against a training dataset including measurements of the plurality of cellular characteristics determined across a plurality of reference experimental states using a loss function, where each reference experimental state in the plurality of experimental states comprises an independent cellular context. For instance, in some embodiments, a neural network (e.g., neural network 900) is trained against a training data set that includes measurements of the same cellular characteristics as used in method 600.

In some embodiments, the training data set includes data derived from measurements of cellular characteristics from a plurality of instances of baseline states, e.g., unperturbed cell contexts. In some embodiments, the training data set includes data derived from measurements of cellular characteristics from a plurality of instances of perturbation states, e.g., cell contexts in which the expression of one or more target genes is perturbed, relative to a corresponding unperturbed baseline cellular context. In some embodiments, the training data set includes data derived from measurements of cellular characteristics from a plurality of instances of compound states, e.g., cell contexts which are exposed to one or more test compounds (e.g., candidate drugs, soluble factors, and/or toxins). In some embodiments, the training data set includes data derived from measurements of cellular characteristics from a plurality of instances of combination states, e.g., cell contexts in which the expression of one or more target genes is perturbed, relative to a corresponding unperturbed baseline cellular context, and is exposed to one or more test compounds (e.g., candidate drugs, soluble factors, and/or toxins). In some embodiments, the training data set includes data derived from measurements of cellular characteristics from a plurality of instances of baseline states and a plurality of instances of perturbation states and/or compound states. In some embodiments, the training data set includes data derived from measurements of cellular characteristics from a plurality of instances of baseline states, a plurality of instances of perturbation states, a plurality of instances of compound states, and a plurality of instances of combination states.

In some embodiments, e.g., referring to FIG. 9, neural network 900 includes an input layer 902 including a plurality of dimensions (e.g., the same number of dimensions as data points 133, 137-1, 137-2, and 139, where the input layer receives the baseline data point, perturbation data point, compound data point, or combination data point. For example, as illustrated in FIG. 9, each dimension of input layer 902 receives a term Cᵢ of combination data point 139 (e.g., as illustrated in FIG. 1A). Neural network 900 also includes embedding layer 910 linked to input layer 902 directly or indirectly. For instance, in some embodiments, neural network 900 includes one or more hidden layers 906 positioned between input layer 902 and embedding layer 910 (e.g., coupled via connections 904 and 908). In other embodiments, there are no hidden layers between input layer 902 and embedding layer 910, such that embedding layer 910 receives the output of input layer 902 directly. Embedding layer 910 includes fewer dimensions (e.g., n dimensions) than input layer 902 (e.g., m dimensions, where m>n). Neural network 900 also includes output layer 918 that directly or indirectly receives the output of embedding layer 910. For instance, in some embodiments, neural network 900 includes one or more hidden layers 914 positioned between embedding layer 910 and output layer 918 (e.g., coupled via connections 912 and 916). In other embodiments, there are no hidden layers between embedding layer 910 and output layer 918, such that output layer receives the output of embedding layer 910 directly (e.g., via connections 916). In some embodiments, output layer 918 predicts an attribute of a test experimental state that includes a cellular context, responsive to loading a test data point that includes the plurality of dimensions and is representative of the test state (e.g., a data point of measures of central tendency of cellular characteristics measured across a plurality of instances of the test state). In some embodiments, the neural network is the neural network was trained against a training dataset comprising measurements of the plurality of cellular characteristics determined across a plurality of reference experimental states, wherein each reference experimental state in the plurality of experimental states comprises an independent cellular context, e.g., as described above. The portion of the neural network that serves as the dimension reduction model consists of the input layer (e.g., input layer 902), the embedding layer (e.g., embedding layer 910), and all hidden layers (e.g., optional hidden layer 906) linking the input layer to the embedding layer, such that the output of the embedding layer are the baseline feature values, perturbation feature values, compound feature values, or combination feature values (e.g., as illustrated in FIG. 9, each dimension of input layer 902 receives a term Cᵢ of combination data point 139 and each layer of embedding layer 910 outputs a term F_{ci} of combination state featurized vector 149).

Methods for training neural networks are generally known in the art, e.g., using back propagation techniques, such as stochastic gradient descent. Accordingly, in some embodiments, neural network is trained in a supervised fashion (636). In other embodiments, e.g., where the neural network is an autoencoder, the neural network is trained in an unsupervised fashion (638). For more information regarding artificial neural networks, see, for example, Abiodun O I, et al., Heliyon, 4(11):e00938 (2018), the content of which is incorporated herein by reference.

With reference to FIG. 6D, method 600 then includes determining (640) whether the first compound (the compound included in first compound state 108-1) and the second compound (the compound included in second compound state 108-2) affect the cell through a common or redundant pathway by using the plurality of baseline feature values (e.g., baseline featurized data point 143), the plurality of first compound feature values (e.g., first compound featurized data point 147-1), the plurality of second compound feature values (e.g., second compound featurized data point 147-2), and the plurality of combination feature values (e.g., combination featurized data point 149) to resolve whether the combination of the first compound and the second compound satisfy a threshold interaction criterion involving one or more cellular characteristic in the plurality of cellular characteristics (e.g., whether the change in cellular characteristics in the combination state, relative to the cellular characteristics in the baseline state, is significantly more or less than would be expected from the combination of changes, relative to the baseline state, observed in the first compound state and the second compound state). The first compound and the second compound affect the cell through a common or redundant pathway when the combination of the first compound and the second compound satisfy the threshold interaction effect, whereas the first compound and the second compound do not affect the cell through a common or redundant pathway when the combination of the first compound and the second compound does not satisfy the threshold interaction effect.

In some embodiments, a statistical hypothesis test, using the feature values derived from the cell assay data, is performed (642) to determine whether the first compound and the second compound affect the cell through a common or redundant pathway. In some embodiments, the statistical hypothesis test is performed (640) against at least the plurality of combination feature values using a null hypothesis that the compound does not interact with the gene. In some embodiments, the statistical hypothesis test is a two-way ANOVA performed (644) against each respective combination feature value in the plurality of combination feature values, thereby generating a corresponding p-value for each respective combination feature value in the plurality of combination feature values. For instance, in some embodiments, a two-way ANOVA is performed against each feature F_{ci} of combination featurized data set 149, using corresponding features F_{Bi} of baseline featurized data set 143, F_{D1-1} of first compound featurized data set 147-1, and F_{Di-2} of second compound featurized data set 147-2, thereby generating a corresponding p-value 159 for each feature F_{ci} of combination featurized data set 149.

In some embodiments, determining whether the first compound and the second compound affect the cell through a common or redundant pathway includes generating (646) a test statistic X² by combining the corresponding p-values (e.g., p-values 159) for each respective combination feature value (e.g., F_{ci}) in the plurality of combination feature values (e.g., featurized data set 149). Methods of meta-analysis combining p-values are known in the art and include, for example, Fischer's method, Pearson's method, George's method, Edgington's method, Stouffer's method, Tippett's method, use of a Beta distribution, use of a truncated gamma distribution, and use of other general distribution functions. For more information regarding methods for combining p-values, and rationales for choosing a particular meta-analysis method, see, for example, Heard N A, “Choosing Between Methods of Combining p-values,” arXiv:1707.06897v4 [stat.ME] 14 Dec. 2017, the content of which is hereby incorporated by reference.

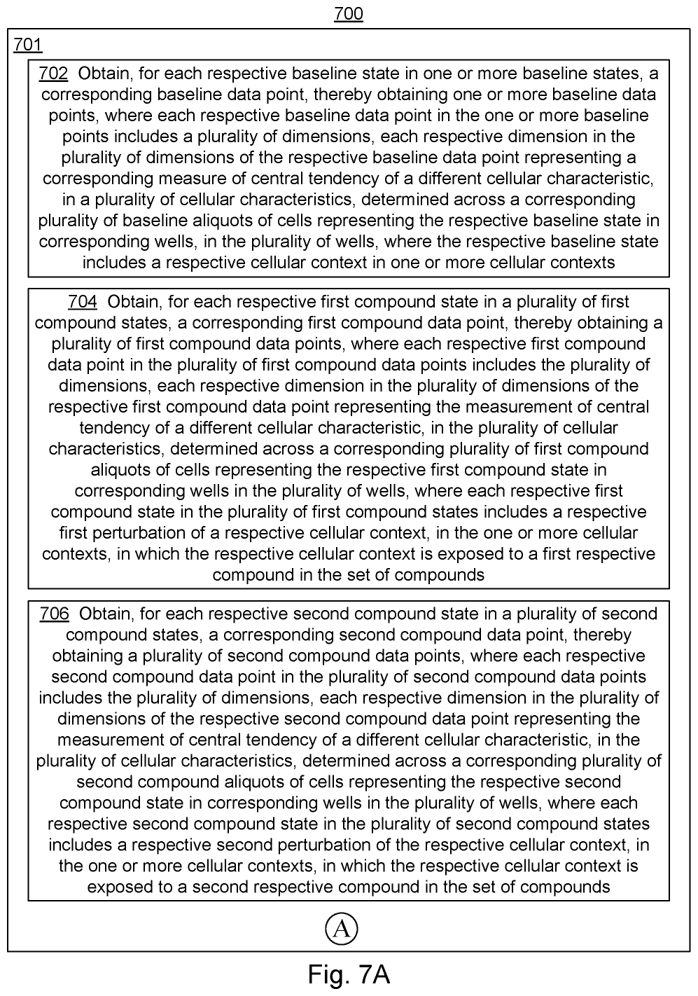

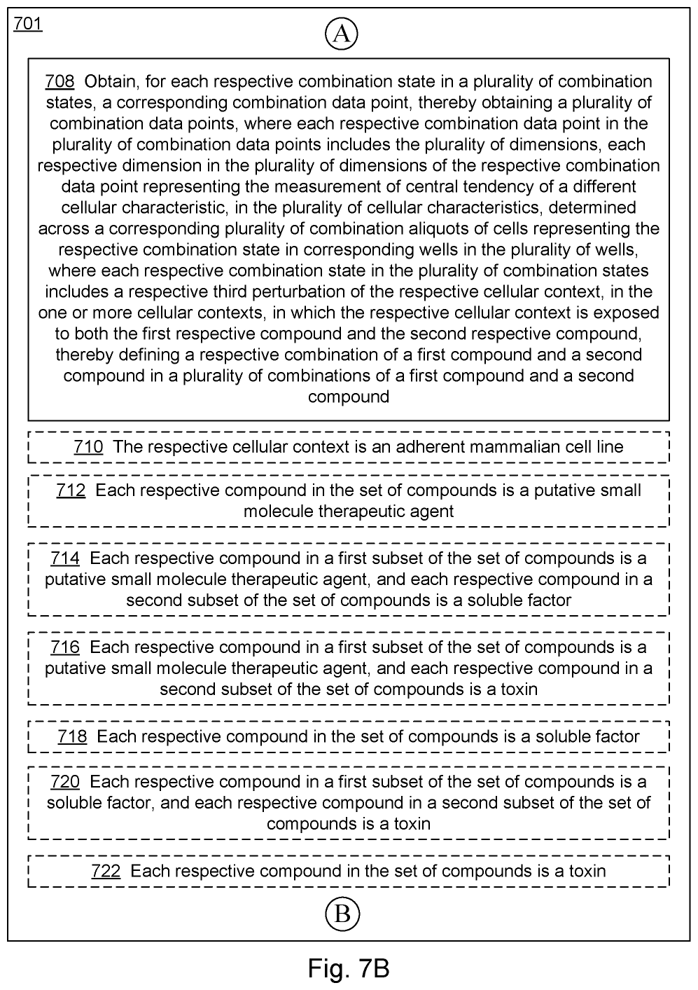

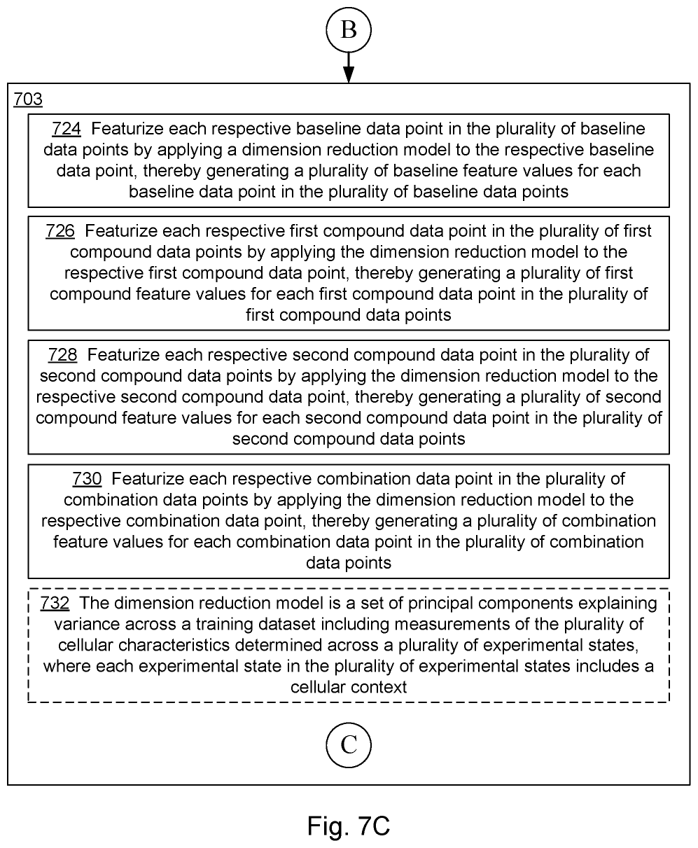

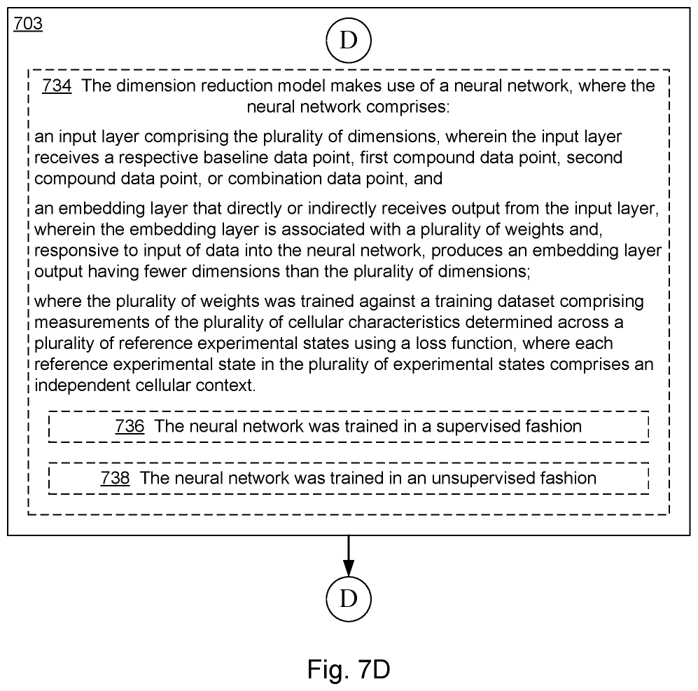

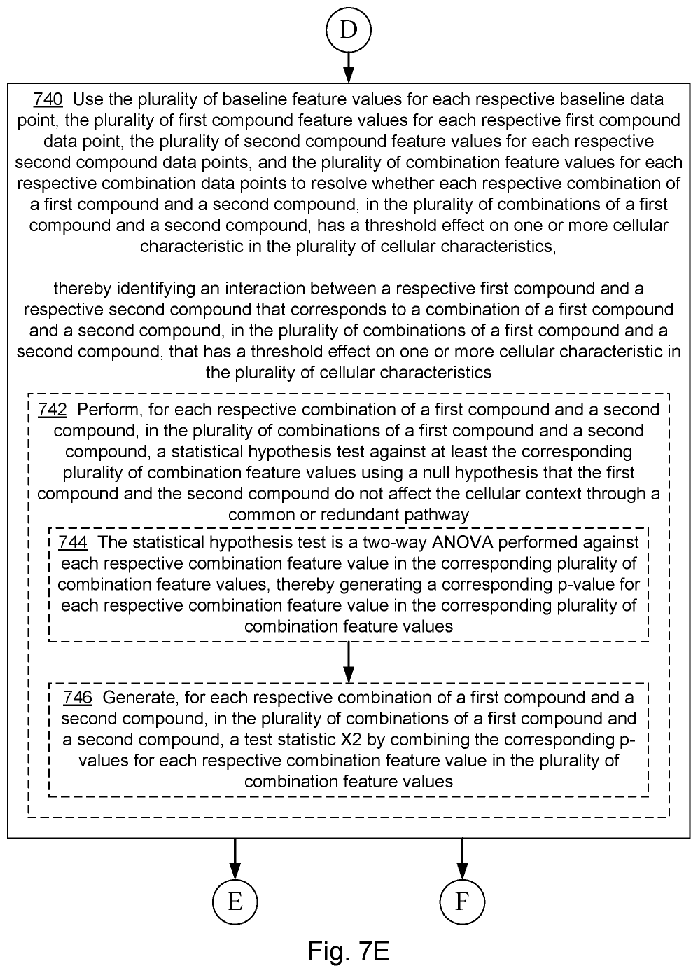

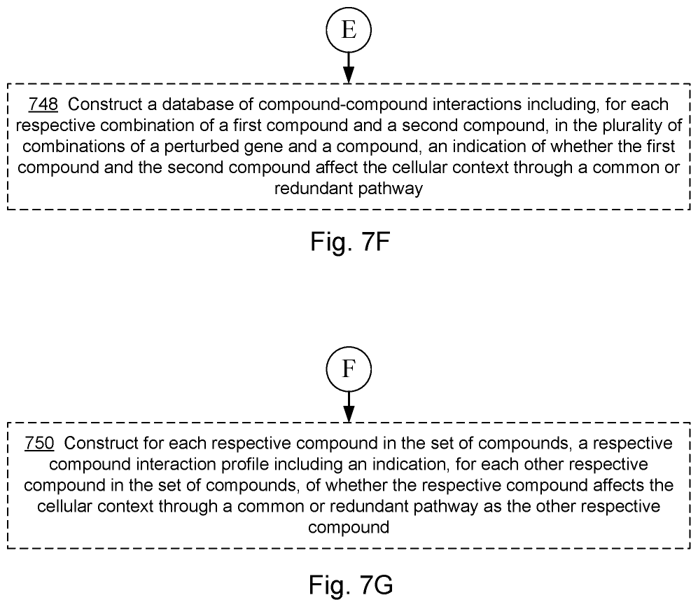

In a related aspect, the disclosure also provides a method 700 for identifying interactions between two perturbations in a plurality of perturbations, e.g., in an interaction screen performed a plurality of perturbation states. Method 700 begins with a block 701 which is illustrated in FIGS. 7A and 7B. As described for method 600 above, and illustrated in FIG. 7, method 700 includes analyzing pairwise interactions between respective perturbations, e.g., gene expression perturbation and/or exposure to a target compound, e.g., a candidate drug, soluble factor, or toxin. In some embodiments, method 700 is performed with at least 10 different perturbation, resulting in analysis of 45 pairwise interactions. In some embodiments, method 700 is performed with at least 25 different perturbations, or at least 50, 100, 200, 300, 400, 500, 600, 700, 800, 900, 1000, or more different perturbations.

In some embodiments, as illustrated with reference to FIG. 1A, the methods described herein include establishing a plurality of instances of cell-based experimental conditions representative of experimental states, e.g., in the wells of one or more multiwell plate, and measuring cellular characteristics from each of the instances, thereby generating a raw data set 221 for the assay (e.g., containing characteristic measurements 113, 117-1, 117-2, and 119 for one or more corresponding baseline experimental states 222, first compound experimental states 226-1, second compound experimental states 226-2, and combination experimental states 228, respectively). In some embodiments, the measurements and/or combinations of measurements of cellular characteristics are generated from one or more images of wells corresponding to an experimental state (e.g., wells corresponding to a baseline state 104, a first compound state 106, a second compound state 108, and a combination state 110), e.g., using an image analysis package, such as CellProfiler™ (Ljosa and Carpenter, PLoS Comput Biol., 5(12):e1000603 (2009) which is hereby incorporated by reference herein). In some embodiments, each feature is derived from a combination of measurable characteristics selected from a color, a texture, and a size of the cell context, or an enumerated portion of the cell context.

The raw data (e.g., the characteristic measurements of each instance of an experimental state) contained in data set 221 is then used to generate a set of data points 231 for each corresponding experimental state (e.g., data points 133 for a baseline experimental state 232, 137-1 for a first compound experimental state 236-1, 137-2 for a second compound experimental state 236-1, and 139 for an combination experimental state 238). In other embodiments, the methods described herein begin with the processing of raw data sets 221 or data point sets 231. That is, in some embodiments, data obtained from cell-based assays, performed as described herein, is received by system 200, and the methods described herein use that data to identify the action of two compounds through a common or partially-redundant pathway, e.g., with respect to method 700.

Method 700 includes obtaining (702) for each respective baseline state in one or more baseline states, a corresponding baseline data point (e.g., baseline data point 133 thereby obtaining one or more baseline data points, where each respective baseline data point in the one or more baseline points includes a plurality of dimensions, as illustrated in FIGS. 1A, 2C, and 3D, obtained from measurements of cellular characteristics in instances of experimental conditions representative of a baseline state 104), each respective dimension in the plurality of dimensions of the respective baseline data point representing a corresponding measure of central tendency of a different cellular characteristic, in a plurality of cellular characteristics, determined across a corresponding plurality of baseline aliquots of cells representing the respective baseline state in corresponding wells, in the plurality of wells, where the respective baseline state includes a respective cellular context in one or more cellular contexts

For instance, each baseline cellular characteristic 113 is measured in each instance of an experimental condition representative of baseline state 104 in the assay (e.g., measurements 113-1-1 through 113-1-16 of the same characteristic are obtained from wells 354-1-1 through 354-1-16, respectively, in FIG. 3B) and then a measure of central tendency is determined across each of the measurements (e.g., the mean of measurements 113-1-1 through 113-1-16 of the first characteristic (Σ_(i=16)\hat{ }1 [symbol] [symbol] ((113−1−1−1−i)/16) [symbol] ), as illustrated in FIG. 3D). The measures of central tendency for each of the characteristics representative of the experimental state are then concatenated into a baseline data point (e.g., data point 133) for the respective cellular context. In some embodiments, the measure of central tendency is an arithmetic mean, weighted mean, midrange, midhinge, trimean, geometric mean, geometric median, Winsorized mean, median, or mode of the cellular characteristic.

In some embodiments, each of the cellular characteristics is an optically-measureable characteristic. In other embodiments, at least one cellular characteristic in the plurality of cellular characteristics is measured using a non-optical measurement. Non-limiting examples of optically-measureable and non-optically measureable cellular characteristic are described herein, e.g., in the Cellular Characteristic sections provided below.

In some embodiments, each of the baseline aliquots of cells include a single cell type, e.g., a single type of mammalian cell. In some embodiments, each of the baseline aliquots of cells includes the same mixture of different cell types, e.g., a co-culture of two different mammalian cells. In yet other embodiments, the plurality of baseline aliquots of cells includes different cellular contexts in different instances of the experimental condition that is representative of the baseline state. As described in further detail below, in some embodiments, measurements of the same cellular characteristic are acquired from different cell types in order to account for changes in the characteristic, e.g., upon perturbation of gene expression, exposure to a compound, and/or both, that are specific to one cell type. For instance, referring back to the hypothetical experiment described above with reference to FIGS. 3B-3D, in some embodiments, each baseline experimental condition in wells 354-1-1 to 354-1-16 contains a different cell type, or every two wells across the first row contain a different cell type, or every three wells across the first row contain a different cell type, etc.

In one embodiment, the respective cellular context is a mammalian cell line. In one embodiment, the respective cellular context is an adherent mammalian cell line (710). In some embodiments, the respective cellular context is a human cell. In some embodiments, the respective cellular context is a primary human cell. Non-limiting examples of cell types useful for the methods provided herein are described herein, e.g., in the Cell Contexts section provided below.

Method 700 also includes obtaining (704) for each respective first compound in a plurality of first compound states, a corresponding first compound data point (e.g., first compound data point 137-1, as illustrated in FIGS. 1A, 2C, and 3D, obtained from measurements of cellular characteristics in instances of experimental conditions representative of a first compound state 108-1), thereby obtaining a plurality of first compound data points. Each respective first compound data point in the plurality of first compound data points includes the plurality of dimensions (e.g., the same number of dimensions as the corresponding baseline data point 133), each respective dimension in the plurality of dimensions of the respective first compound data point representing the measurement of central tendency of a different cellular characteristic, in the plurality of cellular characteristics, determined across a corresponding plurality of first compound aliquots of cells representing the respective first compound state in corresponding wells in the plurality of wells (e.g., referring to a modification of the hypothetical example with reference to FIG. 3B, the cellular characteristics are measured for each well 354-2 across the second row of multiwell plate 352, each of which contains an instance of an experimental condition representative of the first compound state). Each respective first compound state in the plurality of first compound states includes a respective first perturbation of a respective cellular context, in the one or more cellular contexts, in which the respective cellular context is exposed to a first respective compound in the set of compounds. That is, the cellular context(s) used in the compound experimental conditions is the same as the cellular context used in the background experimental conditions. However, the cellular context is exposed to a first test compound, e.g., a candidate drug, a soluble factor, or a toxin. More details with respect to compounds useful for method 400 are described herein, e.g., in Compound Perturbation section provided below.

Method 700 also includes obtaining (706) for each respective second compound state in a plurality of second compound states, a corresponding second compound data point (e.g., second compound data point 137-2, as illustrated in FIGS. 1A, 2C, and 3D, obtained from measurements of cellular characteristics in instances of experimental conditions representative of a second compound state 108-2), thereby obtaining a plurality of second compound data points. Each respective second compound data point in the plurality of second compound data points includes the plurality of dimensions (e.g., the same number of dimensions as the corresponding baseline data point 133 and first compound data point 137-1), each respective dimension in the plurality of dimensions of the respective second compound data point representing the measurement of central tendency of a different cellular characteristic (the same cellular characteristics that were measured for the corresponding baseline state and first compound state), in the plurality of cellular characteristics, determined across a corresponding plurality of second compound aliquots of cells representing the respective second compound state in corresponding wells in the plurality of wells (e.g., referring to a modification of the hypothetical example with reference to FIG. 3B, the cellular characteristics are measured for each well 354-3 across the third row of multiwell plate 352, each of which contains an instance of an experimental condition representative of the second compound state). Each respective second compound state in the plurality of second compound states includes a respective second perturbation of the respective cellular context, in the one or more cellular contexts, in which the respective cellular context is exposed to a second respective compound in the set of compounds. That is, the cellular context(s) used in the second compound experimental conditions is the same as the cellular context used in the background experimental conditions and first compound experimental conditions. However, the cellular context is exposed to a second test compound, e.g., a candidate drug, a soluble factor, or a toxin.

Method 700 also includes obtaining (708) for each respective combination state in a plurality of combination states, a corresponding combination data point (e.g., combination data point 139, as illustrated in FIGS. 1A, 2C, and 3D, obtained from measurements of cellular characteristics in instances of experimental conditions representative of a combination state 110), thereby obtaining a plurality of combination data points. Each respective combination data point in the plurality of combination data points includes the plurality of dimensions (e.g., the same number of dimensions as the corresponding baseline data point 133, first compound data point 137-1, and second compound data point 137-2), each respective dimension in the plurality of dimensions of the respective combination data point representing the measurement of central tendency of a different cellular characteristic, in the plurality of cellular characteristics (the same cellular characteristics that were measured for the corresponding baseline state, first compound state, and second compound state), determined across a corresponding plurality of combination aliquots of cells representing the respective combination state in corresponding wells in the plurality of wells (e.g., referring to the modified hypothetical example with reference to FIG. 3B, the cellular characteristics are measured for each well 354-4 across the fourth row of multiwell plate 352, each of which contains an instance of an experimental condition representative of the combination state).

Each respective combination state in the plurality of combination states includes a respective third perturbation of the respective cellular context, in the one or more cellular contexts, in which the respective cellular context is exposed to both the first respective compound (e.g., referring to the modified hypothetical example with reference to FIG. 3B, the cellular characteristics are measured for each well 354-4 across the fourth row of multiwell plate 352, each of which contains an instance of an experimental condition representative of the combination state) and the second respective compound (the same compound that was used in the second compound state), thereby defining a respective combination of a first compound and a second compound in a plurality of combinations of a first compound and a second compound. Similarly, as described herein with reference to compound states, the concentration of the first or second test compound may be selected based on various known or expected properties of the compound. However, it is desirable that the concentration of the first and second test compound in the combination state be the same as the concentration of the first and second test compound used in the first and second compound states, such that any difference in the measured cellular characteristics, relative to the first or second compound state, attributable to the interaction between the first and second compounds, can more easily be identified.

In some embodiments, each respective compound in the set of compounds is a putative small molecule therapeutic agent (e.g., a compound that is not a polypeptide, a polynucleotide, or a signaling molecule endogenous to the first cellular context), and the second compound is a second putative small molecule (712). In some embodiments, the respective compound in a first subset of the set of compounds is a putative small molecule therapeutic agent, and each respective compound in a second set of the compounds is a soluble factor (e.g., a signaling molecule endogenous to the first cell context) (714). In some embodiments, the respective compound in a first subset of the set of compounds is a putative small molecule therapeutic agent, each respective compound in a second subset of the set of compounds is a toxin (716). In some embodiments, each respective compound in the set of compounds is a soluble factor (718). In some embodiments, each respective compound in a first subset of the set of compounds is a soluble factor, and each respective compound in a second subset of the set of compounds is a toxin (720). In some embodiments, each respective compound in the set of compounds is a toxin (722).

Method 700 proceeds to a block 703 illustrated in FIGS. 7C and 7D. Method 700 then includes featurizing the data points obtained above (e.g., baseline data point 133, first compound data point 137-1, second compound data point 137-2, and combination data point 139), to reduce the dimensionality of the data and improve upon any scarcity in the data, e.g., as described above with reference to step 140 in FIG. 1A. As described herein, the lower dimensionality of the featurized data point decreases the processing time required to analyze the data, thereby creating a more efficient processing system 200. Additionally, in some embodiments, featurizing the data reduces or removes multicollinearity between cellular characteristics, which are intercorrelations or inter-associations among independent variables in a data set. Multicollinerarity can cause problems during data analysis that result in models with high errors, e.g., due to porrly estimated partial regression coefficients during regression analysis. Accordingly, featurizing the data can both improve the speed of the process, as well as the results.

Method 700 includes featurizing (724) each respective baseline data point (e.g., baseline data point 133) in the plurality of baseline data points by applying a dimension reduction model to the respective baseline data point, thereby generating a plurality of baseline feature values for each baseline data point in the plurality of baseline data points. The plurality of baseline feature values for a respective baseline data point define a baseline featurized vector (e.g., baseline feature values FB1 through FBn of baseline featurized data point 143) that has fewer dimensions than the corresponding data point (e.g., baseline data point 133).

Method 700 includes featurizing (726) each respective first compound data point (e.g., first compound data point 137-1) in the plurality of first compound data points by applying the dimension reduction model (the same model as used to featurize respective baseline data points) to the respective first compound data point, thereby generating a plurality of first compound feature values for each first compound data point in the plurality of first compound data points. The plurality of first compound feature values for a respective compound data point define a first compound featurized vector (e.g., first compound feature values FD1-1 through FDn-1 of first compound featurized data point 147-1) that has fewer dimensions than the corresponding data point (e.g., first compound data point 137-1).

Method 700 includes featurizing (728) each respective second compound data point (e.g., second compound data point 137-2) in the plurality of second compound data points by applying the dimension reduction model (the same model as used to featurize respective baseline data points and respective first compound data points) to the respective second compound data point, thereby generating a plurality of second compound feature values for each second compound data point in the plurality of second compound data points. The plurality of second compound feature values for a respective second compound data point define a second compound featurized vector (e.g., second compound feature values FD1-2 through FDn-2 of second compound featurized data point 147-2) that has fewer dimensions than the corresponding data point (e.g., second compound data point 137-2).

Method 700 includes featurizing (730) each respective combination data point (e.g., combination data point 139) in the plurality of combination data points by applying the dimension reduction model (the same model as used to featurize respective baseline data points, respective compound data points, and respective second compound data points) to the respective combination data point, thereby generating a plurality of combination feature values for each combination data point in the plurality of combination data points. The plurality of combination feature values for a respective combination data point define a combination featurized vector (e.g., combination feature values FC1 through FCn of combination featurized data point 149) that has fewer dimensions than the corresponding data point (e.g., combination data point 139).

Featurization of the cellular characteristics is performed by a dimensional reduction technique that uses a statistical feature selection or feature extraction procedure known in the art, for example, principal component analysis, non-negative matrix factorization, kernel PCA, graph-based kernel PCA, linear discriminant analysis, generalized discriminant analysis, and use of an autoencoder. This, in turn, reduces the computational burden of analyzing the data set by compressing the data in order to make the method more computationally efficient, e.g., by allowing the computer to apply an algorithm to the smaller dataset rather than the full dataset. Further information about featurization is described herein, e.g., in the Dimension Reduction section provided below.

In some embodiments, the dimension reduction model is a set of principal components (732) explaining variance across a training dataset including measurements of the plurality of cellular characteristics determined across a plurality of experimental states, where each experimental state in the plurality of experimental states includes a cellular context. For instance, in some embodiments, a set of principal components is learned against a training data set that includes measurements of the same cellular characteristics as used in method 700. In some embodiments, the training data set includes data derived from measurements of cellular characteristics from a plurality of instances of baseline states, e.g., unperturbed cell contexts. In some embodiments, the training data set includes data derived from measurements of cellular characteristics from a plurality of instances of perturbation states, e.g., cell contexts in which the expression of one or more target genes is perturbed, relative to a corresponding unperturbed baseline cellular context. In some embodiments, the training data set includes data derived from measurements of cellular characteristics from a plurality of instances of compound states, e.g., cell contexts which are exposed to one or more test compounds (e.g., candidate drugs, soluble factors, and/or toxins). In some embodiments, the training data set includes data derived from measurements of cellular characteristics from a plurality of instances of combination states, e.g., cell contexts in which the expression of one or more target genes is perturbed, relative to a corresponding unperturbed baseline cellular context, and is exposed to one or more test compounds (e.g., candidate drugs, soluble factors, and/or toxins). In some embodiments, the training data set includes data derived from measurements of cellular characteristics from a plurality of instances of baseline states and a plurality of instances of perturbation states and/or compound states. In some embodiments, the training data set includes data derived from measurements of cellular characteristics from a plurality of instances of baseline states, a plurality of instances of perturbation states, a plurality of instances of compound states, and a plurality of instances of combination states.

In some embodiments, the dimension reduction model makes use of a neural network (734), (e.g., as illustrated in FIG. 9) where the neural network includes: (i) an input layer comprising the plurality of dimensions (e.g., input layer 902), where the input layer receives the baseline data point (e.g., baseline data point 133), first compound data point (e.g., first compound data point 137-1), second compound data point (e.g., second compound data point 137-2), or combination data point (e.g., combination data point 139), and (ii) an embedding layer (e.g., embedding layer 910) that directly or indirectly receives output from the input layer. The embedding layer is associated with a plurality of weights (e.g., applied via connections 908) and, responsive to input of data into the neural network, produces an embedding layer (e.g., embedding layer 910) output having fewer dimensions than the plurality of dimensions (e.g., input layer 902, illustrated in FIG. 9, has m-dimensions, while embedding layer 910 has n-dimensions, where m>n). The plurality of weights (e.g., used in neural network 900) was trained against a training dataset including measurements of the plurality of cellular characteristics determined across a plurality of reference experimental states using a loss function, where each reference experimental state in the plurality of experimental states comprises an independent cellular context. For instance, in some embodiments, a neural network (e.g., neural network 900) is trained against a training data set that includes measurements of the same cellular characteristics as used in method 700.

In some embodiments, the training data set includes data derived from measurements of cellular characteristics from a plurality of instances of baseline states, e.g., unperturbed cell contexts. In some embodiments, the training data set includes data derived from measurements of cellular characteristics from a plurality of instances of perturbation states, e.g., cell contexts in which the expression of one or more target genes is perturbed, relative to a corresponding unperturbed baseline cellular context. In some embodiments, the training data set includes data derived from measurements of cellular characteristics from a plurality of instances of compound states, e.g., cell contexts which are exposed to one or more test compounds (e.g., candidate drugs, soluble factors, and/or toxins). In some embodiments, the training data set includes data derived from measurements of cellular characteristics from a plurality of instances of combination states, e.g., cell contexts in which the expression of one or more target genes is perturbed, relative to a corresponding unperturbed baseline cellular context, and is exposed to one or more test compounds (e.g., candidate drugs, soluble factors, and/or toxins). In some embodiments, the training data set includes data derived from measurements of cellular characteristics from a plurality of instances of baseline states and a plurality of instances of perturbation states and/or compound states. In some embodiments, the training data set includes data derived from measurements of cellular characteristics from a plurality of instances of baseline states, a plurality of instances of perturbation states, a plurality of instances of compound states, and a plurality of instances of combination states.

In some embodiments, e.g., referring to FIG. 9, neural network 900 includes an input layer 902 including a plurality of dimensions (e.g., the same number of dimensions as data points 133, 137-1, 137-2, and 139, where the input layer receives the baseline data point, perturbation data point, compound data point, or combination data point. For example, as illustrated in FIG. 9, each dimension of input layer 902 receives a term Cᵢ of combination data point 139 (e.g., as illustrated in FIG. 1A). Neural network 900 also includes embedding layer 910 linked to input layer 902 directly or indirectly. For instance, in some embodiments, neural network 900 includes one or more hidden layers 906 positioned between input layer 902 and embedding layer 910 (e.g., coupled via connections 904 and 908). In other embodiments, there are no hidden layers between input layer 902 and embedding layer 910, such that embedding layer 910 receives the output of input layer 902 directly. Embedding layer 910 includes fewer dimensions (e.g., n dimensions) than input layer 902 (e.g., m dimensions, where m>n). Neural network 900 also includes output layer 918 that directly or indirectly receives the output of embedding layer 910. For instance, in some embodiments, neural network 900 includes one or more hidden layers 914 positioned between embedding layer 910 and output layer 918 (e.g., coupled via connections 912 and 916). In other embodiments, there are no hidden layers between embedding layer 910 and output layer 918, such that output layer receives the output of embedding layer 910 directly (e.g., via connections 916). In some embodiments, output layer 918 predicts an attribute of a test experimental state that includes a cellular context, responsive to loading a test data point that includes the plurality of dimensions and is representative of the test state (e.g., a data point of measures of central tendency of cellular characteristics measured across a plurality of instances of the test state). In some embodiments, the neural network is the neural network was trained against a training dataset comprising measurements of the plurality of cellular characteristics determined across a plurality of reference experimental states, wherein each reference experimental state in the plurality of experimental states comprises an independent cellular context, e.g., as described above. The portion of the neural network that serves as the dimension reduction model consists of the input layer (e.g., input layer 902), the embedding layer (e.g., embedding layer 910), and all hidden layers (e.g., optional hidden layer 906) linking the input layer to the embedding layer, such that the output of the embedding layer are the baseline feature values, perturbation feature values, compound feature values, or combination feature values (e.g., as illustrated in FIG. 9, each dimension of input layer 902 receives a term Ci of combination data point 139 and each layer of embedding layer 910 outputs a term Fci of combination state featurized vector 149).

Methods for training neural networks are generally known in the art, e.g., using back propagation techniques, such as stochastic gradient descent. Accordingly, in some embodiments, neural network is trained in a supervised fashion (736). In other embodiments, e.g., where the neural network is an autoencoder, the neural network is trained in an unsupervised fashion (738). For more information regarding artificial neural networks, see, for example, Abiodun O I, et al., Heliyon, 4(11):e00938 (2018), the content of which is incorporated herein by reference.

With reference to FIG. 7E, method 700 then includes using (740) the plurality of baseline feature values (e.g., baseline featurized data point 143) for each respective baseline data point, the plurality of first compound feature values (e.g., first compound featurized data point 147-1) for each respective first compound data point, the plurality of second compound feature values (e.g., second compound featurized data point 147-2) for each respective second compound data points, and the plurality of combination feature values (e.g., combination featurized data point 149) for each respective combination data points to resolve whether each respective combination of a first compound and a second compound, in the plurality of combinations of a first compound and a second compound, has a threshold effect on one or more cellular characteristic (e.g., whether the change in cellular characteristics in the combination state, relative to the cellular characteristics in the baseline state, is significantly more or less than would be expected from the combination of changes, relative to the baseline state, observed in the first compound state and the second compound state) in the plurality of cellular characteristics, thereby identifying an interaction between a respective first compound and a respective second compound that corresponds to a combination of a first compound and a second compound, in the plurality of combinations of a first compound and a second compound, that has a threshold effect on one or more cellular characteristic in the plurality of cellular characteristics.

In some embodiments, for each respective combination of a first compound and a second compound, in the plurality of combinations of a first compound and a second compound, a statistical hypothesis test is performed (742) against at least the corresponding plurality of combination feature values using a null hypothesis that the first compound and the second compound do not affect the cellular context through a common or redundant pathway. In some embodiments, the statistical hypothesis test is a two-way ANOVA performed (744) against each respective combination feature value in the corresponding plurality of combination feature values, thereby generating a corresponding p-value for each respective combination feature value in the corresponding plurality of combination feature values.

In some embodiments, determining whether the first compound and the second compound affect the cell through a common or redundant pathway includes generating (746), for each respective combination of a first compound and a second compound, in the plurality of combinations of a first compound and a second compound, a test statistic X2 by combining the corresponding p-values for each respective combination feature value in the plurality of combination feature values. Methods of meta-analysis combining p-values are known in the art and include, for example, Fischer's method, Pearson's method, George's method, Edgington's method, Stouffer's method, Tippett's method, use of a Beta distribution, use of a truncated gamma distribution, and use of other general distribution functions. For more information regarding methods for combining p-values, and rationales for choosing a particular meta-analysis method, see, for example, Heard N A, “Choosing Between Methods of Combining p-values,” arXiv:1707.06897v4 [stat.ME] 14 Dec. 2017, the content of which is hereby incorporated by reference.

With reference to FIG. 7F, in some embodiments, the methods described herein further include constructing (748) a database of perturbation-perturbation interactions (e.g., compound-compound and/or compound-gene interactions). In one embodiment, this includes, for each respective combination of a first perturbation and a second perturbation, an indication of whether the first perturbation and the second perturbation affect the cellular context through a common or partially-redundant pathway. In one embodiment, this includes, an indication of whether the first compound and the second compound affect the cellular context through a common or redundant pathway.

The database of perturbation-perturbation interactions described above is used, in some embodiments, in a method for identifying an alternative therapy for a known treatment of a physiologic disorder. The method includes querying a database of perturbation-perturbation (e.g., compound-compound) interactions, constructed as described above, for a first compound that affects the cellular context through a common or partially-redundant pathway as a second compound, where the second compound is used in the known treatment of the physiologic disorder, thereby identifying the first compound for use in an alternative therapy for the physiologic disorder.

With reference to FIG. 7G, in some embodiments, the methods described herein further include constructing (750) a compound interaction profile for one or more compounds (to include each respective compound) tested as described above. The compound interaction profile includes an indication, for each other respective compound in the set of compounds, of whether the respective compound affects the cellular context through a common or redundant pathway as another respective compound.

The compound interaction profile described above is used, in some embodiments, in a method for identifying a mechanism of action for a test compound. The method includes comparing a compound interaction profile for the test compound to a plurality of annotated compound interaction profiles, where each respective annotated compound interaction profile in the plurality of annotated compound interaction profiles is for a corresponding compound, in a plurality of corresponding compounds, having a known mechanisms of action.

In one aspect, the disclosure provides a method 800 for determining whether a first cellular perturbation interacts with a second cellular perturbation in one of a specific cellular context and a background, in a cell based assay. In some embodiments, a compound used in the cell-based assay is a putative drug candidate, for example, a candidate therapeutic compound from a chemical library. In some embodiments, the compound is a soluble factor, e.g., a growth factor, chemokine, cytokine, adhesion molecule, protease, or shed receptor. In some embodiments, the compound is a toxin. The cell based assay is performed in a plurality of wells across one or more multiwell plates. For example, referring to the hypothetical example described above with reference to FIGS. 3A-3D, different instances of experimental states (e.g., baseline state 104, perturbation state 106, compound state 108, and combination state 110) are established in different wells 354 of multiwell plate 352.

In some embodiments, as illustrated with reference to FIG. 1A, the methods described herein include establishing a plurality of instances of cell-based experimental conditions representative of experimental states (e.g., baseline states 104, perturbation states 106, compound states 108, and/or combination states 110), e.g., in the wells of one or more multiwell plate, and measuring cellular characteristics from each of the instances, thereby generating a raw data set 221 for the assay (e.g., containing characteristic measurements 113, 115, 117, and/or 119 for one or more corresponding baseline experimental states 222, perturbation experimental states 224, compound experimental states 226, and/or combination experimental states 228). In some embodiments, the measurements and/or combinations of measurements of cellular characteristics are generated from one or more images of wells corresponding to an experimental state (e.g., wells corresponding to a baseline state 104, a perturbation state 106, a compound state 108, and/or a combination state 110), e.g., using an image analysis package, such as CellProfiler™ (Ljosa and Carpenter, PLoS Comput Biol., 5(12):e1000603 (2009) which is hereby incorporated by reference herein). In some embodiments, each feature is derived from a combination of measurable characteristics selected from a color, a texture, and a size of the cell context, or an enumerated portion of the cell context.

The raw data (e.g., the characteristic measurements of each instance of an experimental state) contained in data set 221 is then used to generate a set of data points 231 for each corresponding experimental state (e.g., data points 133 for a baseline experimental state 232, 135 for a perturbation experimental state 234, 137 for an experimental compound state 236, and/or 139 for an experimental combination state 238). In other embodiments, the methods described herein begin with the processing of raw data sets 221 or data point sets 231. That is, in some embodiments, data obtained from cell-based assays, performed as described herein, is received by system 200, and the methods described herein use that data to identify interactions between various biological agents, e.g., with respect to method 800, interactions between a gene and a compound.

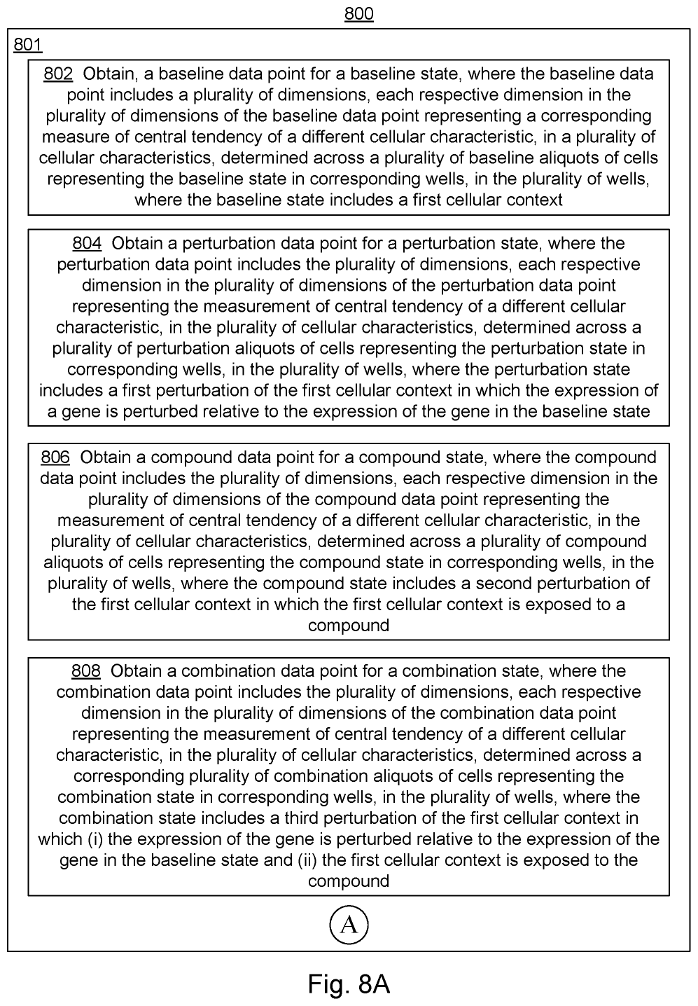

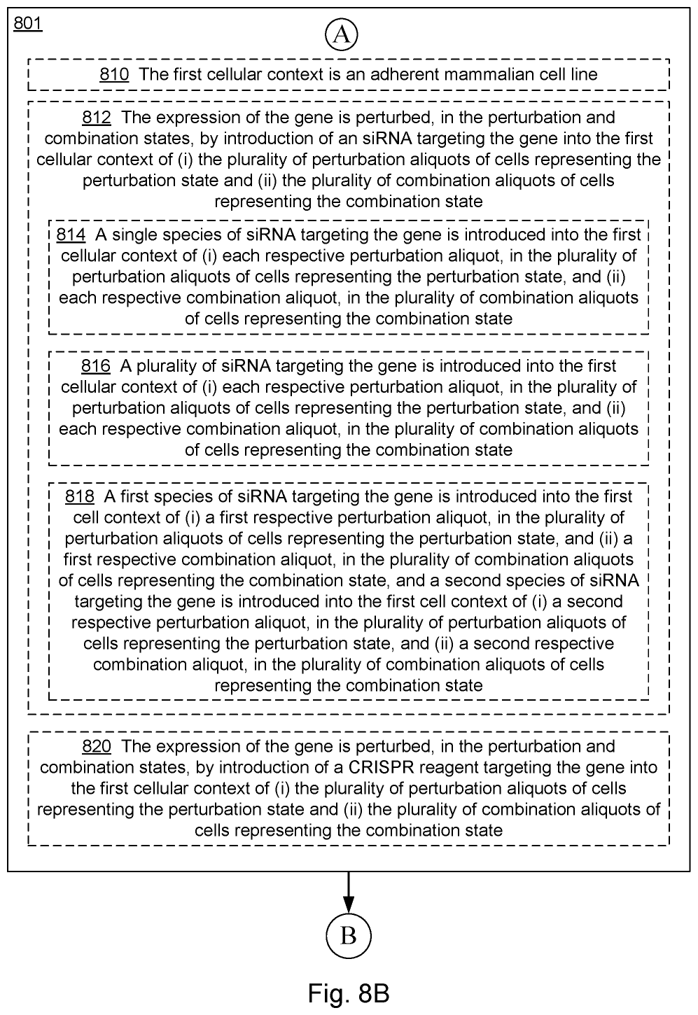

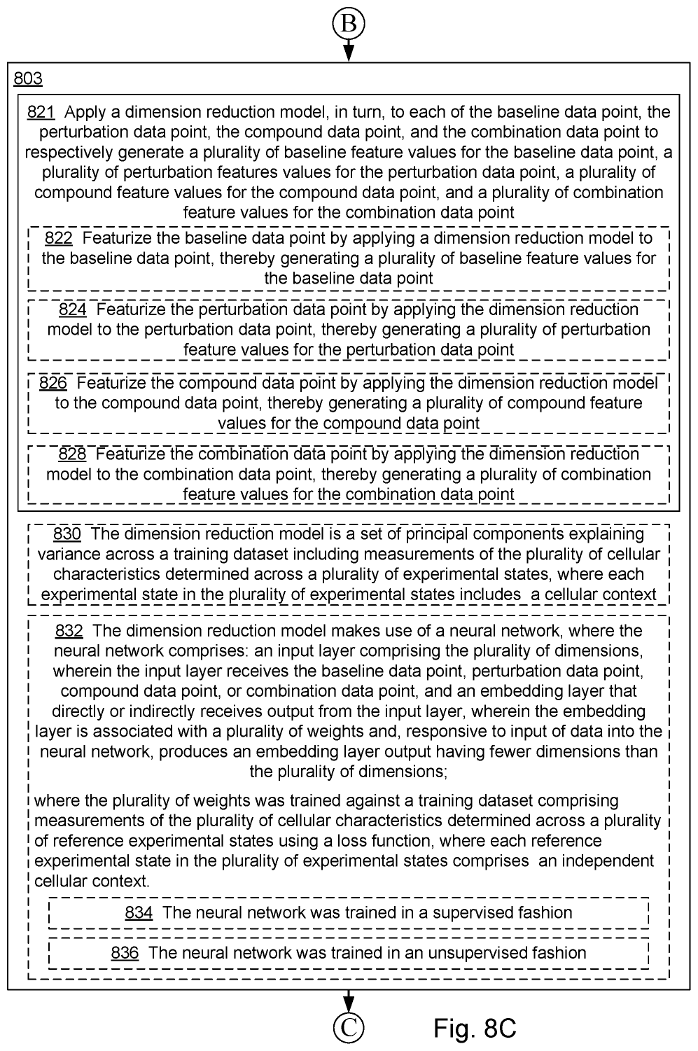

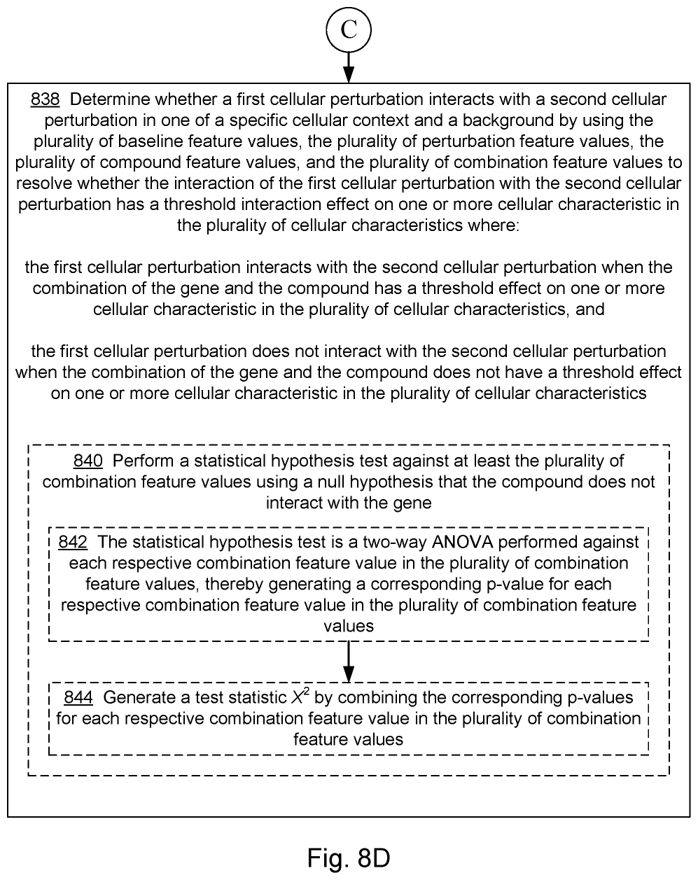

Method 400 begins with a block 801 which is illustrated in FIGS. 8A and 8B. Method 800 includes obtaining (802) a baseline data point for a baseline state (e.g., baseline data point 133, as illustrated in FIGS. 1A, 2C, and 3D, obtained from measurements of cellular characteristics in instances of experimental conditions representative of a baseline state 104). The baseline data point includes a plurality of dimensions, where each respective dimension in the plurality of dimensions of the baseline data point represents a corresponding measure of central tendency of a different cellular characteristic, e.g., in a plurality of cellular characteristics, determined across a plurality of baseline aliquots of cells representing the baseline state, where the baseline state includes a first cellular context.

For instance, each baseline cellular characteristic 113 is measured in each instance of an experimental condition representative of baseline state 104 in the assay (e.g., measurements 113-1-1 through 113-1-16 of the same characteristic are obtained from wells 354-1-1 through 354-1-16, respectively, in FIG. 3B) and then a measure of central tendency is determined across each of the measurements (e.g., the mean of measurements 113-1-1 through 113-1-16 of the first characteristic

$$( {\sum }_{i = 16}^{1} ( \frac{113 - 1 - 1 - 1 - i}{16} ) ) ,$$

as illustrated in FIG. 3D). The measures of central tendency for each of the characteristics representative of the experimental state are then concatenated into a baseline data point (e.g., data point 133) for the respective cellular context. In some embodiments, the measure of central tendency is an arithmetic mean, weighted mean, midrange, midhinge, trimean, geometric mean, geometric median, Winsorized mean, median, or mode of the cellular characteristic.

In some embodiments, each of the cellular characteristics is an optically-measureable characteristic. In other embodiments, at least one cellular characteristic in the plurality of cellular characteristics is measured using a non-optical measurement. Non-limiting examples of optically-measureable and non-optically measureable cellular characteristic are described herein, e.g., in the Cellular Characteristic sections provided below.

In some embodiments, each of the baseline aliquots of cells include a single cell type, e.g., a single type of mammalian cell. In some embodiments, each of the baseline aliquots of cells includes the same mixture of different cell types, e.g., a co-culture of two different mammalian cells. In yet other embodiments, the plurality of baseline aliquots of cells includes different cellular contexts in different instances of the experimental condition that is representative of the baseline state. As described in further detail below, in some embodiments, measurements of the same cellular characteristic are acquired from different cell types in order to account for changes in the characteristic, e.g., upon perturbation of gene expression, exposure to a compound, and/or both, that are specific to one cell type. For instance, referring back to the hypothetical experiment described above with reference to FIGS. 3B-3D, in some embodiments, each baseline experimental condition in wells 354-1-1 to 354-1-16 contains a different cell type, or every two wells across the first row contain a different cell type, or every three wells across the first row contain a different cell type, etc.

In one embodiment, the first cellular context is a mammalian cell line. In one embodiment, the first cellular context is an adherent mammalian cell line (810). In some embodiments, the first cellular context is a human cell. In some embodiments, the first cellular context is a primary human cell. Non-limiting examples of cell types useful for the methods provided herein are described herein, e.g., in the Cell Contexts section provided below.

Method 800 also includes obtaining (804) a perturbation data point for a perturbation state (e.g., perturbation data point 135, as illustrated in FIGS. 1A, 2C, and 3D, obtained from measurements of cellular characteristics in instances of experimental conditions representative of a perturbation state 106). The perturbation data point includes the plurality of dimensions (e.g., the same number of dimensions as the corresponding baseline data point 133), each respective dimension in the plurality of dimensions of the perturbation data point representing the measurement of central tendency of a different cellular characteristic, e.g., in the plurality of cellular characteristics (the same cellular characteristics that were measured for the baseline state), determined across a plurality of perturbation aliquots of cells representing the perturbation state (e.g., referring to the hypothetical example with reference to FIG. 3B, the cellular characteristics are measured for each well 354-2 across the second row of multiwell plate 352, each of which contains an instance of an experimental condition representative of the perturbation state).

The perturbation state includes a first perturbation of the first cellular context in which the expression of a gene is perturbed relative to the expression of the gene in the baseline state. That is, the background for the cellular context(s) used in the perturbation experimental conditions is the same as the cellular context used in the background experimental conditions. However, the expression of a target gene in the background cellular context is perturbed relative to the expression of the target gene in the baseline cellular contexts. As described with reference to the baseline state above, in some embodiments, the same cellular context is used in each of the perturbation experimental conditions (e.g., when the same cellular context is used in each of the baseline experimental conditions). In other embodiments, different cellular contexts are used in different instances of the perturbation experimental conditions (e.g., when different cellular contexts are used in different instances of the baseline experimental conditions). The point is that it is advantageous to use as close to the same cellular background, as possible, in the experimental conditions corresponding to the baseline state and the experimental conditions corresponding to the perturbation state, so that differences in the cellular characteristics of the perturbation state, relative to the baseline state, can be confidently attributable to the perturbation of the target gene.

As outlined above, because the methods provided herein are not tied to any particular cellular dysfunction or disease model, the expression of any gene in the cellular context may be perturbed, to identify interactions with that gene and a second biological agent (e.g., another gene, a candidate drug compound, a soluble factor, or a toxin). Moreover, unlike conventional synthetic lethality assays used to identify interactions between biological agents, interactions between agents that do not cause drastic changes in a cellular feature (e.g., like apoptosis) can still be identified, broadening the utility of the methods described herein beyond conventional methodologies for identifying interactions in a complex biological system.

In some embodiments, the expression of the target gene is perturbed, in the perturbation state, by introduction of an siRNA targeting the gene into the first cellular context of the plurality of perturbation aliquots of cells representing the perturbation state (812). For instance, the cells included in wells 354-2-1 through 354-2-16, representing the perturbation state in the hypothetical example illustrated in FIG. 3B, are the same cells included in wells 354-1-1 through 354-1-16, representing the baseline state, except that one or more siRNA directed to the target gene has been introduced into the cells included in wells 354-2-1 through 354-2-16, but not into the cells included in wells 354-1-1 through 354-1-16.

In some embodiments, a single species of siRNA targeting the gene (e.g., siRNA with a single, defined sequence) is introduced into the first cellular context of each respective perturbation aliquot, in the plurality of perturbation aliquots of cells representing the perturbation state (814). That is, in some embodiments, for every gene that interaction data is being queried, a single siRNA sequence is used in each instance of the perturbation state. In some embodiments, a plurality of siRNA targeting the gene is introduced into the first cellular context of each respective perturbation aliquot, in the plurality of perturbation aliquots of cells representing the perturbation state (816). That is, in some embodiments, multiple siRNA sequences are used to perturb the expression of the target gene.

In yet other embodiments, a first species of siRNA targeting the gene is introduced into the first cell context of a first respective perturbation aliquot, in the plurality of perturbation aliquots of cells representing the perturbation state, and a second species of siRNA targeting the gene is introduced into the first cell context of a second respective perturbation aliquot, in the plurality of perturbation aliquots of cells representing the perturbation state (818). That is, in some embodiments, different siRNA molecules that target a different portion (sequence) of the target gene are used in different instances of the perturbation state. For instance, referring to the hypothetical example illustrated in FIG. 3B, a first siRNA directed to a targeted gene is introduced into cells used in well 354-2-1 of plate 352, and a second siRNA directed to a different sequence in the targeted gene is introduced into cells used in well 354-2-2 (or every other well, every third well, etc,), such that the characteristics represented in the resulting perturbation data point 115 are measures of central tendencies of the characteristic measured across cells in which the targeted gene is perturbed using difference siRNA species. It is known that some siRNA perturb the expression of genes other than the target gene. These off-target affects, depending on the extent to which they affect the expression of the off-target gene, may significantly affect the cellular characteristics measured in the assays described herein. By using a plurality of different siRNA sequences directed to the target gene, changes in the cellular characteristics from any particular off-target effect can be averaged out, since different off-target genes would be expected to be affected by different siRNA sequences.

In some embodiments, the expression of the gene is perturbed, in the perturbation state, by introduction of a CRISPR reagent targeting the gene into the first cellular context of the plurality of perturbation aliquots of cells representing the perturbation state (820). For instance, the cells included in wells 354-2-1 through 354-2-16, representing the perturbation state in the hypothetical example illustrated in FIG. 3B, are the same cells included in wells 354-1-1 through 354-1-16, representing the baseline state, except that the target gene has been altered by one or more CRISPR reagents in the cells included in wells 354-2-1 through 354-2-16, but not into the cells included in wells 354-1-1 through 354-1-16. More details with respect to methods for perturbing gene expression are described herein, e.g., in the Gene Expression Perturbation section provided below.

Method 800 also includes obtaining (806) a compound data point for a compound state (e.g., compound data point 137, as illustrated in FIGS. 1A, 2C, and 3D, obtained from measurements of cellular characteristics in instances of experimental conditions representative of a compound state 108). The compound data point includes the plurality of dimensions (e.g., the same number of dimensions as the corresponding baseline data point 133 and perturbation data point 135), each respective dimension in the plurality of dimensions of the compound data point representing the measurement of central tendency of a different cellular characteristic, in the plurality of cellular characteristics (the same cellular characteristics that were measured for the corresponding baseline state and perturbation state), determined across a plurality of compound aliquots of cells representing the compound state (e.g., referring to the hypothetical example with reference to FIG. 3B, the cellular characteristics are measured for each well 354-3 across the third row of multiwell plate 352, each of which contains an instance of an experimental condition representative of the compound state).

The compound state includes a second perturbation of the first cellular context in which the first cellular context is exposed to a compound. That is, the cellular context(s) used in the compound experimental conditions is the same as the cellular context used in the background experimental conditions, as well as the basis for the cellular context used in the corresponding perturbation experimental conditions. However, the cellular context is exposed to a test compound, e.g., a candidate drug, a soluble factor, or a toxin. In some embodiments, the compound is a candidate drug compound, such that the method is for identifying an interaction between a gene and a candidate drug compound. In some embodiments, the compound is a soluble factor, such that the method is for identifying an interaction between a gene and a soluble factor. In some embodiments, the compound is a toxin, such that the method is for identifying an interaction between a gene and a toxin. More details with respect to compounds useful for method 800 are described herein, e.g., in Compound Perturbation section provided below.

Method 800 also includes obtaining (808) a combination data point for a combination state (e.g., combination data point 139, as illustrated in FIGS. 1A, 2C, and 3D, obtained from measurements of cellular characteristics in instances of experimental conditions representative of a combination state 110). The combination data point includes the plurality of dimensions (e.g., the same number of dimensions as the corresponding baseline data point 133, perturbation data point 135, and compound data point 137), each respective dimension in the plurality of dimensions of the combination data point representing the measurement of central tendency of a different cellular characteristic, in the plurality of cellular characteristics (the same cellular characteristics that were measured for the corresponding baseline state, perturbation state, and compound state), determined across a corresponding plurality of combination aliquots of cells representing the combination state (e.g., referring to the hypothetical example with reference to FIG. 3B, the cellular characteristics are measured for each well 354-4 across the fourth row of multiwell plate 352, each of which contains an instance of an experimental condition representative of the combination state).

The combination state includes a third perturbation of the first cellular context in which (i) the expression of the gene is perturbed relative to the expression of the gene in the baseline state (as in the corresponding perturbation state) and (ii) the first cellular context is exposed to the compound (the same compound as was exposed to the compound state). As described above, with reference to the corresponding perturbation state, expression of the target gene may be perturbed in any number of fashions, e.g., siRNA knock-down with a single siRNA species, a plurality of siRNA species, or different siRNA species in difference instances of the experimental condition. However, it is desirable that the methodology used to perturb the target gene expression be the same as the methodology used in the perturbation state, such that any difference in the measured cellular characteristics, relative to the perturbation state, attributable to the interaction between the targeted gene and the compound, can more easily be identified. Similarly, as described herein with reference to the compound state, the concentration of the test compound may be selected based on various known or expected properties of the compound. However, it is desirable that the concentration of the test compound in the combination state be the same as the concentration of the test compound used in the compound state, such that any difference in the measured cellular characteristics, relative to the compound state, attributable to the interaction between the targeted gene and the compound, can more easily be identified.

Method 800 proceeds to a block 803 illustrated in FIG. 8C. Method 800 includes applying (821) a dimension reduction model, in turn, to each of the baseline data point, the perturbation data point, the compound data point, and the combination data point to respectively generate a plurality of baseline feature values for the baseline data point, a plurality of perturbation features values for the perturbation data point, a plurality of compound feature values for the compound data point, and a plurality of combination feature values for the combination data point. In some embodiments, this may be carried out as described in 822-826 and referred to as “featurizing the data points.”

In method 800 featurizing the data points obtained above (e.g., baseline data point 133, perturbation data point 135, compound data point 137, and combination data point 139), is accomplished to reduce the dimensionality of the data and improve upon any scarcity in the data, e.g., as described above with reference to step 140 in FIG. 1A. As described herein, the lower dimensionality of the featurized data point decreases the processing time required to analyze the data, thereby creating a more efficient processing system 200. Additionally, in some embodiments, featurizing the data reduces or removes multicollinearity between cellular characteristics, which are intercorrelations or inter-associations among independent variables in a data set. Multicollinerarity can cause problems during data analysis that result in models with high errors, e.g., due to poorly estimated partial regression coefficients during regression analysis. Accordingly, featurizing the data can both improve the speed of the process, as well as the results.

Method 800 includes featurizing (822) the baseline data point (e.g., baseline data point 133) by applying a dimension reduction model to the baseline data point, thereby generating a plurality of baseline feature values for the baseline data point. The plurality of baseline feature values define a baseline featurized vector (e.g., baseline feature values F_{B1} through F_{Bn} of baseline featurized data point 143) that has fewer dimensions than the corresponding data point (e.g., baseline data point 133).

Method 800 includes featurizing (824) the perturbation data point (e.g., perturbation data point 135) by applying the dimension reduction model (the same model as used to featurize baseline data point 133) to the perturbation data point, thereby generating a plurality of perturbation feature values for the perturbation data point. The plurality of perturbation feature values define a perturbation featurized vector (e.g., perturbation feature values F_{P1} through F_{Pn} of perturbation featurized data point 145) that has fewer dimensions than the corresponding data point (e.g., perturbation data point 135).

Method 800 includes featurizing (826) the compound data point (e.g., compound data point 137) by applying the dimension reduction model (the same model as used to featurize baseline data point 133 and perturbation data point 135) to the compound data point, thereby generating a plurality of compound feature values for the compound data point. The plurality of compound feature values define a compound featurized vector (e.g., compound feature values F_{D1} through F_{Dn} of compound featurized data point 147) that has fewer dimensions than the corresponding data point (e.g., compound data point 137).

Method 800 includes featurizing (828) the combination data point (e.g., combination data point 139) by applying the dimension reduction model (the same model as used to featurize baseline data point 133, perturbation data point 135, and compound data point 137) to the combination data point, thereby generating a plurality of combination feature values for the combination data point. The plurality of combination feature values define a combination featurized vector (e.g., combination feature values F_{C1} through F_{Cn} of combination featurized data point 149) that has fewer dimensions than the corresponding data point (e.g., combination data point 139).

Featurization of the cellular characteristics is performed by a dimensional reduction technique that uses a statistical feature selection or feature extraction procedure known in the art, for example, principal component analysis, non-negative matrix factorization, kernel PCA, graph-based kernel PCA, linear discriminant analysis, generalized discriminant analysis, and use of an autoencoder. This, in turn, reduces the computational burden of analyzing the data set by compressing the data in order to make the method more computationally efficient, e.g., by allowing the computer to apply an algorithm to the smaller dataset rather than the full dataset. Further information about featurization is described herein, e.g., in the Dimension Reduction section provided below.

In some embodiments, the dimension reduction model is a set of principal components (830) explaining variance across a training dataset including measurements of the plurality of cellular characteristics determined across a plurality of experimental states, where each experimental state in the plurality of experimental states includes a cellular context. For instance, in some embodiments, a set of principal components is learned against a training data set that includes measurements of the same cellular characteristics as used in method 800. In some embodiments, the training data set includes data derived from measurements of cellular characteristics from a plurality of instances of baseline states, e.g., unperturbed cell contexts. In some embodiments, the training data set includes data derived from measurements of cellular characteristics from a plurality of instances of perturbation states, e.g., cell contexts in which the expression of one or more target genes is perturbed, relative to a corresponding unperturbed baseline cellular context. In some embodiments, the training data set includes data derived from measurements of cellular characteristics from a plurality of instances of compound states, e.g., cell contexts which are exposed to one or more test compounds (e.g., candidate drugs, soluble factors, and/or toxins). In some embodiments, the training data set includes data derived from measurements of cellular characteristics from a plurality of instances of combination states, e.g., cell contexts in which the expression of one or more target genes is perturbed, relative to a corresponding unperturbed baseline cellular context, and is exposed to one or more test compounds (e.g., candidate drugs, soluble factors, and/or toxins). In some embodiments, the training data set includes data derived from measurements of cellular characteristics from a plurality of instances of baseline states and a plurality of instances of perturbation states and/or compound states. In some embodiments, the training data set includes data derived from measurements of cellular characteristics from a plurality of instances of baseline states, a plurality of instances of perturbation states, a plurality of instances of compound states, and a plurality of instances of combination states.

In some embodiments, the dimension reduction model makes use of a neural network (832), (e.g., as illustrated in FIG. 9) where the neural network includes: (i) an input layer comprising the plurality of dimensions (e.g., input layer 902), where the input layer receives the baseline data point (e.g., baseline data point 133), perturbation data point (e.g., perturbation data point 135), compound data point (e.g., compound data point 137), or combination data point (e.g., combination data point 139), and (ii) an embedding layer (e.g., embedding layer 910) that directly or indirectly receives output from the input layer. The embedding layer is associated with a plurality of weights (e.g., applied via connections 908) and, responsive to input of data into the neural network, produces an embedding layer (e.g., embedding layer 910) output having fewer dimensions than the plurality of dimensions (e.g., input layer 902, illustrated in FIG. 9, has m-dimensions, while embedding layer 910 has n-dimensions, where m>n). The plurality of weights (e.g., used in neural network 900) was trained against a training dataset including measurements of the plurality of cellular characteristics determined across a plurality of reference experimental states using a loss function, where each reference experimental state in the plurality of experimental states comprises an independent cellular context. For instance, in some embodiments, a neural network (e.g., neural network 900) is trained against a training data set that includes measurements of the same cellular characteristics as used in method 800.

In some embodiments, the training data set includes data derived from measurements of cellular characteristics from a plurality of instances of baseline states, e.g., unperturbed cell contexts. In some embodiments, the training data set includes data derived from measurements of cellular characteristics from a plurality of instances of perturbation states, e.g., cell contexts in which the expression of one or more target genes is perturbed, relative to a corresponding unperturbed baseline cellular context. In some embodiments, the training data set includes data derived from measurements of cellular characteristics from a plurality of instances of compound states, e.g., cell contexts which are exposed to one or more test compounds (e.g., candidate drugs, soluble factors, and/or toxins). In some embodiments, the training data set includes data derived from measurements of cellular characteristics from a plurality of instances of combination states, e.g., cell contexts in which the expression of one or more target genes is perturbed, relative to a corresponding unperturbed baseline cellular context, and is exposed to one or more test compounds (e.g., candidate drugs, soluble factors, and/or toxins). In some embodiments, the training data set includes data derived from measurements of cellular characteristics from a plurality of instances of baseline states and a plurality of instances of perturbation states and/or compound states. In some embodiments, the training data set includes data derived from measurements of cellular characteristics from a plurality of instances of baseline states, a plurality of instances of perturbation states, a plurality of instances of compound states, and a plurality of instances of combination states.

In some embodiments, e.g., referring to FIG. 9, neural network 900 includes an input layer 902 including a plurality of dimensions (e.g., the same number of dimensions as data points 133, 135, 137, and 139, where the input layer receives the baseline data point, perturbation data point, compound data point, or combination data point. For example, as illustrated in FIG. 9, each dimension of input layer 902 receives a term Cᵢ of combination data point 139 (e.g., as illustrated in FIG. 1A). Neural network 900 also includes embedding layer 910 linked to input layer 902 directly or indirectly. For instance, in some embodiments, neural network 900 includes one or more hidden layers 906 positioned between input layer 902 and embedding layer 910 (e.g., coupled via connections 904 and 908). In other embodiments, there are no hidden layers between input layer 902 and embedding layer 910, such that embedding layer 910 receives the output of input layer 902 directly. Embedding layer 910 includes fewer dimensions (e.g., n dimensions) than input layer 902 (e.g., m dimensions, where m>n). Neural network 900 also includes output layer 918 that directly or indirectly receives the output of embedding layer 910. For instance, in some embodiments, neural network 900 includes one or more hidden layers 914 positioned between embedding layer 910 and output layer 918 (e.g., coupled via connections 912 and 916). In other embodiments, there are no hidden layers between embedding layer 910 and output layer 918, such that output layer receives the output of embedding layer 910 directly (e.g., via connections 916). In some embodiments, output layer 918 predicts an attribute of a test experimental state that includes a cellular context, responsive to loading a test data point that includes the plurality of dimensions and is representative of the test state (e.g., a data point of measures of central tendency of cellular characteristics measured across a plurality of instances of the test state). In some embodiments, the neural network is the neural network was trained against a training dataset comprising measurements of the plurality of cellular characteristics determined across a plurality of reference experimental states, wherein each reference experimental state in the plurality of experimental states comprises an independent cellular context, e.g., as described above. The portion of the neural network that serves as the dimension reduction model consists of the input layer (e.g., input layer 902), the embedding layer (e.g., embedding layer 910), and all hidden layers (e.g., optional hidden layer 906) linking the input layer to the embedding layer, such that the output of the embedding layer are the baseline feature values, perturbation feature values, compound feature values, or combination feature values (e.g., as illustrated in FIG. 9, each dimension of input layer 902 receives a term Cᵢ of combination data point 139 and each layer of embedding layer 910 outputs a term F_{ci} of combination state featurized vector 149).

Methods for training neural networks are generally known in the art, e.g., using back propagation techniques, such as stochastic gradient descent. Accordingly, in some embodiments, neural network is trained in a supervised fashion (834). In other embodiments, e.g., where the neural network is an autoencoder, the neural network is trained in an unsupervised fashion (834). For more information regarding artificial neural networks, see, for example, Abiodun O I, et al., Heliyon, 4(11):e00938 (2018), the content of which is incorporated herein by reference.

With reference to FIG. 8D, method 800 then includes determining (838) whether a first cellular perturbation interacts with a second cellular perturbation in one of a specific cellular context and a background by using the plurality of baseline feature values (e.g., baseline featurized data point 143), the plurality of perturbation feature values (e.g., perturbation featurized data point 145), the plurality of compound feature values (e.g., compound featurized data point 147), and the plurality of combination feature values (e.g., combination featurized data point 149) to resolve whether a first cellular perturbation interacts with a second cellular perturbation in one of a specific cellular context and a background. The first cellular perturbation interacts with the second cellular perturbation when the combination of the gene and the compound has a threshold effect on one or more cellular characteristic in the plurality of cellular characteristics. The first cellular perturbation does not interact with the second cellular perturbation when the combination of the gene and the compound does not have a threshold effect on one or more cellular characteristic in the plurality of cellular characteristics.

In some embodiments, a statistical hypothesis test, using the feature values derived from the cell assay data, is performed (840) to determine whether the compound interacts with the gene. In some embodiments, the statistical hypothesis test is performed (840) against at least the plurality of combination feature values using a null hypothesis that the compound does not interact with the gene. In some embodiments, the statistical hypothesis test is a two-way ANOVA performed (842) against each respective combination feature value in the plurality of combination feature values, thereby generating a corresponding p-value for each respective combination feature value in the plurality of combination feature values. For instance, in some embodiments, a two-way ANOVA is performed against each feature F_{ci} of combination featurized data set 149, using corresponding features F_{Bi} of baseline featurized data set 143, F_{Pi} of perturbation featurized data set 145, and F_{Bi} of compound featurized data set 147, thereby generating a corresponding p-value 159 for each feature F_{ci} of combination featurized data set 149.

In some embodiments, determining whether the compound interacts with a gene includes generating (844) a test statistic X² by combining the corresponding p-values (e.g., p-values 159) for each respective combination feature value (e.g., F_{ci}) in the plurality of combination feature values (e.g., featurized data set 149). Methods of meta-analysis combining p-values are known in the art and include, for example, Fischer's method, Pearson's method, George's method, Edgington's method, Stouffer's method, Tippett's method, use of a Beta distribution, use of a truncated gamma distribution, and use of other general distribution functions. For more information regarding methods for combining p-values, and rationales for choosing a particular meta-analysis method, see, for example, Heard N A, “Choosing Between Methods of Combining p-values,” arXiv:1707.06897v4 [stat.ME] 14 Dec. 2017, the content of which is hereby incorporated by reference.

### Cell Contexts

As described above, the baseline states, perturbation states, compound states, and combination states described herein refer to experimental conditions including an aliquot of cells of one or more cellular contexts, which may or may not be perturbed relative to a reference cellular context, and a chemical environment, which may or may not be exposed to one or more test compounds (e.g., candidate drugs, soluble factors, or toxins). In some embodiments, each experimental well receives an aliquot of a single cell type. That is, only one cell type is deposited into a single well, however, different experimental wells may receive different cell types. In some embodiments, one or more experimental wells receives an aliquot of cells containing multiple cell types, e.g., two, three, four, five, six, or more cell types. However, when two experimental conditions are being compared to each other, the cell types (either single cell type or a mixture of cell types) used for each experimental condition are generally the same, such that the only variabilities introduced into the experiment relate to the perturbation of the selected cell type(s).

That is not to say that each well in a particular experimental state necessarily receives the same cell type. In some embodiments, as described above, an experimental state is represented by an average of a plurality of experimental conditions. For instance, in some embodiments, one or more different cell type is used in one or more different wells that correspond to a particular experimental state, and the cellular characteristics of the experimental state are defined by an average of measured characteristics across all wells corresponding to that experimental condition. For instance, referring back to the hypothetical experiment described above with reference to FIGS. 3B-3D, in some embodiments, each baseline experimental condition in wells 354-1-1 to 354-1-16 contains a different cell type, or every two wells across the first row contain a different cell type, or every three wells across the first row contain a different cell type, etc. However, in some embodiments, where different cell types are used across a set of experimental conditions, such as described for wells 354-1-1 to 354-1-16, the same distribution of different cell types is used for a corresponding set of experimental conditions defining an experimental state that will be compared to the previous experimental state. For instance, returning to the hypothetical example described with reference to FIGS. 3B-3D, where every baseline experimental condition in wells 354-1-1 to 354-1-16 includes a different cell type, the set of perturbation experimental condition in wells 354-2-1 to 354-2-16 will also include the same different cell type in each well. Such that, when the characteristics of the baseline state and the characteristics of the perturbation state are averaged over each of the wells representing the respective state, the only variable contributing to differences between the two states is the gene expression perturbation of the different cells types in the perturbation experimental conditions. In this fashion, effects that are specific to one cell type can be averaged out over a plurality of cell types.

In some embodiments, a cell context is one or more cells that have been deposited within a well of a multiwell plate 102, such as a particular cell line, primary cells, or a co-culture system. In some embodiments, as described herein with reference to FIG. 3, in some embodiments, a compound (e.g., a candidate drug, soluble factor, or toxin) is exposed to a plurality of different cell contexts, e.g., at least two, three, four, five, six, seven, eight, nine, ten, or more cell contexts. Likewise, in some embodiments, the expression of a gene is perturbed in a plurality of different cell contexts, e.g., at least two, three, four, five, six, seven, eight, nine, ten, or more cell contexts.

Examples of cell types that are useful for the methods described herein include, but are not limited to, U2OS cells, A549 cells, MCF-7 cells, 3T3 cells, HTB-9 cells, HeLa cells, HepG2 cells, HEKTE cells, SH-SY5Y cells, HUVEC cells, HMVEC cells, primary human fibroblasts, and primary human hepatocyte/3T3-J2 fibroblast co-cultures. In some embodiments a cell line used as a basis for a cell context is a culture of human cells. In some embodiments, a cell line used as a basis for a cell context is any cell line set forth in Table 1 below, or a genetic modification of such a cell line. In some embodiments each cell line used as a different cell context in a particular experimental set-up is from the same species. In some embodiments the cell lines used for a cell context in a particular experimental set-up are from more than one species. For instance, a first cell line used as a first context is from a first species (e.g., human) and a second cell line used as a second context is from a second species (e.g., monkey).

TABLE 1
Example cell types used as a basis for providing
cell context in some embodiments.

| Cell Name | Tissue Type | Tissue | Phenotype | Primary |
|---|---|---|---|---|
| jb6 p+ c141 | Mouse | Skin | Adherent | no |
| jcam1.6 | Human | Lymphocyte | Suspension | no |
| jb6 rt101 | Mouse | Epithelial | Either | yes |
| jy | Human | Lymphocyte | Suspension | no |
| k562 | Human | Bone | Suspension | no |
| j82 | Human | Bladder | Adherent | no |
| ivec cells | Human | Endothelial | Adherent | no |
| jeg-3 | Human | Other | Adherent | no |
| jurkat | Human | Lymphocyte | Suspension | no |
| j558l | Mouse | Blood | Suspension | no |
| k46 | Mouse | Lymphocyte | Suspension | no |
| j774 cells | Mouse | Macrophage | Adherent | no |
| knrk | Rat | Epithelial | Either | no |
| keratinocytes | Mouse | Keratinocyte | Adherent | yes |
| kc1 | Drosophila | Default | Adherent | no |
| | Melanogaster | | | |
| kc18-2-40 cells | Human | Keratinocyte | Adherent | no |
| kt-3 | Human | Lymphocyte | Suspension | no |
| kmst-6 | Human | Skin | Adherent | no |
| l1210-fas | Mouse | Myoblast | Suspension | yes |
| kb | Human | Fibroblast | Adherent | no |
| keratinocytes | Human | Keratinocyte | Adherent | yes |
| kg-1 cells | Human | Bone marrow | Suspension | no |
| ks cells | Human | Skin | Adherent | yes |
| kd83 | Mouse | Blood | Suspension | no |
| l-m(tk-) | Mouse | Connective | Adherent | no |
| l8 cells | Rat | Myoblast | Adherent | yes |
| lk35.2 | Mouse | Lymphocyte | Suspension | no |
| l1210 | Mouse | Monocyte | Suspension | yes |
| lan-5 | Human | Brain | Adherent | no |
| llc-pk1 | Pig | Kidney | Adherent | no |
| lewis lung carcinoma, | Mouse | Lung | Either | no |
| llc | | | | |
| l6e9 | Rat | Muscle | Adherent | no |
| lmh | Chicken | Liver | Adherent | no |
| l6 cells | Rat | Muscle | Adherent | no |
| lisn c4 (nih 3t3 | Mouse | Fibroblast | Adherent | yes |
| derivative | | | | |
| overexpressing egf) | | | | |
| lap1 | Mouse | Lymphocyte | Suspension | yes |
| lap3 | Mouse | Embryo | Adherent | no |
| l929 | Mouse | Fibroblast | Adherent | no |
| mg87 | Mouse | Fibroblast | Adherent | no |
| min6 | Mouse | Default | Either | no |
| mel | Mouse | Other | Adherent | no |
| melenoma cells | Human | Melanoma | Adherent | yes |
| mdbk | Cow | Kidney | Adherent | no |
| mkn45 gastric cancer | Human | Stomach | Adherent | yes |
| mewo | Human | Melanoma | Adherent | no |
| mda-mb-468 | Human | Breast/Mammary | Adherent | no |
| mdck | Dog | Kidney | Adherent | no |
| mf4/4 | Mouse | Macrophage | Adherent | no |
| me-180 | Human | Cervix | Adherent | yes |
| mes-sa | Human | Uterus | Adherent | no |
| mg-63 cells | Human | Bone | Adherent | no |
| mono-mac-6 cells | Human | Blood | Suspension | no |
| monocytes | Human | Blood | Suspension | yes |
| mrc-5 | Human | Lung | Adherent | yes |
| mob cells | Mouse | Osteoblast | Adherent | yes |
| msc human | Human | Bone marrow | Adherent | yes |
| mesenchymal stem cell | | | | |
| mt-2 | Human | Lymphocyte | Adherent | yes |
| mouse embryonic | Mouse | Fibroblast | Adherent | yes |
| fibroblasts | | | | |
| mnt1 | Human | Skin | Adherent | yes |
| ms1 | Mouse | Pancreas | Adherent | no |
| mr1 | Rat | Embryo | Adherent | no |
| mt4 | Human | Lymphocyte | Suspension | yes |
| molt4 (human acute t | Human | Blood | Suspension | no |
| lymphoblastic | | | | |
| leukaemia) | | | | |
| hep3b | Human | Liver | Adherent | no |
| hepatic stellate cells | Rat | Liver | Adherent | yes |
| hela 229 cells | Human | Cervix | Either | yes |
| hep2 | Human | Epithelial | Adherent | no |
| hela-cd4 | Human | Epithelial | Adherent | no |
| hct116 | Human | Colon | Adherent | no |
| hepatocytes | Mouse | Liver | Adherent | yes |
| hela s3 | Human | Cervix | Adherent | no |
| hel | Human | Lymphocyte | Suspension | yes |
| hela cells | Human | Cervix | Adherent | no |
| hela t4 | Human | Blood | Suspension | no |
| hepg2 | Human | Liver | Adherent | no |
| high 5 (bti-tn-5b1-4) | Insect | Embryo | Adherent | no |
| hit-t15 cells | Hamster | Epithelial | Adherent | no |
| hepatocytes | Rat | Liver | Adherent | yes |
| hitb5 | Human | Muscle | Adherent | yes |
| hi299 | Human | Lung | Adherent | no |
| hfff2 | Human | Foreskin | Adherent | yes |
| hib5 | Rat | Brain | Adherent | yes |
| hm-1 embryonic stem | Mouse | Other | Adherent | yes |
| cells | | | | |
| hitb5 | Human | Muscle | Adherent | yes |
| hl-60 | Human | Lymphocyte | Suspension | no |
| hl-5 | Mouse | Heart | Adherent | no |
| hl-1 | Mouse | Heart | Adherent | no |
| glya | Hamster | Ovary | Adherent | no |
| gamma 3t3 | Mouse | Fibroblast | Adherent | no |
| gh3 | Rat | Pituitary | Adherent | no |
| granta-519 | Human | Blood | Suspension | no |
| freestyle 293 | Human | Kidney | Suspension | no |
| g401 | Human | Connective | Adherent | no |
| fto-2b (rat hepatoma) | Rat | Liver | Suspension | yes |
| cells | | | | |
| gh4c1 | Rat | Pituitary | Adherent | yes |
| fsdc, murine dendritic | Mouse | Blood | Either | no |
| cell | | | | |
| goto | Human | Neuroblastoma | Adherent | yes |
| gc-2spd (ts) | Mouse | Epithelial | Adherent | no |
| glomeruli | Rat | Lung | Adherent | yes |
| frt | Rat | Thyroid | Suspension | no |
| h19-7/igf-ir | Rat | Brain | Suspension | no |
| gt1 | Mouse | Brain | Adherent | no |
| griptite? 293 msr | Human | Kidney | Adherent | no |
| h441 | Human | Lung | Adherent | yes |
| h-500, leydig tumor cell | Rat | Testes | Adherent | yes |
| h4 | Human | Glial | Adherent | no |
| guinea pig endometrial | Guinea Pig | Ovary | Adherent | yes |
| stromal cells | | | | |
| h187 | Human | Lung | Adherent | yes |
| h35 | Rat | Liver | Adherent | no |
| h-7 | Mouse | Bone marrow | Suspension | no |
| h1299 | Human | Lung | Adherent | no |
| granulosa cells | Mouse | Ovary | Either | yes |
| hbl100 cells | Human | Breast/Mammary | Adherent | no |
| h9c2 | Rat | Myoblast | Adherent | no |
| hbec-90 | Human | Brain | Adherent | no |
| has-p | Mouse | Breast/Mammary | Adherent | yes |
| hasmcs | Human | Muscle | Adherent | no |
| hc11 | Mouse | Breast/Mammary | Adherent | no |
| hacat | Human | Keratinocyte | Adherent | yes |
| hb60-5 cells | Mouse | Spleen | Adherent | no |
| h4iie | Rat | Liver | Adherent | yes |
| hca-7 | Human | Colon | Adherent | yes |
| hcd57 | Mouse | Blood | Suspension | no |
| haecs | Human | Aorta | Adherent | yes |
| rpe.40 | Hamster | Kidney | Adherent | yes |
| rcme, rabbit coronary | Rabbit | Endothelial | Adherent | yes |
| microvessel endothelial | | | | |
| rko, rectal carcinoma | Human | Colon | Adherent | no |
| cell line | | | | |
| ros, rat osteoblastic cell | Rat | Osteoblast | Adherent | yes |
| line | | | | |
| rh18 | Human | Muscle | Adherent | no |
| rcho | Rat | Default | Adherent | no |
| rccd1 | Rat | Kidney | Adherent | no |
| s194 cells | Mouse | Lymphocyte | Adherent | yes |
| rin 1046-38 | Rat | Pancreas | Suspension | no |
| rw-4 | Mouse | Embryo | Adherent | yes |
| rj2.2.5 | Human | Lymphocyte | Suspension | no |
| rk13 | Rabbit | Kidney | Adherent | no |
| remc | Rat | Breast/Mammary | Adherent | no |
| sk-br-3 | Human | Breast/Mammary | Adherent | no |
| s49.1 | Mouse | Thymus | Suspension | no |
| schizosaccharomyces | Yeast | Other | Either | yes |
| pombe | | | | |
| sf9 | Insect | Ovary | Suspension | no |
| sf21 | Insect | Other | Either | yes |
| sf21ae | Insect | Other | Either | yes |
| sh-sy5y | Human | Brain | Either | no |
| s2-013 | Human | Pancreas | Either | yes |
| saos-2 | Human | Bone | Adherent | no |
| siha | Human | Cervix | Adherent | no |
| scc12, human squamous | Human | Skin | Adherent | yes |
| cell carcinoma line | | | | |
| (c12c20) | | | | |
| shep | Human | Brain | Adherent | no |
| sk-lms-1 | Human | Other | Adherent | no |
| sk-n-sh, neuronal cells | Human | Brain | Adherent | yes |
| sk-n-as | Human | Neuroblastoma | Adherent | no |
| sknmc | Human | Brain | Adherent | no |
| sk-hep-1 cells | Human | Skin | Either | yes |
| skov3 | Human | Ovary | Adherent | no |
| sk-n-be(2) | Human | Neuroblastoma | Adherent | yes |
| smmc7721 | Human | Liver | Adherent | no |
| smooth muscle cells | Rat | Aorta | Adherent | yes |
| (aortic) rasmc (a7-r5) | | | | |
| sl2 | Drosophila | Default | Either | no |
| | melanogaster | | | |
| sk-ut-1 | Human | Muscle | Adherent | no |
| n2a | Mouse | Neuroblastoma | Adherent | no |
| myocytes (ventricular) | Rat | Heart | Adherent | yes |
| mtln3 | Rat | Breast/Mammary | Adherent | no |
| n1e-115 | Mouse | Brain | Adherent | no |
| mtsv1-7 | Human | Epithelial | Adherent | no |
| murine alveolar | Rat | Lung | Adherent | no |
| macrophages cell line | | | | |
| mhs | | | | |
| n18tg cells | Mouse | Neuroblastoma | Adherent | no |
| n13 | Mouse | Brain | Adherent | no |
| mutu group3, b-cell line | Human | Lymphocyte | Suspension | no |
| mtd-1a | Mouse | Epithelial | Adherent | yes |
| mutu i | Human | Lymphocyte | Suspension | no |
| mv1lu | Mink | Lung | Adherent | no |
| ncb20 | Mouse | Neuroblastoma | Adherent | yes |
| nb324k | Human | Kidney | Adherent | no |
| neural stem cells | Rat | Brain | Either | yes |
| neuroblastoma | Human | Brain | Adherent | yes |
| nci-h23 | Human | Lung | Adherent | no |
| nci-h460 | Human | Lung | Adherent | no |
| neurons (astrocytes) | Rat | Brain | Adherent | yes |
| neuro 2a, a murine | Mouse | Neuroblastoma | Adherent | no |
| neuroblastoma cell line | | | | |
| nbt-ii | Rat | Bladder | Adherent | no |
| neuons (astrocytes) | Rat | Astrocyte | Adherent | yes |
| nci-h295 | Human | Kidney | Adherent | no |
| nci-h358 | Human | Lung | Adherent | no |
| neuons (hippocampal & | Rat | Brain | Adherent | yes |
| septal) | | | | |
| neurons | Mouse | Brain | Adherent | yes |
| nhdf | Human | Fibroblast | Adherent | no |
| neurons (post- | Rat | Brain | Adherent | yes |
| natal/adult) | | | | |
| nhbe | Human | Lung | Adherent | yes |
| ng108-15 | Mouse | Neuroblastoma | Adherent | no |
| neurons (embryonic | Rat | Brain | Adherent | yes |
| cortical) | | | | |
| neurons (cortical) | Mouse | Other | Adherent | yes |
| ng 125 | Human | Neuroblastoma | Adherent | no |
| nhf3 | Human | Fibroblast | Adherent | no |
| neurospora crassa | Fungi | Embryo | Adherent | yes |
| neurons (superior | Rat | Brain | Adherent | yes |
| cervical ganglia - scg) | | | | |
| neurons (ganglia) | Frog | Brain | Either | yes |
| ns20y | Mouse | Neuroblastoma | Adherent | no |
| nrk | Rat | Fibroblast | Adherent | yes |
| nmumg | Mouse | Breast/Mammary | Adherent | no |
| o23 | Hamster | Fibroblast | Adherent | no |
| nt2 | Human | Fibroblast | Adherent | no |
| nhff | Human | Foreskin | Adherent | yes |
| nih 3t3, 3t3-l1 | Mouse | Fibroblast | Adherent | no |
| ohio helas | Human | Cervix | Suspension | no |
| nih 3t6 | Mouse | Fibroblast | Adherent | no |
| nih 3t3-l1, nih 3t3 | Mouse | Embryo | Adherent | no |
| nt2-d1 | Human | Testes | Adherent | no |
| nih 3t3-l1, nih 3t3 ( ) | Mouse | Embryo | Adherent | no |
| orbital fibroblast | Human | Fibroblast | Adherent | yes |
| osteoblasts | Rat | Bone | Adherent | yes |
| p19 cells | Mouse | Embryo | Adherent | yes |
| ovcar-3 | Human | Ovary | Adherent | no |
| opaec cells | Sheep | Endothelial | Adherent | no |
| ovarian surface | Human | Ovary | Adherent | yes |
| epithelial (ose) | | | | |
| p388d1 | Mouse | Macrophage | Adherent | yes |
| p825, mastocytoma cells | Mouse | Macrophage | Adherent | yes |
| p19cl6 | Mouse | Heart | Adherent | no |
| omega e | Mouse | Embryo | Adherent | no |
| ok, derived from renal | Opossum | Kidney | Adherent | yes |
| proximal tubules | | | | |
| p815, mastocytoma cells | Mouse | Macrophage | Adherent | yes |
| p3.653 × ag8 murine | Mouse | Bone marrow | Adherent | yes |
| myeloma cells | | | | |
| paju, human neural | Human | Brain | Adherent | yes |
| crest-derived cell line | | | | |
| pac-1 | Rat | Aorta | Adherent | no |
| parp−/− mouse | Mouse | Fibroblast | Suspension | no |
| embryonic fibroblasts | | | | |
| pci-13 | Human | Skin | Adherent | no |
| pc 6 | Rat | Glial | Adherent | no |
| (pheochromocytoma-6) | | | | |
| pancreatic islets | Rat | Pancreas | Adherent | yes |
| peripheral blood | Human | Blood | Either | yes |
| lymphocytes | | | | |
| pc-3 | Human | Prostate | Either | no |
| pc-12 | Rat | Brain | Adherent | no |
| panc1 | Human | Pancreas | Adherent | no |
| per.c6 ® | Human | Retina | Either | no |
| pa 317 or pt67 mouse | Mouse | Fibroblast | Adherent | yes |
| fibroblast with herpes | | | | |
| thymidine kinase (tk) | | | | |
| gene | | | | |
| pam212, mouse | Mouse | Keratinocyte | Adherent | yes |
| keratinocytes | | | | |
| peripheral blood | Human | Blood | Suspension | yes |
| mononuclear cells | | | | |
| (pbmc) | | | | |
| qt6 | Quail | Fibroblast | Adherent | no |
| pu5-1.8 cells | Mouse | Macrophage | Suspension | no |
| primary lymphoid (oka) | Shrimp | Lymphocyte | Adherent | yes |
| organ from penaeus | | | | |
| shrimp | | | | |
| ps120, an nhe-deficient | Hamster | Lung | Adherent | yes |
| clone derived from | | | | |
| ccl39 cells | | | | |
| phoenix-eco cells | Human | Embryo | Adherent | no |
| quail embryos | Quail | Embryo | Either | yes |
| plb985 | Human | Blood | Suspension | no |
| rabbit pleural | Rabbit | Lung | Adherent | no |
| mesothelial | | | | |
| r1 embryonic stem cell, | Mouse | Embryo | Either | no |
| es | | | | |
| rabbit vsmc, vascular | Rabbit | Muscle | Adherent | yes |
| smooth muscle cells | | | | |
| raec, rat aortic | Rat | Aorta | Adherent | yes |
| endothelial cells | | | | |
| raji | Human | Lymphocyte | Suspension | no |
| rat epithelial cells | Rat | Epithelial | Adherent | yes |
| raw 264.7 cells, murine | Mouse | Macrophage | Adherent | yes |
| macrophage cells | | | | |
| ramos | Human | Lymphocyte | Suspension | no |
| rat hepatic ito cells | Rat | Liver | Adherent | yes |
| rat adipocyte | Rat | Adipose | Adherent | yes |
| rat c5, glioma cells | Rat | Glial | Adherent | yes |
| rat-1, rat fibroblasts | Rat | Fibroblast | Adherent | yes |
| rat 2, rat fibroblasts | Rat | Fibroblast | Adherent | yes |
| rat glomerular mesangial | Rat | Kidney | Adherent | yes |
| mc cells | | | | |
| raw cells | Rat | Peritoneum | Suspension | no |
| rat-6 (r6), rat embryo | Rat | Fibroblast | Adherent | yes |
| fibroblast | | | | |
| hmec-1 | Human | Endothelial | Adherent | yes |
| hre h9 | Rabbit | Uterus | Adherent | no |
| hmn 1 | Mouse | Neuroblastoma | Adherent | yes |
| ht-29 | Human | Colon | Adherent | no |
| hos | Human | Osteoblast | Adherent | no |
| hs68 | Human | Foreskin | Adherent | yes |
| hmcb | Human | Skin | Adherent | no |
| hs-578t | Human | Breast/Mammary | Adherent | no |
| hnscc | Human | Skin | Adherent | no |
| hpb-all | Human | Lymphocyte | Suspension | no |
| hmvec-l | Human | Lung | Adherent | no |
| hsy-eb | Human | Other | Adherent | no |
| huh 7 | Human | Liver | Adherent | no |
| htlm2 | Mouse | Breast/Mammary | Adherent | yes |
| hut 78 | Human | Skin | Suspension | no |
| ht1080 | Human | Fibroblast | Adherent | no |
| huvec, huaec | Human | Umbilicus | Adherent | yes |
| htla230 | Human | Neuroblastoma | Adherent | yes |
| hybridoma | Mouse | Spleen | Suspension | no |
| ib3-1 | Human | Lung | Adherent | no |
| ht22 | Mouse | Brain | Adherent | yes |
| human skeletal muscle | Human | Muscle | Adherent | yes |
| ht4 | Human | Testes | Adherent | yes |
| hutu 80 | Human | Colon | Adherent | yes |
| in vivo mouse brain | Mouse | Bone | Either | yes |
| in vivo rat brain | Rat | Brain | Either | yes |
| iec-6 rie | Rat | Epithelial | Adherent | no |
| imr-32 | Human | Neuroblastoma | Adherent | no |
| ic11 | Mouse | Testes | Adherent | no |
| imr-90 | Human | Lung | Adherent | no |
| in vivo rat lung | Rat | Lung | Either | yes |
| in vivo rat liver | Rat | Liver | Either | yes |
| ins-1 | Rat | Pancreas | Adherent | no |
| in vivo rabbit eye | Rabbit | Other | Either | yes |
| in vivo mouse | Mouse | Other | Either | yes |
| imdf | Mouse | Skin | Adherent | no |
| in vivo pig | Pig | Other | Either | yes |
| caski | Human | Cervix | Adherent | no |
| cerebellar | Mouse | Brain | Adherent | yes |
| cd34+ monocytes | Human | Monocyte | Suspension | yes |
| cfk2 | Rat | Bone | Adherent | no |
| cem | Human | Blood | Suspension | no |
| catha, cath.a | Mouse | Brain | Either | no |
| ccl-16-b9 | Hamster | Lung | Adherent | no |
| ch12f3-2a | Mouse | Lymphocyte | Suspension | no |
| cf2th | Dog | Thymus | Adherent | no |
| cardiomyocytes | Human | Heart | Adherent | yes |
| cg-4 | Rat | Glial | Adherent | no |
| cell.220(b8) | Human | Default | Suspension | no |
| cardiomyocytes | Rat | Heart | Adherent | yes |
| chick embryo fibroblasts | Chicken | Embryo | Adherent | yes |
| chicken sperm | Chicken | Sperm | Adherent | yes |
| cho k1 | Hamster | Ovary | Adherent | no |
| cho 58 | Hamster | Ovary | Adherent | no |
| cho-b7 | Hamster | Ovary | Adherent | no |
| chick embryo | Chicken | Embryo | Adherent | yes |
| blastodermal cells | | | | |
| cho -b53 | Hamster | Ovary | Adherent | yes |
| chick embryo | Chicken | Embryo | Adherent | yes |
| chondrocytes | | | | |
| chinese hamster lung | Hamster | Lung | Adherent | no |
| cho dg44 | Hamster | Ovary | Either | no |
| cho - b53 jf7 | Hamster | Ovary | Adherent | yes |
| chicken hepatocytes | Chicken | Liver | Adherent | yes |
| cos-1 | Primate - Non | Kidney | Adherent | no |
| | Human | | | |
| cho-lec1 | Hamster | Ovary | Adherent | yes |
| clone a | Human | Colon | Adherent | no |
| cho-lec2 | Hamster | Ovary | Adherent | no |
| colo205 | Human | Colon | Adherent | no |
| chu-2 | Human | Epithelial | Adherent | no |
| cmt-93 | Mouse | Rectum | Adherent | no |
| cho-s | Hamster | Ovary | Suspension | no |
| cho-leu c2gnt | Hamster | Ovary | Adherent | no |
| cho-trvb | Hamster | Ovary | Adherent | no |
| clone-13, mutant b | Human | Lymphocyte | Suspension | no |
| lymphoblastoid | | | | |
| cj7 | Mouse | Embryo | Adherent | no |
| smooth muscle cells | Rat | Muscle | Adherent | yes |
| (aortic) | | | | |
| splenocytes | Mouse | Spleen | Suspension | yes |
| smooth muscle cells | Rat | Muscle | Adherent | yes |
| (vascular) | | | | |
| sp1 | Mouse | Breast/Mammary | Adherent | no |
| stem | Rat | Bone | Suspension | yes |
| spoc-1 | Rat | Trachael | Adherent | no |
| snb19 | Human | Brain | Adherent | no |
| splenocytes (resting b | Mouse | Spleen | Suspension | yes |
| cells) | | | | |
| splenocytes (b cells t2) | Mouse | Spleen | Suspension | yes |
| svr | Mouse | Pancreas | Adherent | no |
| stem cells | Human | Bone marrow | Suspension | yes |
| smooth muscle cells | Human | Muscle | Adherent | yes |
| (vascular) | | | | |
| smooth muscle cells | Rabbit | Aorta | Adherent | yes |
| (vascular) | | | | |
| t3cho/at1a | Hamster | Ovary | Either | no |
| t-rex-cho | Hamster | Ovary | Adherent | no |
| t-rex-293 | Human | Kidney | Adherent | no |
| sw620 | Human | Colon | Adherent | no |
| t lymphocytes (t cells) | Mouse | Lymphocyte | Adherent | yes |
| t lymphocytes cytotoxic | Mouse | Lymphocyte | Either | yes |
| (ctl) cells | | | | |
| sw480 | Human | Colon | Adherent | no |
| t lymphocytes (t cells) | Human | Lymphocyte | Adherent | yes |
| sw13 | Human | Adrenal | Adherent | no |
| | | gland/cortex | | |
| t47d, t-47d | Human | Breast/Mammary | Adherent | no |
| t24 | Human | Bladder | Adherent | no |
| t-rex hela | Human | Cervix | Adherent | no |
| tr2 | Mouse | Brain | Adherent | no |
| tig | Human | Fibroblast | Adherent | yes |
| t98g | Human | Brain | Adherent | no |
| tsa201 | Human | Embryo | Adherent | no |
| tobacco protoplasts | Plant | Other | Suspension | yes |
| thp-1 | Human | Blood | Suspension | yes |
| tk.1 | Mouse | Lymphocyte | Suspension | no |
| tib-90 | Mouse | Fibroblast | Adherent | no |
| ta3 | Mouse | Breast/Mammary | Adherent | no |
| tyknu cells | Human | Ovary | Adherent | no |
| u-937 | Human | Macrophage | Suspension | no |
| tgw-nu-1 | Human | Bladder | Adherent | no |
| b-lcl | Human | Blood | Suspension | no |
| b4.14 | Primate - Non | Kidney | Adherent | yes |
| | Human | | | |
| b82 m721 | Mouse | Fibroblast | Adherent | no |
| b-tc3 | Mouse | Pancreas | Adherent | no |
| b16-f10 | Mouse | Melanoma | Adherent | no |
| b82 | Mouse | Fibroblast | Adherent | no |
| as52 | Hamster | Ovary | Adherent | no |
| b lymphocytes | Human | Blood | Suspension | yes |
| b35 | Rat | Neuroblastoma | Adherent | yes |
| b65 | Rat | Neuroblastoma | Adherent | no |
| b11 | Mouse | Spleen | Suspension | no |
| att-20 | Mouse | Pituitary | Adherent | no |
| bcl-1 | Mouse | Lymphocyte | Adherent | no |
| bac | Cow | Adrenal Gland | Adherent | yes |
| balb/c 3t3, 3t3-a31 | Mouse | Fibroblast | Adherent | no |
| be(2)-c | Human | Neuroblastoma | Adherent | no |
| bewo | Human | Other | Adherent | no |
| balb/mk | Mouse | Epithelial | Adherent | no |
| beas-2b | Human | Lung | Adherent | no |
| bewo | Human | Uterus | Adherent | yes |
| baf3, ba/fi | Mouse | Lymphocyte | Suspension | no |
| bcec | Human | Brain | Adherent | yes |
| bc3h1 | Mouse | Brain | Adherent | yes |
| baec | Cow | Aorta | Adherent | no |
| a10 | Rat | Muscle | Adherent | no |
| a1.1 | Mouse | Lymphocyte | Adherent | yes |
| a72 | Dog | Connective | Adherent | no |
| a549 | Human | Lung | Adherent | no |
| a204 | Human | Muscle | Adherent | yes |
| a6 | Frog | Kidney | Adherent | no |
| a875 | Human | Melanoma | Adherent | yes |
| a498 | Human | Kidney | Adherent | no |
| a172 | Human | Brain | Adherent | yes |
| a-431 | Human | Skin | Adherent | no |
| a20 | Mouse | Lymphocyte | Suspension | yes |
| arpe-19 | Human | Retina | Adherent | no |
| alpha t3 | Human | Pituitary | Adherent | no |
| akr | Mouse | Spleen | Adherent | no |
| ar4-2j | Rat | Pancreas | Adherent | no |
| aortic endothelial cells | Human | Aorta | Adherent | yes |
| achn | Human | Kidney | Adherent | yes |
| adventitial fibroblasts | Human | Aorta | Adherent | yes |
| am12 | Mouse | Blood | Suspension | no |
| anterior pituitary | Human | Pituitary | Adherent | yes |
| gonadotropes | | | | |
| ae-1 | Mouse | Spleen | Suspension | no |
| ab1 | Mouse | Embryo | Adherent | no |
| anjou 65 | Human | Default | Either | no |
| crfk | Cat | Kidney | Adherent | no |
| d.mel-2 | Insect | Embryo | Either | no |
| ct26 | Mouse | Colon | Either | yes |
| cowpea plant embryos | Fungi | Embryo | Adherent | yes |
| cos-7 | Primate - Non | Kidney | Adherent | no |
| | Human | | | |
| crl6467 | Mouse | Liver | Adherent | no |
| cwr22rv1 | Human | Prostate | Adherent | no |
| ct60 | Hamster | Ovary | Adherent | no |
| cos-gs1 | Primate - Non | Kidney | Adherent | no |
| | Human | | | |
| cos-m6 | Primate - Non | Kidney | Adherent | yes |
| | Human | | | |
| cv-1 | Primate - Non | Kidney | Adherent | no |
| | Human | | | |
| ctll-2 | Mouse | Lymphocyte | Suspension | no |
| d3 embryonic stem cells | Mouse | Embryo | Adherent | no |
| du145 | Human | Prostate | Adherent | no |
| do-11.10 | Mouse | Lymphocyte | Suspension | no |
| daudi | Human | Lymphocyte | Suspension | no |
| d10 | Mouse | Lymphocyte | Suspension | no |
| dgz | Plant | Other | Adherent | yes |
| dictyostelium | Amoeba | Other | Suspension | yes |
| dt40 | Chicken | Bursa | Suspension | no |
| drosophila kc | Insect | Embryo | Adherent | yes |
| df1 | Chicken | Fibroblast | Adherent | no |
| dc 2.4 cells | Mouse | Blood | Either | no |
| daoy | Human | Other | Adherent | no |
| lovo | Human | Colon | Adherent | no |
| lncap | Human | Prostate | Adherent | no |
| m21 | Human | Melanoma | Adherent | no |
| lsv5 | Human | Keratinocyte | Adherent | no |
| ltk | Mouse | Connective | Adherent | no |
| m1 | Rat | Embryo | Adherent | no |
| m3z | Human | Breast/Mammary | Adherent | no |
| m21-l | Human | Melanoma | Adherent | no |
| lymphoid cell line | Rat | Lymphocyte | Suspension | no |
| m-imcd | Mouse | Kidney | Adherent | yes |
| m12.4 | Mouse | Lymphocyte | Adherent | no |
| m21-14 | Human | Melanoma | Adherent | no |
| mat b iii | Rat | Breast/Mammary | Adherent | no |
| mda-mb-453 | Human | Breast/Mammary | Adherent | no |
| mca-rh7777 | Rat | Liver | Adherent | no |
| ma104 | Primate - Non | Kidney | Adherent | no |
| | Human | | | |
| magi-ccr5 | Human | Epithelial | Adherent | no |
| mda-mb-231 | Human | Breast/Mammary | Adherent | no |
| mcf-10 | Human | Breast/Mammary | Adherent | no |
| mc3t3-e1 | Mouse | Osteoblast | Adherent | no |
| mc ardle 7777 | Rat | Liver | Either | yes |
| macrophages | Mouse | Peritoneum | Adherent | yes |
| mcf-7 | Human | Breast/Mammary | Adherent | no |
| macrophages | Human | Blood | Either | yes |
| maize protoplasts | Plant | Other | Adherent | no |
| umr 106-01 | Rat | Bone | Adherent | no |
| uc729-6 | Human | Lymphocyte | Either | no |
| u9737 | Human | Lymphocyte | Suspension | no |
| uok257 | Human | Kidney | Adherent | no |
| u373mg | Human | Astrocyte | Adherent | no |
| wit49 wilms tumor | Human | Lung | Either | yes |
| vero | Primate - Non | Kidney | Adherent | no |
| | Human | | | |
| u87, u87mg | Human | Astrocyte | Adherent | no |
| umrc6 | Human | Kidney | Adherent | no |
| u251 cells | Human | Glial | Adherent | no |
| u2os | Human | Bone | Adherent | no |
| bovine chromaffin cells | Cow | Adrenal Gland | Adherent | yes |
| bowes melanoma cells | Human | Skin | Adherent | no |
| boll weevil brl-ag-3c | Insect | Other | Adherent | no |
| bm5 | Insect | Ovary | Suspension | no |
| bhk-21 | Hamster | Kidney | Either | no |
| bosc 23 | Human | Kidney | Adherent | yes |
| bms-black mexican | Default | Default | Suspension | yes |
| sweet protoplasts | | | | |
| bfc012 | Mouse | Embryo | Adherent | no |
| bone marrow cells | Mouse | Bone marrow | Suspension | yes |
| bone marrow derived | Human | Bone marrow | Adherent | yes |
| stromal cells | | | | |
| bs-c-1, bsc-1 | Primate - Non | Kidney | Adherent | no |
| | Human | | | |
| bjab | Human | Lymphocyte | Suspension | no |
| bnl cl.2 (cl2) | Mouse | Liver | Adherent | no |
| btm (bovine trachael | Cow | Muscle | Adherent | no |
| myocytes) | | | | |
| c2c12 | Mouse | Muscle | Adherent | no |
| c3a | Human | Liver | Adherent | no |
| c1.39t | Human | Fibroblast | Adherent | no |
| bt cells | Cow | Fibroblast | Adherent | no |
| bsc-40 | Primate - Non | Kidney | Adherent | no |
| | Human | | | |
| c33 | Human | Cervix | Adherent | no |
| c1c12 | Mouse | Muscle | Adherent | no |
| c127 | Mouse | Epithelial | Adherent | no |
| bt549 | Human | Breast/Mammary | Adherent | no |
| c1r, hmy2.c1r | Human | Lymphocyte | Adherent | yes |
| c13-nj | Human | Glial | Adherent | no |
| canine gastric parietal | Dog | Stomach | Adherent | yes |
| cells | | | | |
| calu-3 | Human | Lung | Adherent | yes |
| cak | Mouse | Fibroblast | Adherent | no |
| c57bl/6 cells | Mouse | Heart | Adherent | no |
| caco-2 cells | Human | Colon | Adherent | no |
| c3h 10t1/2 | Mouse | Fibroblast | Adherent | no |
| ca77 | Rat | Thyroid | Adherent | no |
| c6 cells | Rat | Brain | Adherent | no |
| calu-6 | Human | Lung | Adherent | no |
| capan-2 | Human | Pancreas | Adherent | no |
| c4-2 | Human | Prostate | Adherent | no |
| 143b | Human | Bone marrow | Either | no |
| 1064sk | Human | Foreskin | Adherent | yes |
| 16-9 | Human hamster | Other | Adherent | no |
| | hybrid cell line - | | | |
| | transfected with | | | |
| | two human genes | | | |
| 2008 | Human | Ovary | Adherent | no |
| 208f | Rat | Fibroblast | Adherent | no |
| 293-h | Human | Kidney | Either | no |
| 293 | Human | Kidney | Either | no |
| 293 ebna | Human | Kidney | Adherent | no |
| 293t | Human | Kidney | Either | no |
| 2pk3 | Mouse | Lymphocyte | Suspension | no |
| 293-f | Human | Kidney | Either | no |
| 2780 | Human | Ovary | Adherent | no |
| 293s | Human | Kidney | Either | no |
| 2774 | Human | Ovary | Adherent | no |
| 3y1 | Rat | Fibroblast | Adherent | yes |
| 82-6 | Human | Fibroblast | Adherent | no |
| 9hte | Human | Trachael | Adherent | yes |
| 3.12 | Mouse | Lymphocyte | Either | yes |
| 5637 | Human | Bladder | Adherent | no |
| 4t1 | Mouse | Breast/Mammary | Adherent | no |
| 3t3-f442a | Mouse | Other | Adherent | yes |
| 33.1.1 | Mouse | Lymphocyte | Suspension | no |
| 32d | Mouse | Bone marrow | Either | no |
| 4de4 | Mouse | Bone marrow | Either | yes |
| e1-ts20 | Human | Breast/Mammary | Adherent | yes |
| embryonic stem cells | Mouse | Embryo | Adherent | yes |
| e. histolytica | Amoeba | Other | Suspension | yes |
| ef88 | Mouse | Fibroblast | Adherent | yes |
| el-4 | Mouse | Thymus | Suspension | no |
| ebc-1 | Human | Lung | Adherent | no |
| duck (in vivo) | Duck | Other | Suspension | yes |
| ecv | Human | Endothelial | Adherent | no |
| ecr-293 | Human | Kidney | Adherent | no |
| e14tg2a | Mouse | Embryo | Adherent | no |
| e36 | Hamster | Lung | Adherent | no |
| endothelial cells | Rat | Aorta | Adherent | yes |
| (pulmonary aorta) | | | | |
| endothelial cells (aortic) | Pig | Aorta | Adherent | yes |
| ewing sarcoma coh cells | Human | Bone | Suspension | no |
| f9 | Mouse | Testes | Adherent | no |
| fibroblasts (cardiac) | Rat | Fibroblast | Adherent | yes |
| f442-a | Mouse | Preadiopocyte | Adherent | no |
| es-2 ovarian clear cell | Human | Ovary | Adherent | no |
| adenocarcinoma | | | | |
| fetal neurons | Rat | Brain | Adherent | yes |
| epithelial cells | Human | Epithelial | Adherent | yes |
| (sra01/04) | | | | |
| fibroblasts (embryo) | Rat | Fibroblast | Adherent | yes |
| fgc-4 | Rat | Liver | Adherent | yes |
| fak−/− | Mouse | Embryo | Adherent | yes |
| es-d3 | Mouse | Embryo | Adherent | no |
| epithelial cells (rte) | Rat | Trachael | Adherent | yes |
| foreskin fibroblast | Human | Foreskin | Adherent | no |
| flp-in jurkat | Human | Lymphocyte | Suspension | no |
| flp-in cho | Hamster | Ovary | Adherent | no |
| fibroblasts (neonatal | Human | Skin | Adherent | yes |
| dermal) | | | | |
| flp-in 293 | Human | Kidney | Adherent | no |
| flp-in t-rex 293 | Human | Kidney | Adherent | no |
| flp-in cv-1 | Primate - Non | Kidney | Adherent | no |
| | Human | | | |
| fibroblasts | Chicken | Skin | Adherent | yes |
| fibroblasts (normal) | Human | Fibroblast | Adherent | yes |
| fl5.12 | Mouse | Liver | Suspension | no |
| fm3a | Mouse | Breast/Mammary | Adherent | no |
| fr | Rat | Fibroblast | Adherent | no |
| nalm6 | Human | Other | Suspension | no |

### Gene Expression Perturbation

As described above, in some embodiments, in perturbation test states and combination test states that include perturbation of gene expression, the expression of one or more gene in the cell context is perturbed relative to a corresponding baseline cellular context. In some embodiments, the perturbation is achieved by mutation of the genome of the cellular context, e.g., a human cell line in which a gene has been mutated or deleted. In some embodiments, the mutation is caused by a CRISPR reagent introduced into the cell. In some embodiments, the perturbation includes one or more structural variations (e.g., a documented single nucleotide polymorphism “SNP”, an inversion, a deletion, an insertion, or any combination thereof) of a target gene. In some such embodiments, the one or more documented structural variations are homozygous variations. In some such embodiments, the one or more documented structural variations are heterozygous variations. As an example of a homozygous variation in a diploid genome, in the case of a SNP, both chromosomes contain the same allele for the SNP. As an example of a heterozygous variation in a diploid genome, in the case of the SNP, one chromosome has a first allele for the SNP and the complementary chromosome has a second allele for the SNP, where the first and second allele are different.

In some embodiments, the perturbation of gene expression is caused by the introduction of one or more nucleic acid (e.g., one or more siRNA) that are designed to suppress (e.g., knock-down or knock-out) expression of one or more genes in one or more cell types of the cell context. In some embodiments, the perturbation is caused by introduction of a plurality of nucleic acids (e.g., a plurality of siRNA) that are designed to suppress expression of the same gene in one or more cell types of the cell context. For example, 2, 3, 4, 5, 6, 7, 8, 9, 10, or more siRNA molecules targeting different sequences (e.g., overlapping and/or non-overlapping) of the same gene. In some embodiments, the perturbation is caused by introduction of one or more nucleic acid (e.g., one or more siRNA) that are designed to suppress expression of multiple genes, e.g., 2, 3, 4, 5, 6, 7, 8, 9, 10, or more genes in one or more cell types of the cell context. In some embodiments, the plurality of genes express proteins involved in a common pathway (e.g., a metabolic or signaling pathway) in one or more cell types of the cell context. In some embodiments, the plurality of genes express proteins involved in different pathways in one or more cell types of the cell context. In some embodiments, the different pathways are partially redundant pathways for a particular biological function, e.g., different cell cycle checkpoint pathways. In some embodiments, the perturbation is suppression of a gene known to be associated with a disease (e.g., a checkpoint inhibitor gene associated with a cancer). In some embodiments, the perturbation is suppression of a gene known to be associated with a cellular phenotype (e.g., a gene that causes a metabolic phenotype in cultured cells when suppressed). In some embodiments, the perturbation is suppression of a gene that has not previously been associated with a disease or cellular phenotype.

In some embodiments, a cell context is perturbed by exposure to a small interfering RNA (siRNA), e.g., a double-stranded RNA molecule, 20-25 base pairs in length that interferes with the expression of a specific gene with a complementary nucleotide sequence by degrading mRNA after transcription preventing translation of the gene. An siRNA is an RNA duplex that can reduce gene expression through enzymatic cleavage of a target mRNA mediated by the RNA induced silencing complex (RISC). An siRNA has the ability to inhibit targeted genes with near specificity. See, Agrawal et al., 2003, “RNA interference: biology, mechanism, and applications,” Microbiol Mol Biol Rev. 67: 657-85; and Reynolds et al., 2004, “Rational siRNA design for RNA interference,” Nature Biotechnology 22, 326-330, each of which is hereby incorporated by reference. In some such embodiments, the perturbation is achieved by transfecting the siRNA into the one or more cells, DNA-vector mediated production, or viral-mediated siRNA synthesis. See, for example, Paddison et al., 2002, “Short hairpin RNAs (shRNAs) induce sequence-specific silencing in mammalian cells,” Genes Dev. 16:948-958; Sui et al., 2002, A DNA vector-based RNAi technology to suppress gene expression in mammalian cells,” Proc Natl Acad Sci USA 99:5515-5520; Brummelkamp et al., 2002, “A system for stable expression of short interfering RNAs in mammalian cells,” Science 296:550-553; Paddison et al., 2004, “Short hairpin activated gene silencing in mammalian cells,” Methods Mol Biol 265:85-100; Wong et al. 2003, “CIITAregulated plexin-A1 affects T-cell-dendritic cell interactions, Nat Immunol 2003, 4:891-898; Tomar et al., 2003, “Use of adeno-associated viral vector for delivery of small interfering RNA. Oncogene 22:5712-5715; Rubinson et al., 2003 “A lentivirus-based system to functionally silence genes in primary mammalian cells, stem cells and transgenic mice by RNA interference,” Nat Genet 33:401-406; Moore et al., 2005, “Stable inhibition of hepatitis B virus proteins by small interfering RNA expressed from viral vectors,” J Gene Med; and Tran et al., 2003, “Expressing functional siRNAs in mammalian cells using convergent transcription, BMC Biotechnol 3:21; each of which is hereby incorporated by reference.

In some embodiments, a cell context is perturbed by exposure to a short hairpin RNA (shRNA). See, Taxman et al., 2006, “Criteria for effective design, construction, and gene knockdown by shRNA vectors,” BMC Biotechnology 6:7 (2006), which is hereby incorporated by reference. In some such embodiments, the perturbation is achieved by DNA-vector mediated production, or viral-mediated siRNA synthesis as generally discussed in the references cited above for siRNA.

In some embodiments, a cell context is perturbed by exposure to a single guide RNA (sgRNA) used in the context of palindromic repeat (e.g., CRISPR) technology. See, Sander and Young, 2014, “CRISPR-Cas systems for editing, regulating and targeting genomes,” Nature Biotechnology 32, 347-355, hereby incorporated by reference, in which a catalytically-dead Cas9 (usually denoted as dCas9) protein lacking endonuclease activity to regulate genes in an RNA-guided manner. Targeting specificity is determined by complementary base-pairing of a single guide RNA (sgRNA) to the genomic loci. sgRNA is a chimeric noncoding RNA that can be subdivided into three regions: a 20 nt base-pairing sequence, a 42 nt dCas9-binding hairpin and a 40 nt terminator. In some embodiments, when designing a synthetic sgRNA, only the 20 nt base-pairing sequence is modified from the overall template. In some such embodiments, the perturbation is achieved by DNA-vector mediated production, or viral-mediated sgRNA synthesis.

### Compound Perturbation

As described above, in some embodiments that include compound test states and/or combination test states, the cellular context is exposed to a target compound for which interaction or similarity information, relative to a second biological agent (e.g., a gene, candidate drug, soluble factor, or toxin).

In some embodiments, the compound is a candidate therapeutic agent. In some embodiments, the candidate therapeutic agent is rationally selected, e.g., because of a known property of the molecule. In some embodiments, the candidate therapeutic agent has already been found to have therapeutic benefits, such as a previously approved therapeutic agent or a preclinical/clinical molecule, for which additional information about one or more biological interaction properties are sought. In some embodiments, the candidate therapeutic agent is from a compound library, e.g., where a portion or all of the compounds in the library are being screened for biological interactions. Many commercial and proprietary chemical libraries exist, for example, the Diversity Compound Library (Charles River) contains 689,000 sourced compounds, the EXPRESS-Pick Collection Stock (Chem Bridge) contains over 480,000 chemical compounds, the CORE Library Stock contains more than 690,000 (Chem Bridge) compounds, and pharmaceutical companies have their own proprietary compound libraries having over a million compounds (Macarron R, et al., “Impact of high-throughput screening in biomedical research,” Nat Rev Drug Discov., 10(3):188-95 (2011), which is hereby incorporated by reference). However, the number of possible compounds is nearly limitless. For example, the PubChem database (see, Wang, Y., et al., Nucleic Acids Res. 40:D400-D412 (2012)), a public repository for screening data, lists over 93 million compounds for which screening data has been generated.

In some embodiments, a candidate therapeutic agent is a chemical compound that satisfies the Lipinski rule of five criteria. In some embodiments, a candidate therapeutic agent is an organic compound that satisfies two or more rules, three or more rules, or all four rules of the Lipinski's Rule of Five: (i) not more than five hydrogen bond donors (e.g., OH and NH groups), (ii) not more than ten hydrogen bond acceptors (e.g. N and O), (iii) a molecular weight under 500 Daltons, and (iv) a Log P under 5. The “Rule of Five” is so called because three of the four criteria involve the number five. See, Lipinski, 1997, “Experimental and computational approaches to estimate solubility and permeability in drug discovery and development settings,” Adv. Drug Del. Rev. 23, 3-26, which is hereby incorporated herein by reference in its entirety. In some embodiments, the test perturbation satisfies one or more criteria in addition to Lipinski's Rule of Five. For example, in some embodiments, the test perturbation is a compound with five or fewer aromatic rings, four or fewer aromatic rings, three or fewer aromatic rings, or two or fewer aromatic rings.

In some embodiments, the compound is a soluble factor, e.g., a growth factor, chemokine, cytokine, adhesion molecule, protease, or shed receptor. In some embodiments, the compound is a cytokine or mixture of cytokines. See Heike and Nakahata, 2002, “Ex vivo expansion of hematopoietic stem cells by cytokines,” Biochim Biophys Acta 1592, 313-321, which is hereby incorporated by reference. In some embodiments the compound is a particular type of cytokine, e.g., a lymphokine, a chemokine, an interferon, a tumor necrosis factor, etc. In some embodiments the soluble factor is a lymphokine, e.g., Interleukin 2, Interleukin 3, Interleukin 4, Interleukin 5, Interleukin 6, granulocyte-macrophage colony-stimulating factor, interferon gamma, etc. In some embodiments, the soluble factor is a chemokine, such as a homeostatic chemokine (e.g., CCL14, CCL19, CCL20, CCL21, CCL25, CCL27, CXCL12, CXCL13, etc.) and/or an inflammatory chemokine (e.g., CXCL-8, CCL2, CCL3, CCL4, CCL5, CCL11, CXCL10). In some embodiments, the soluble factor is an interferon (IFN), such as a type I IFN (e.g., IFN-α, IFN-β, IFN-ε, IFN-κ and IFN-ω.), a type II IFN (e.g., IFN-γ), or a type III IFN. In some embodiments, the soluble factor is a tumor necrosis factor, such as TNFα or TNF alpha.

### Cellular Characteristics

Each measurement of a cellular characteristic 113, 115, 117, and 119, used to form the elements of data points 133, 135, 137, and 139, for a corresponding baseline state, perturbation state, compound state, or combination state, respectively, is selected from a plurality of measured cellular characteristics. In some embodiments, the one or more cellular characteristic measurements include one or more of morphological features, expression data, genomic data, epigenomic data, epigenetic data, proteomic data, metabolomics data, toxicity data, bioassay data, etc.

In some embodiments, the corresponding set of elements in each data point 133, 135, 137, and/or 139, includes between 5 test elements and 100,000 test elements. Likewise, in some embodiments, the corresponding set of elements includes a range of elements falling within the larger range discussed above, e.g., from 100 to 100,000, from 1000 to 100,000, from 10,000 to 100,000, from 5 to 10,000, from 100 to 10,000, from 1000 to 10,000, from 5 to 1000, from 100 to 1000, and the like. Generally, the more elements included in the data points, the more information available to identify an interaction between two agents in a biological system. On the other hand, as the number of elements in the set increases, the computational resources required to process the data and manipulate the multidimensional vectors also increases.

In some embodiments, each cellular characteristic is a cellular characteristic that is optically measured, e.g., using fluorescent labels (e.g., cell painting) or using native imaging, as described herein and known to the skilled artisan. In some embodiments, when each cellular characteristic is an optical cellular characteristic, a single image collection step (e.g., that obtains a single image or a series of images at multiple wavebands) can be used to collect image data from multiple samples, e.g., an entire multiwell plate. In some embodiments, a number of images are collected for each well in a multiwell plate. Cellular characteristic extraction is then performed electronically from the collected image(s), limiting the experimental time required to extract cellular characteristics from a large plurality of cell contexts and experimental states.

In some embodiments, a first subset of the cellular characteristics are optically measured (e.g., e.g., using fluorescent labels (e.g., cell painting)), and a second subset of the cellular characteristics are non-optical cellular characteristics. Non-limiting examples of non-optical cellular characteristics include gene expression, protein levels, single endpoint bio-assays, metabolome data, microenvironment data, microbiome data, genome sequence and associated features (e.g., epigenetic data such as methylation, 3D genome structure, chromatin accessibility, etc.), and a relationship and/or change in a particular feature over time, e.g., within a single sample or across a plurality of samples in a time series. Further details about these and other types of non-optical features, as well as collection of data associated with these features, is provided below.

In some embodiments, each cellular characteristic is non-optically measured Non-limiting examples of non-optical cellular characteristics include gene expression, protein levels, single endpoint bio-assays, metabolome data, microenvironment data, microbiome data, genome sequence and associated features (e.g., epigenetic data such as methylation, 3D genome structure, chromatin accessibility, etc.), and a relationship and/or change in a particular feature over time, e.g., within a single sample or across a plurality of samples in a time series. Further details about these and other types of non-optical cellular characteristics, as well as collection of data associated with these cellular characteristics, is provided below. Thus, in some embodiments, multiple assays are performed for each instance (e.g., replicate) of a respective experimental condition, e.g., both a nucleic acid microarray assay and a bioassay are performed from different instances of an experimental condition.

### Optically-Measured Cellular Characteristics

In some embodiments, one or more of the cellular characteristics represent morphological features of a cell, or an enumerated portion of a cell, in the particular experimental condition. Example cellular characteristics include, but are not limited to cell area, cell perimeter, cell aspect ratio, actin content, actin texture, cell solidity, cell extent, cell nuclear area, cell nuclear perimeter, cell nuclear aspect ratio, and algorithm-defined features (e.g., latent features). In some embodiment, example cellular characteristics include, but are not limited to, any of the features found in Table S2 of the reference Gustafsdottir S M, et al., PLoS ONE 8(12): e80999. doi:10.1371/journal.pone.0080999 (2013), which is hereby incorporated by reference.

In some embodiments, such morphological cellular characteristics are measured and acquired using the software program Cellprofiler. See Carpenter et al., 2006, “CellProfiler: image analysis software for identifying and quantifying cell phenotypes,” Genome Biol. 7, R100 PMID: 17076895; Kamentsky et al., 2011, “Improved structure, function, and compatibility for CellProfiler: modular high-throughput image analysis software,” Bioinformatics 2011/doi. PMID: 21349861 PMCID: PMC3072555; and Jones et al., 2008, CellProfiler Analyst: data exploration and analysis software for complex image-based screens, BMC Bioinformatics 9(1):482/doi: 10.1186/1471-2105-9-482. PMID: 19014601 PMCID: PMC261443, each of which is hereby incorporated by reference.

In some embodiments, the measurement of one or more cellular characteristic is a fluorescent microscopy measurement of the cellular characteristic. In some embodiments, one or more optical emitting compounds are used for optical imaging of the cells. In some embodiments, multiple optically distinguishable dyes are used to facilitate measurements of various cellular characteristics, e.g., at least one, two, three, four, five, six, or more optically distinguishable dyes.

Accordingly, in some embodiments, one or more cellular characteristic is measured after exposure of the cell context to the compound and to a panel of fluorescent stains that emit at different wavelengths, such as Concanavalin A/Alexa Fluor 488 conjugate (Invitrogen, cat. no. C11252), Hoechst 33342 (Invitrogen, cat. no. H3570), SYTO 14 green fluorescent nucleic acid stain (Invitrogen, cat. no. S7576), Phalloidin/Alexa Fluor 568 conjugate (Invitrogen, cat. no. A12380), and/or MitoTracker Deep Red (Invitrogen, cat. no. M22426). In some embodiments, measured cellular characteristics include one or more of staining intensities, textural patterns, size, and shape of the labeled cellular structures, as well as correlations between stains across channels, and adjacency relationships between cells and among intracellular structures. In some embodiments, two, three, four, five, six, seven, eight, nine, ten, or more than 10 fluorescent stains, imaged in two, three, four, five, six, seven, or eight channels, are used to measure cellular characteristics including different cellular components and/or compartments.

In some embodiments, one or more cellular characteristics are measured from single cells, groups of cells, and/or a field of view. In some embodiments, cellular characteristics are measured from a compartment or a component (e.g., nucleus, endoplasmic reticulum, nucleoli, cytoplasmic RNA, F-actin cytoskeleton, Golgi, plasma membrane, mitochondria) of a single cell. In some embodiments, each channel includes (i) an excitation wavelength range and (ii) a filter wavelength range in order to capture the emission of a particular dye from among the set of dyes the cell has been exposed to prior to measurement. An example of the dye that is being invoked and the type of cellular component that is measured as a features for five suitable channels is provided in Table 2 below, which is adapted from Table 1 of Bray et al., 2016, “Cell Painting, a high-content image-based assay for morphological profiling using multiplexed fluorescent dyes,” Nature Protocols, 11, p. 1757-74, which is hereby incorporated by reference.

TABLE 2
Example channels used for measuring cellular characteristics

| | | Filter | Filter | Entity |
|---|---|---|---|---|
| | | (excitation; | (emission; | component or |
| Channel | Dye | nm) | nm) | compartment |
| 1 | Hoechst 33342 | 387/11 | 417-477 | Nucleus |
| 2 | Concanavalin A/ | 472/30a | 503-538a | Endoplasmic |
| | Alexa Fluor 488 | | | reticulum |
| | conjugate | | | |
| 3 | SYTO 14 green | 531/40 | 573-613 | Nucleoli, |
| | fluorescent | | | cytoplasmic |
| | nucleic acid | | | RNAb |
| | stain | | | |
| 4 | Phalloidin/ | 562/40 | 622-662c | F-actin |
| | Alexa Fluor | | | cytoskeleton, |
| | 568 conjugate, | | | Golgi, plasma |
| | wheat-germ | | | membrane |
| | agglutinin/ | | | |
| | Alexa Fluor | | | |
| | 555 conjugate | | | |
| 5 | MitoTracker | 628/40 | 672-712 | Mitochondria |
| | Deep Red | | | |

Cell Painting and related variants of cell painting represent another form of imaging technique that holds promise. Cell painting is a morphological profiling assay that multiplexes six fluorescent dyes, imaged in five channels, to reveal eight broadly relevant cellular components or organelles. Cells are plated in multiwell plates, perturbed with the treatments to be tested, stained, fixed, and imaged on a high-throughput microscope. Next, automated image analysis software identifies individual cells and measures any number between one and tens of thousands (but most often approximately 1,000) morphological cellular characteristics (various measures of size, shape, texture, intensity, etc. of various whole-cell and sub-cellular components) to produce a profile that is suitable for the detection of even subtle phenotypes. Profiles of cell populations in different experimental states can be compared to suit many goals, such as identifying the phenotypic impact of chemical or genetic perturbations, grouping compounds and/or genes into functional pathways, and identifying signatures of disease. See, Bray et al., 2016, Nature Protocols 11, 1757-1774.

In some embodiments, the measurement of a cellular characteristic is performed using a label-free imaging technique. Non-invasive, label free imaging techniques have emerged, fulfilling the requirements of minimal cell manipulation for cell based assays in a high content screening context. Among these label free techniques, digital holographic microscopy (Rappaz et al., 2015 Automated multi-parameter measurement of cardiomyocytes dynamics with digital holographic microscopy,” Opt. Express 23, 13333-13347) provides quantitative information that is automated for end-point and time-lapse imaging using 96- and 384-well plates. See, for example, Kuhn, J. 2013, et al., “Label-free cytotoxicity screening assay by digital holographic microscopy,” Assay Drug Dev. Technol. 11, 101-107; Rappaz et al., 2014 “Digital holographic microscopy: a quantitative label-free microscopy technique for phenotypic screening,” Comb. Chem. High Throughput Screen 17, 80-88; and Rappaz et al., 2015 in Label-Free Biosensor Methods in Drug Discovery (ed. Fang, Y.) 307-325, Springer Science+Business Media). Light sheet fluorescence microscopy (LSFM) holds promise for the analysis of large numbers of samples, in 3D high resolution and with fast recording speed and minimal photo-induced cell damage. LSFM has gained increasing popularity in various research areas, including neuroscience, plant and developmental biology, toxicology and drug discovery, although it is not yet adapted to an automated HTS setting. See, Pampaloni et al., 2014, “Tissue-culture light sheet fluorescence microscopy (TC-LSFM) allows long-term imaging of three-dimensional cell cultures under controlled conditions,” Integr. Biol. (Camb.) 6, 988-998; Swoger et al., 2014, “Imaging cellular spheroids with a single (selective) plane illumination microscope,” Cold Spring Harb. Protoc., 106-113; and Pampaloni et al., 2013, “High-resolution deep imaging of live cellular spheroids with light-sheet-based fluorescence microscopy,” Cell Tissue Res. 352, 161-177.

In some embodiments, the measurement of one or more cellular characteristic is performed by a bright field measurement technique. In contrast to measurements obtained by fluorescent microscopy, which requires exposing the cell context to one of more fluorescent stain, bright field microscopy does not require the use of stains, reducing phototoxicity and simplifying imaging setup. Although the lack of stains reduces the contrast provided in bright field images, as compared to fluorescent images, various techniques have been developed to improve cellular imaging in this fashion. For example, Quantitative Phase Microscopy relies on estimation of a phase map generated from images acquired at different focal lengths. See, for example, Curl C L, et al., Cytometry A 65:88-92 (2005), which is incorporated by reference herein. Similarly, a phase map can be measured using lowpass digital filtering, followed by segmentation of individual cells. See, for example, Ali R., et al., Proc. 5th IEEE International Symposium on Biomedical Imaging: From Nano to Macro, ISBI:181-84 (2008), which is incorporated by reference herein. Texture analysis, e.g., where cell contours are extracted after segmentation, can also be used in conjunction with bright field microscopy. See, for example, Korzynska A, et al., Pattern Anal Appl 10:301-19 (2007). Yet other techniques are also available to facilitate use of bright filed microscopy, including z-projection based methods. See, for example, Selinummi J., et al., PLoS One, 4(10):e7497 (2009).

In some embodiments, the measurement of one or more cellular characteristic is performed by a phase contrast measurement technique. Images obtained by phase contrast or differential interference contrast (DIC) microscopy can be digitally reconstructed and quantified. See Koos, 2015, “DIC image reconstruction using an energy minimization framework to visualize optical path length distribution,” Sci. Rep. 6, 30420.

Although particular imaging techniques are specifically described herein, the methods provided herein can be performed using features measured from any of a number of microscopic modalities.

In some embodiments, each cellular characteristic represents a color, texture, or size of the cell context, or an enumerated portion of the cell context. Example features include, but are not limited to cell area, cell perimeter, cell aspect ratio, actin content, actin texture, cell solidity, cell extent, cell nuclear area, cell nuclear perimeter, and cell nuclear aspect ratio. In some embodiment, example features include, but are not limited to, any of the features found in Table S2 of the reference Gustafsdottir S M, et al., PLoS ONE 8(12): e80999. doi:10.1371/journal.pone.0080999 (2013), which is hereby incorporated by reference.

In some embodiments, one or more of the measured cellular characteristics are latent features, e.g., extracted from an image of the cell context. In one embodiment, each respective instance of an experimental state is imaged to form a corresponding two-dimensional pixelated image having a corresponding plurality of native pixel values, and one or more cellular characteristics are generated as a result of a convolution, or a series convolutions, and pooling operators run against native pixel values in the plurality of native pixel values of the corresponding two-dimensional pixelated image. While this is an example of a latent cellular characteristic that can be derived from an image, other latent cellular characteristics and mathematical combinations of latent cellular characteristics can also be used. A non-limiting example of the use of latent cellular characteristics in image-based profiling of cellular structure is found in Ljosa, V., et al., J Biomol. Screen., 18(10):10.1177/1087057113503553 (2013), which is incorporated herein by reference.

### Non-Optically-Measured Cellular Characteristics

In some embodiments one or more of the measured cellular characteristics include expression data, e.g., obtained using a whole transcriptome shotgun sequencing (RNA-Seq) assay that quantifies gene expression from cells (e.g., a single cell) in counts of transcript reads mapped to gene constructs. As such, in some embodiments, RNA-Seq experiments aim at reconstructing all full-length mRNA transcripts concurrently from millions of short reads. RNA-Seq facilitates the ability to look at alternative gene spliced transcripts, post-transcriptional modifications, gene fusion, mutations/SNPs and changes in gene expression over time, or differences in gene expression in different groups or treatments. See, for example, Maher et al., 2009, “Transcriptome sequencing to detect gene fusions in cancer,” Nature. 458 (7234): 97-101, which is hereby incorporated by reference. In addition to mRNA transcripts, RNA-Seq can evaluate and quantify individual members of different populations of RNA including total RNA, mRNA, miRNA, IncRNA, snoRNA, or tRNA within entities. As such, in some embodiments, one or more of the cellular characteristics that is measured is an individual amount of a specific RNA species as determined using RNA-Seq techniques. In some embodiments, RNA-Seq experiments produce counts of component (e.g., digital counts of mRNA reads) that are affected by both biological and technical variation. In some embodiments RNA-Seq assembly is performed using the techniques disclosed in Li et al., 2008, “IsoLasso: A LASSO Regression Approach to RNA-Seq Based Transcriptome Assembly,” Cell 133, 523-536 which is hereby incorporated by reference.

In some embodiments one or more of the measured cellular characteristics are obtained using transcriptional profiling methods such an L1000 panel that measures a set of informative transcripts. In such an approach, ligation-mediated amplification (LMA) followed by capture of the amplification products on fluorescently addressed microspheres beads is extended to a multiplex reaction (e.g., a 1000-plex reaction). For instance, cells growing in 384-well plates are lysed and mRNA transcripts are captured on oligo-dT-coated plates. cDNAs are synthesized from captured transcripts and subjected to LMA using locus-specific oligonucleotides harboring a unique 24-mer barcode sequence and a 5′ biotin label. The biotinylated LMA products are detected by hybridization to polystyrene microspheres (beads) of distinct fluorescent color, each coupled to an oligonucleotide complementary to a barcode, and then stained with streptavidin-phycoerythrin. In this way, each bead can be analyzed both for its color (denoting landmark identity) and fluorescence intensity of the phycoerythrin signal (denoting landmark abundance). See Subramanian et al., “A Next Generation Connectivity Map: L1000 Platform and the First 1,000,000 Profiles,” Cell 171(6), 1437, which is hereby incorporated by reference. In some embodiments, between 500 and 1500 different informative transcripts are measured using this assay.

In some embodiments one or more of the measured cellular characteristics are obtained using microarrays. A microarray (also termed a DNA chip or biochip) is a collection of microscopic nucleic acid spots attached to a solid surface that can be used to measure the expression levels of large numbers of genes simultaneously. Each nucleic acid spot contains picomoles of a specific nucleic acid sequence, known as probes (or reporters or oligos). These can be a short section of a gene or other nucleic acid element that are used to hybridize a cDNA or cRNA (also called anti-sense RNA) sample (called target) under high-stringency conditions. For instance, by way of a non-limiting example, in some embodiments, the microarrays such as the Affymetrix GeneChip microarray, a high density oligonucleotide gene expression array, is used. Each gene on an Affymetrix microarray GeneChip is typically represented by a probe set consisting of 11 different pairs of 25-bp oligos covering features of the transcribed region of that gene. Each pair consists of a perfect match (PM) and a mismatch (MM) oligonucleotide. The PM probe exactly matches the sequence of a particular standard genotype, often one parent of a cross, while the MM differs in a single substitution in the central, 13^{th} base. The MM probe is designed to distinguish noise caused by non-specific hybridization from the specific hybridization signal. See, Jiang, 2008, “Methods for evaluating gene expression from Affymetrix microarray datasets,” BMC Bioinformatics 9, 284, which is hereby incorporated by reference.

In some embodiments one or more of the measured cellular characteristics are obtained using ChIP-Seq data. See, for example, Quigley and Kintner, 2017, “Rfx2 Stabilizes Foxj1 Binding at Chromatin Loops to Enable Multiciliated Cell Gene Expression,” PLoS Genet 13, e1006538, which is hereby incorporated by reference. In some embodiments, ChIP-seq is used to determine how transcription factors and other chromatin-associated proteins influence phenotype-affecting mechanisms in entities (e.g., cells). Specific DNA sites in direct physical interaction with transcription factors and other proteins can be isolated by chromatin immunoprecipitation. ChIP produces a library of target DNA sites bound to a protein of interest (component) in vivo. Parallel sequence analyses are then used in conjunction with whole-genome sequence databases to analyze the interaction pattern of any protein with DNA (Johnson et al., 2007, “Genome-wide mapping of in vivo protein—DNA interactions,” Science. 316: 1497-1502, which is hereby incorporated by reference) or the pattern of any epigenetic chromatin modifications. This can be applied to the set of ChIP-able proteins and modifications, such as transcription factors, polymerases and transcriptional machinery, structural proteins, protein modifications, and DNA modifications.

ChIP selectively enriches for DNA sequences bound by a particular protein (component) in living cells (entities). The ChIP process enriches specific cross-linked DNA-protein complexes using an antibody against the protein (component) of interest. Oligonucleotide adaptors are then added to the small stretches of DNA that were bound to the protein of interest to enable massively parallel sequencing. After size selection, all the resulting ChIP-DNA fragments are sequenced concurrently using a genome sequencer. A single sequencing run can scan for genome-wide associations with high resolution, meaning that features can be located precisely on the chromosomes. Various sequencing methods can be used. In some embodiments the sequences are analyzed using cluster amplification of adapter-ligated ChIP DNA fragments on a solid flow cell substrate to create clusters of clonal copies. The resulting high density array of template clusters on the flow cell surface is sequenced by a Genome analyzing program. Each template cluster undergoes sequencing-by-synthesis in parallel using fluorescently labelled reversible terminator nucleotides. Templates are sequenced base-by-base during each read. Then, the data collection and analysis software aligns sample sequences to a known genomic sequence to identify the ChIP-DNA fragments.

In some embodiments one or more of the measured cellular characteristics are obtained using ATAC-seq (Assay for Transposase-Accessible Chromatin using sequencing), which is a technique used in molecular biology to study chromatin accessibility. See Buenrostro et al., 2013, “Transposition of native chromatin for fast and sensitive epigenomic profiling of open chromatin, DNA-binding proteins and nucleosome position,” Nature Methods 10, 1213-1218, which is hereby incorporated by reference. In some embodiments, ATAC-seq make use of the action of the transposase Tn5 on the genomic DNA of an entity. See, for example, Buenrostro et al., 2015, “ATAC-seq: A Method for Assaying Chromatin Accessibility Genome-Wide,” Current Protocols in Molecular Biology: 21.29.1-21.29.9, which is hereby incorporated by reference. Transposases are enzymes catalyzing the movement of transposons to other parts in the genome. While naturally occurring transposases have a low level of activity, ATAC-seq employs a mutated hyperactive transposase. The high activity allows for highly efficient cutting of exposed DNA and simultaneous ligation of specific sequences, called adapters. Adapter-ligated DNA fragments are then isolated, amplified by PCR and used for next generation sequencing. See Buenrostro et al., 2013, “Transposition of native chromatin for fast and sensitive epigenomic profiling of open chromatin, DNA-binding proteins and nucleosome position,” Nature Methods 10, 1213-1218, which is hereby incorporated by reference.

While not intending to be limited to any particular theory, transposons are believed to incorporate preferentially into genomic regions free of nucleosomes (nucleosome-free regions) or stretches of exposed DNA in general. Thus enrichment of sequences from certain loci in the genome indicates absence of DNA-binding proteins or nucleosome in the region. An ATAC-seq experiment will typically produce millions of next generation sequencing reads that can be successfully mapped on the reference genome. After elimination of duplicates, each sequencing read points to a position on the genome where one transposition (or cutting) event took place during the experiment. One can then assign a cut count for each genomic position and create a signal with base-pair resolution. This signal is used as a features in some embodiments of the present disclosure. Regions of the genome where DNA was accessible during the experiment will contain significantly more sequencing reads (since that is where the transposase preferentially acts), and form peaks in the ATAC-seq signal that are detectable with peak calling tools. In some embodiments, such peaks, and their locations in the genome are used as features. In some embodiments, these regions are further categorized into the various regulatory element types (e.g., promoters, enhancers, insulators, etc.) by integrating further genomic and epigenomic data such as information about histone modifications or evidence for active transcription. Inside the regions where the ATAC-seq signal is enriched, one can also observe sub-regions with depleted signal. These sub-regions, typically only a few base pairs long, are considered to be “footprints” of DNA-binding proteins. In some embodiments, such footprints, or their absence or presence thereof are used as cellular characteristics.

In some embodiments flow cytometry methods using Luminex beads, are used to obtain values for one or more of the measured cellular characteristics. See for example, Süsal et al., 2013, Transfus Med Hemother 40, 190-195, which is hereby incorporated by reference. For instance, the Luminex-supported single antigen bead (L-SAB) test allows for the characterization of human leukocyte antigen (HLA) antibody specificities. In such a flow cytometric method, microbeads coated with recombinant single antigen HLA molecules are employed in order to differentiate antibody reactivity in two reaction tubes against 100 different HLA class I and 100 different HLA class II alleles. An approximation of the strength of antibody reactivity is derived from the mean fluorescence intensity (MFI) and in some embodiments this serves as features in the present disclosure. In addition to antibody reactivity against HLA-A, -B, -C, -DR and -DQB antigens, L-SAB is capable of detecting antibodies against HLA-DQA, -DPA, and -DPB antigens. In some embodiments, other Luminex kits are used for detection of non-HLA antibodies in order to derive values for one or more features for entities in accordance with the present disclosure. For instance, in some embodiments, major histocompatibility complex class I-related chain A (MICA) and human neutrophil antibodies, and kits that utilize, instead of recombinant HLA molecules, affinity purified pooled human HLA molecules obtained from multiple cell lines (screening test to detect presence of HLA antibodies without further specification) or phenotype panels in which each bead population bears either HLA class I or HLA class II proteins of a cell lines derived from a single individual (panel reactivity, PRA-test) are used to determine values for cellular characteristics in accordance with an embodiment of the present disclosure.

In some embodiments, flow cytometry methods, such fluorescent cell barcoding, is used to obtain values for one or more of the measured cellular characteristics. Fluorescent cell barcoding (FCB) enables high throughput, e.g. high content flow cytometry by multiplexing samples of entities prior to staining and acquisition on the cytometer. Individual cell samples (entities) are barcoded, or labeled, with unique signatures of fluorescent dyes so that they can be mixed together, stained, and analyzed as a single sample. By mixing samples prior to staining, antibody consumption is typically reduced 10 to 100-fold. In addition, data robustness is increased through the combination of control and treated samples, which minimizes pipetting error, staining variation, and the need for normalization. Finally, speed of acquisition is enhanced, enabling large profiling experiments to be run with standard cytometer hardware. See, for example, Krutzik, 2011, “Fluorescent Cell Barcoding for Multiplex Flow Cytometry,” Curr Protoc Cytom Chapter 6: Unit 6.31, which is hereby incorporated by reference.

In some embodiments, metabolomics is used to obtain values for one or more of the cellular characteristics. Metabolomics is a systematic evaluation of small molecules in order to obtain biochemical insight into disease pathways. In some embodiments, such metabolomics comprises evaluation of plasma metabolomics in diabetes (Newgard et al., 2009, “A branched-chain amino acid-related metabolic signature that differentiates obese and lean humans and contributes to insulin resistance,” Cell Metab 9: 311-326, 2009) and ESRD (Wang, 2011, “RE: Metabolite profiles and the risk of developing diabetes,” Nat Med 17: 448-453). In some embodiments, urine metabolomics is used to obtain values for one or more of the features. Urine metabolomics offers a wider range of measurable metabolites because the kidney is responsible for concentrating a variety of metabolites and excreting them in the urine. In addition, urine metabolomics may offer direct insights into biochemical pathways linked to kidney dysfunction. See, for example, Sharma, 2013, “Metabolomics Reveals Signature of Mitochondrial Dysfunction in Diabetic Kidney Disease,” J Am Soc Nephrol 24, 1901-12, which is hereby incorporated by reference.

In some embodiments, mass spectrometry is used to obtain values for one or more of the measured cellular characteristics. For instance, in some embodiments, protein mass spectrometry is used to obtain values for one or more of the measured cellular characteristics. In particular, in some embodiments, biochemical fractionation of native macromolecular assemblies within entities followed by tandem mass spectrometry is used to obtain values for one or more of the measured cellular characteristics. See, for example, Wan et al., 2015, “Panorama of ancient metazoan macromolecular complexes,” Nature 525, 339-344, which is hereby incorporated by reference. Tandem mass spectrometry, also known as MS/MS or MS2, involves multiple steps of mass spectrometry selection, with some form of fragmentation occurring in between the stages. In a tandem mass spectrometer, ions are formed in the ion source and separated by mass-to-charge ratio in the first stage of mass spectrometry (MS1). Ions of a particular mass-to-charge ratio (precursor ions) are selected and fragment ions (product ions) are created by collision-induced dissociation, ion-molecule reaction, photodissociation, or other process. The resulting ions are then separated and detected in a second stage of mass spectrometry (MS2). In some embodiments the detection and/or presence of such ions serve as the one or more of the measured cellular characteristics.

In some embodiments, the cellular characteristics that are observed for an experimental state are post-translational modifications that modulate activity of proteins within a cell. In some such embodiments, mass spectrometric peptide sequencing and analysis technologies are used to detect and identify such post-translational modifications. In some embodiments, isotope labeling strategies in combination with mass spectrometry are used to study the dynamics of modifications and this serves as a measured feature. See for example, Mann and Jensen, 2003 “Proteomic analysis of post-translational modifications,” Nature Biotechnology 21, 255-261, which is hereby incorporated by reference. In some embodiments, mass spectrometry is user to determine splice variants in experimental states, for instance, splice variants of components within experimental states, and such splice variants and the detection of such splice variants serve as measured cellular characteristics. See for example, Nilsen and Graveley, 2010, “Expansion of the eukaryotic proteome by alternative splicing, 2010, Nature 463, 457-463, which is hereby incorporated by reference.

In some embodiments, imaging cytometry is used to obtain values for one or more of the measured cellular characteristics. Imaging flow cytometry combines the statistical power and fluorescence sensitivity of standard flow cytometry with the spatial resolution and quantitative morphology of digital microscopy. See, for example, Basiji et al., 2007, “Cellular Image Analysis and Imaging by Flow Cytometry,” Clinics in Laboratory Medicine 27, 653-670, which is hereby incorporated by reference.

In some embodiments, electrophysiology is used to obtain values for one or more of the measured cellular characteristics. See, for example, Dunlop et al., 2008, “High-throughput electrophysiology: an emerging paradigm for ion-channel screening and physiology,” Nature Reviews Drug Discovery 7, 358-368, which is hereby incorporated by reference.

In some embodiments, proteomic imaging/3D imaging is used to obtain values for one or more of the measured cellular characteristics. See for example, United States Patent Publication No. 20170276686 A1, entitled “Single Molecule Peptide Sequencing,” which is hereby incorporated by reference. Such methods can be used to large-scale sequencing of single peptides in a mixture from an entity, or a plurality of entities at the single molecule level.

### Assay Parameters

As described herein with reference to FIG. 3, in some embodiments, each cellular characteristics measurement is obtained in replicate, e.g., each experimental condition representative of an experimental state (e.g., a baseline state, perturbation state, compound state, and/or combination state) is performed more than once and each cellular characteristic measurement is obtained from each instance of the condition. In some embodiments, cellular characteristics measurements are obtained from at least 2, 3, 4, 5, 6, 7, 8, 9, 10, 15, 20, 25, 50, 75, 100, 500, or more instances of every condition, e.g., experimental conditions are prepared in two or more replicates.

With respect to the concentrations of compounds used for any particular experimental condition representative of a compound state or combination state, the skilled artisan will know how to select a concentration for a given compound, e.g., based upon one or more known or expected property of the compound such as molecular weight, solubility, presence or particular functional groups, known or expected interactions, known or expected toxicity, etc. For example, in some embodiments, where a respective compound is known to be toxic to a particular cell context, the concentration of the compound may be adjusted, e.g., relative to the concentration used for other compounds. Generally, the time over which a cell context is exposed to a compound is influenced by the particular cellular characteristics being measured and/or the particular assay from which the cellular characteristic data is being generated. For example, where the assay being used measures a phenomenon that occurs rapidly following exposure of the cell context to the compound, the cell context does not need to be exposed to the compound for a long period of time prior to measurement of the feature. Conversely, where the assay being used measures a phenomenon that occurs slowly, or after a significant delay, following exposure of the cell context to the compound, a longer incubation time should be used prior to measuring the feature.

In some embodiments, e.g., where latent features are being extracted from a cell context, the time over which the cell context is exposed to a compound prior to measurement is determined stochastically. In some embodiments, the time over which the cell context is exposed to a compound prior to measurement is determined based on experience or trial and error with a particular assay or phenomenon. In one embodiment, exposure of the amount of the respective compound to the cell context is for at least one hour prior to obtaining the measurement. In some embodiments, the measurement is obtained by cellular imaging, e.g., using fluorescent labels (e.g., cell painting) or using native imaging, as described herein and known to the skilled artisan. In some embodiments, exposure of the amount of the respective compound to the cell context is for at least one hour prior to obtaining an image.

In some embodiments cellular characteristic data is acquired using an automated cellular imaging system (e.g., ImageXpress Micro, Molecular Devices), where cell contexts have been arranged in multiwell plates (e.g., 384-well plates) after they have been stained with a panel of dyes that emit at different discrete wavelengths (e.g., Hoechst 33342, Alexa Fluor 594 phalloidin, etc.). In some embodiments the cell contexts are imaged with an exposure that is a determined by the marker dye used (e.g., 15 ms for Hoechst, 1000 ms for phalloidin), at 20× magnification with 2× binning. For each well, in some embodiments the optimal focus is found using laser auto-focusing on a particular dye channel (e.g., the Hoechst channel). In some embodiments the automated microscope is then programmed to collect a z-stack of 32 images (z=0 at the optimal focal plane, 16 images above the focal plane, 16 below) with 2 μm between slices. In some embodiments each well contains several thousand cells in them, and thus each digital representation of a well captured by a camera represents several thousand cells in each of several different wells. In some embodiments, segmentation software is used to identify individual cells in the digital images and moreover various components (e.g., cellular components) within individual cells. Once the cellular components are segmented and identified, mathematical transformations are performed on these components on order to obtain the measurements of features.

### Dimensional Reduction

Featurization of the cellular characteristics is performed by a dimensional reduction technique that uses a statistical feature selection or feature extraction procedure known in the art, for example, principal component analysis, non-negative matrix factorization, kernel PCA, graph-based kernel PCA, linear discriminant analysis, generalized discriminant analysis, and use of an autoencoder. This, in turn, reduces the computational burden of analyzing the data set by compressing the data in order to make the method more computationally efficient, e.g., by allowing the computer to apply an algorithm to the smaller dataset rather than the full dataset.

Principle component analysis (PCA) reduces the dimensionality of a multidimensional data point (e.g., baseline state vectors 232, perturbation state vectors 234, compound state vectors 236, and/or combination state vectors 238) by transforming the plurality of elements (e.g., the elements shown for data points 133, 135, 137, 139 in FIG. 3D) to a new set of variables (principal components) that summarize the features of a training set. See, for example, Jolliffe, 1986, Principal Component Analysis, Springer, New York, which is hereby incorporated by reference. PCA is also described in Draghici, 2003, Data Analysis Tools for DNA Microarrays, Chapman & Hall/CRC, which is hereby incorporated by reference. Principal components (PCs) are uncorrelated and are ordered such that the kth PC has the kth largest variance among PCs across the observed data for the features. The kth PC can be interpreted as the direction that maximizes the variation of the projections of the data points such that it is orthogonal to the first k−1 PCs. The first few PCs capture most of the variation in the observed data. In contrast, the last few PCs are often assumed to capture only the residual “noise” in the observed data. As such, the principal components derived from PCA can serve as the basis of vectors that are used in accordance with the present disclosure.

Non-negative matrix factorization and non-negative matrix approximation reduce the dimensionality of a multidimensional matrix by factoring the matrix into two matrices, each of which have significantly lower dimensionality, but which provide a product having the same, or approximately the same, dimensionality as the original higher-dimensional matrix. See, for example, Lee and Seung, “Learning the parts of objects by non-negative matrix factorization, Nature, 401(6755):788-91 (1999), which is hereby incorporated by reference. See also Dhillon and Sra, “Generalized Nonnegative Matrix Approximations with Bregman Divergences,” Advances in Neural Information Processing Systems 18 (NIPS 2005), which is hereby incorporated by reference.

Kernel PCA is an extension of PCA in which N elements of a vector are mapped onto a N-dimensional space using a non-trivial, arbitrary function, creating projections of the elements onto principle components lying on a lower dimensional subspace. In this fashion, kernel PCA is better equipped than PCA to reduce the dimensionality of non-linear data. See, for example, Scholkopf, “Nonlinear Component Analysis as a Kernel Eigenvalue Problem,” Neural Computation, 10: 1299-1319 (198), which is hereby incorporated by reference.

Linear discriminant analysis (LDA), like PCA, reduces the dimensionality of a multidimensional vector by transforming the plurality of elements (e.g., measured elements 216) to a new set of variables (principal components) that summarize the features of the training set. However, unlike PCA, LDA is a supervised feature extraction method which (i) calculates between-class variance, (ii) calculates within-class variance, and then (iii) constructs a lower dimensional-representation that maximizes between-class variance and minimizes within-class variance. See, for example, Tharwat, A., et al., “Linear discriminant analysis: A detailed tutorial,” AI Communications, 30:169-90 (2017), which is hereby incorporated by reference.

Generalized discriminant analysis (GDA), similar to kernel PCA, maps non-linear input elements of multidimensional vectors into higher-dimensional space to provide linear properties of the elements, which can then be analyzed according to classical linear discriminant analysis. In this fashion, GDA is better equipped than LDA to reduce the dimensionality of non-linear data. See, for example, Baudat and Anouar, “Generalized Discriminant Analysis Using a Kernel Approach,” Neural Comput., 12(10):2385-404 (2000).

Autoencoders are artificial neural networks used to learn efficient data codings in an unsupervised learning algorithm that applies backpropagation. Autoencoders consist of two parts, an encoder and a decoder. The encoder reads an input vector and compress it to a lower-dimensional vector, and the decoder reads the compressed vector and recreates the input vector. See, for example, Chapter 14 of Goodfellow et al., “Deep Learning,” MIT Press (2016); Hinton and Salakhutdinov, Science, 313(5786):504-07 (2006), both of which are is hereby incorporated by reference.

In some embodiments, the featurized data terms account for at least ninety percent of the variance of the plurality of cellular characteristics measured across the experimental states. For example, in some embodiments, the featurized data terms are pruned to provide filtered featurized data terms, containing the featurized data terms that account for the greatest variance in the training set, e.g., at least 90%, 95%, 99%, 99.9%, 99.99%, or more variance.

Yet other dimension reductions techniques known in the art may also be applied to the methods described herein. For example, in some embodiments, a subset of measured features is selected for inclusion in a reduced dimension representation of a data point, while discarding other features, e.g., based on optimality criterion in linear regression. See, for example, Draper and Smith, “Applied Regression Analysis,” 2d Edition, New York: John Wiley & Sons, Inc. (1981), which is hereby incorporated by reference. Similarly, in some embodiments, discrete methods, in which features are either selected or discarded, e.g., a leaps and bounds procedure, are used. See, for example, Furnival and Wilson, “Regressions by Leaps and Bounds,” Technometrics, 16(4):499-511 (1974), which is hereby incorporated by reference. Likewise, in some embodiments, linear regression by forward selection, backward elimination, or bidirectionsl elimination are used. See, for example, Draper and Smith, “Applied Regression Analysis,” 2d Edition, New York: John Wiley & Sons, Inc. (1981). In yet other embodiments, shrinkage methods, e.g., methods that reduce/shrink the redundant or irrelevant features in a more continuous fashion are used, e.g., ridge regression, Lasso, and Derived Input Direction Methods (e.g., PCR, PLS).

### Example 1—Identification of Gene-Drug Interactions Using Phenomic Data

In order to determine whether gene-drug interactions between could be identified based on phenomic data, a pilot experiment was performed to test whether a significant interaction could be identified between the VEGF gene and a VEGF inhibitor, Ki8751. As a negative control, a second experiment was performed to test whether a significant interaction between the VEGF gene and a JAK inhibitor, ruxolitinib, could be identified in the same fashion.

Briefly, cellular characteristic data from a plurality of different instances of each of a baseline state (mammalian cells; no siRNA; no inhibitor), a perturbation state (mammalian cells; anti-VEGF siRNA; no inhibitor), a first drug state (mammalian cells; no siRNA; Ki8751), a second drug state (mammalian cells; no siRNA; ruxolitinib), a first combination state (mammalian cells; anti-VEGF siRNA; Ki8751), and a second combination state (mammalian cells; anti-VEGF siRNA; ruxolitinib) were acquired using a modified version the cellular staining and cellular characteristic detection method described in Bray Mass., et al., Nat. Protoc., 11(9):1757-74 (2016), generating measurements for over 1000 different cellular characteristics for each experimental state. The data was normalized and then featurized by principal component analysis.

Two-way ANOVA on an ordinary least squares linear model, a formal statistical test of whether there's an interaction between the drug and the cytokine gene for a given principal component, was performed on the first principal component of the baseline state, the perturbation state (anti-VEGF siRNA), the first drug state (VEGF inhibitor), and the first combination state (anti-VEGF siRNA and VEGF inhibitor). As shown in the Table 3 below, a statistically significant interaction between the anti-VEGF siRNA and the VEGF inhibitor was detected (p=0.008).

TABLE 3
Statistics for two-way ANOVA of principal component P0, for
an interaction between an anti-VEGF siRNA and a VEGF inhibitor.
PR(>F) is the p-value for each respective interaction.

| | sum_sq | df | F | PR(>F) |
|---|---|---|---|---|
| Intercept | 602.325263 | 1.0 | 16.310766 | 0.000060 |
| siRNA | 1.670956 | 1.0 | 0.045249 | 0.831607 |
| drug | 700.375597 | 1.0 | 13.965937 | 0.000015 |
| siRNA:drug | 257.941062 | 1.0 | 6.984958 | 0.008399 |
| Residual | 26477.432314 | 717.0 | NaN | NaN |

Similarly, two-way ANOVA of principal components P1, P2, and P3 were also performed, as reported in tables 4, 5, and 6, respectively.

TABLE 4
Statistics for two-way ANOVA of principal component P1, for
an interaction between an anti-VEGF siRNA and a VEGF inhibitor.
PR(>F) is the p-value for each respective interaction.

| | df | sum_sq | mean_sq | F | PR(>F) |
|---|---|---|---|---|---|
| siRNA | 1.0 | 74.634652 | 74.634652 | 0.800671 | 0.371193 |
| drug | 1.0 | 1.745488 | 1.745488 | 0.018725 | 0.891195 |
| siRNA:drug | 1.0 | 0.003549 | 0.003549 | 0.000038 | 0.995078 |
| Residual | 717.0 | 66835.274634 | 93.215167 | NaN | NaN |

TABLE 5
Statistics for two-way ANOVA of principal component P2, for
an interaction between an anti-VEGF siRNA and a VEGF inhibitor.
PR(>F) is the p-value for each respective interaction.

| | df | sum_sq | mean_sq | F | PR(>F) |
|---|---|---|---|---|---|
| siRNA | 1.0 | 377.527619 | 377.527619 | 3.584736 | 0.058715 |
| drug | 1.0 | 35.597917 | 35.597917 | 0.338013 | 0.561161 |
| siRNA: | 1.0 | 37.585425 | 37.585425 | 0.356885 | 0.550430 |
| drug | | | | | |
| Residual | 717.0 | 75511.092180 | 105.315331 | NaN | NaN |

TABLE 6
Statistics for two-way ANOVA of principal component P3, for
an interaction between an anti-VEGF siRNA and a VEGF inhibitor.
PR(>F) is the p-value for each respective interaction.

| | df | sum_sq | mean_sq | F | PR(>F) |
|---|---|---|---|---|---|
| siRNA | 1.0 | 0.435937 | 0.435937 | 0.014140 | 0.905378 |
| drug | 1.0 | 31.933716 | 31.933716 | 1.035820 | 0.309139 |
| siRNA: | 1.0 | 173.671661 | 173.671661 | 5.633312 | 0.017885 |
| drug | | | | | |
| Residual | 717.0 | 22104.683734 | 30.829405 | NaN | NaN |

As shown in the tables above, a significant interaction between the anti-VEGF siRNA and VEGF inhibitor was also identified for principal component P3 (p=0.018), but not for principal components P1 (p=0.995) and P2 (p=0.550).

Two-way ANOVA were then performed against principal components P0, P1, P2, and P3 of the baseline state, the perturbation state (anti-VEGF siRNA), the second drug state (JAK inhibitor), and the second combination state (anti-VEGF siRNA and JAK inhibitor). As shown in Tables 7-10 below, no statistically significant interaction between the anti-VEGF siRNA and the JAK inhibitor was detected for any of principal components P0, P1, P2, or P3 (p=0.008).

TABLE 7
Statistics for two-way ANOVA of principal component P0, for
an interaction between an anti-VEGF siRNA and a JAK inhibitor.
PR(>F) is the p-value for each respective interaction.

| | df | sum_sq | mean_sq | F | PR(>F) |
|---|---|---|---|---|---|
| siRNA | 1.0 | 1.616575 | 1.616575 | 0.047567 | 0.827414 |
| drug | 1.0 | 8.691292 | 8.691292 | 0.255737 | 0.613219 |
| siRNA:drug | 1.0 | 7.675465 | 7.675465 | 0.225847 | 0.634765 |
| Residual | 718.0 | 24401.402698 | 33.985241 | NaN | NsN |

TABLE 8
Statistics for two-way ANOVA of principal component P1, for
an interaction between an anti-VEGF siRNA and a JAK inhibitor.
PR(>F) is the p-value for each respective interaction.

| | df | sum_sq | mean_sq | F | PR(>F) |
|---|---|---|---|---|---|
| siRNA | 1.0 | 16.839348 | 16.839348 | 0.176992 | 0.674097 |
| drug | 1.0 | 223.820521 | 223.820521 | 2.352495 | 0.125523 |
| siRNA: | 1.0 | 0.236537 | 0.236537 | 0.002486 | 0.960247 |
| drug | | | | | |
| Residual | 718.0 | 68311.794521 | 95.141775 | NaN | NaN |

TABLE 9
Statistics for two-way ANOVA of principal component P2, for
an interaction between an anti-VEGF siRNA and a JAK inhibitor.
PR(>F) is the p-value for each respective interaction.

| | df | sum_sq | mean_sq | F | PR(>F) |
|---|---|---|---|---|---|
| siRNA | 1.0 | 291.785955 | 291.785955 | 2.770491 | 0.096453 |
| drug | 1.0 | 13.596478 | 13.596478 | 0.129098 | 0.719475 |
| siRNA: | 1.0 | 0.072536 | 0.072536 | 0.000689 | 0.979070 |
| drug | | | | | |
| Residual | 718.0 | 75619.198798 | 105.319218 | NaN | NaN |

TABLE 10
Statistics for two-way ANOVA of principal component P3, for
an interaction between an anti-VEGF siRNA and a JAK inhibitor.
PR(>F) is the p-value for each respective interaction.

| | df | sum_sq | mean_sq | F | PR(>F) |
|---|---|---|---|---|---|
| siRNA | 1.0 | 37.185829 | 37.185829 | 1.213102 | 0.271088 |
| drug | 1.0 | 21.499934 | 21.499934 | 0.701386 | 0.402597 |
| siRNA:drug | 1.0 | 7.333086 | 7.333086 | 0.239225 | 0.624916 |
| Residual | 718.0 | 22009.220307 | 30.653510 | NaN | NaN |

These results show that a fairly standard statistical test for interaction between two categorical variables (two-way ANOVA) reveals nice interactions for a few principal components derived from cellular characteristic measurements in a positive control (VEGF and VEGF inhibitor). These results also show that the two-way ANOVA test do not show an interaction between VEGF and a JAK inhibitor, serving as a negative control. Thus, these results are proof of principal that the assay described herein can identify biologically significant interactions between biological agents, e.g., a gene and a drug acting through the gene.

### Example 2—Identification of Gene-Drug Interactions Using Phenomic Screening Data

In order to further test whether gene-drug interactions between could be identified based on large data sets of phenomic screening, a second experiment was performed to look for interactions between a plurality of compounds and perturbations in the IL6 and IL13 gene. The plurality of compounds included a first sub-plurality of compounds that are known JAK inhibitors. Since IL6 and IL13 act through various JAK receptors in vivo, the hypothesis is that the JAK inhibitors in the plurality of compounds are more likely to show an interaction with the IL6 and IL13 perturbations.

Briefly, cellular characteristic data from a plurality of different instances of each of a baseline state (mammalian cells; no siRNA; no compound), an IL6 perturbation state (mammalian cells; anti-IL6 siRNA; no compound), an IL13 perturbation state (mammalian cells; anti-IL13 siRNA; no compound), a plurality of compound states (mammalian cells; no siRNA; compound), a plurality of IL6 combination states (mammalian cells; anti-IL6 siRNA; compound), and a plurality of IL13 combination states (mammalian cells; anti-IL13 siRNA; compound) were acquired using a modified version the cellular staining and cellular characteristic detection method described in Bray Mass., et al., Nat. Protoc., 11(9):1757-74 (2016), generating measurements for over 1000 different cellular characteristics for each experimental state. The data was normalized and then featurized by principal component analysis.

Pairwise analysis of the IL13 screen against a first plurality of compounds was next performed, as described in Example 1. The first plurality of compounds included 15 known JAK inhibitors and 237 compounds that were not previously known to be JAK inhibitors. Two-way ANOVA on an ordinary least squares linear model was performed on the first 10 principal component of each of the 252 combinations of a baseline state, perturbation state (anti-IL13 siRNA), the drug state (compound), and combination state (anti-IL13 siRNA and compound). As expected, p-values for individual principal components of known JAK inhibitors showed a statistically significant interaction between the JAK inhibitor and the IL13 gene perturbation. An example of some of these p-values is shown in Table 11.

TABLE 11
Statistics for two-way ANOVA of the first 7 principal components, for
interactions between an anti-IL13 siRNA and known JAK inhibitors.

| | P0 | P1 | P2 | P3 | P4 | P5 | P6 | P7 |
|---|---|---|---|---|---|---|---|---|
| REC-0000267 | 8.279425e−01 | 0.435576 | 0.567716 | 1.727986e−04 | 0.650566 | 0.630570 | 0.394450 | 5.846837e−06 |
| REC-0000383 | 3.680806e−05 | 0.000121 | 0.022012 | 8.045158e−01 | 0.076273 | 0.005822 | 0.000004 | 4.431539e−02 |
| REC-0000439 | 4.004581e−01 | 0.809795 | 0.509780 | 5.579580e−02 | 0.067589 | 0.328958 | 0.013203 | 5.159663e−01 |
| REC-0000750 | 3.276757e−18 | 0.021338 | 0.003291 | 3.702388e−07 | 0.000367 | 0.161920 | 0.001294 | 5.276806e−21 |
| REC-0000811 | 4.910029e−01 | 0.112492 | 0.614453 | 1.293135e−01 | 0.054527 | 0.164677 | 0.725670 | 4.540459e−01 |

Next, the p-values for all of the principal components for the known JAK inhibitors were combined, to provide a single test statistic for the significance of the interaction between each JAK inhibitor and the IL13 gene perturbation. As shown in Table 12, 93% (14/15) of the JAK inhibitors give a significantly significant interaction score (combined p<0.05), using this test statistic.

TABLE 12
Combined p-value test statistic from two-way ANOVA
of all principal components, for interactions between
an anti-IL13 siRNA and known JAK inhibitors.

| | combined_pvalue |
|---|---|
| REC-0000333 | 0.000000e+00 |
| REC-0000750 | 0.000000e+00 |
| REC-0001886 | 0.000000e+00 |
| REC-0001076 | 1.110223e−16 |
| REC-0001884 | 3.509030e−10 |
| REC-0002108 | 5.237455e−09 |
| REC-0000267 | 1.637742e−07 |
| REC-0000835 | 3.193006e−07 |
| REC-0000886 | 2.927008e−06 |
| REC-0001556 | 1.339893e−05 |
| REC-0003935 | 3.229571e−03 |
| REC-0000811 | 3.186106e−02 |
| REC-0002000 | 3.534027e−02 |
| REC-0000439 | 3.852483e−02 |
| REC-0001615 | 2.927996e−01 |

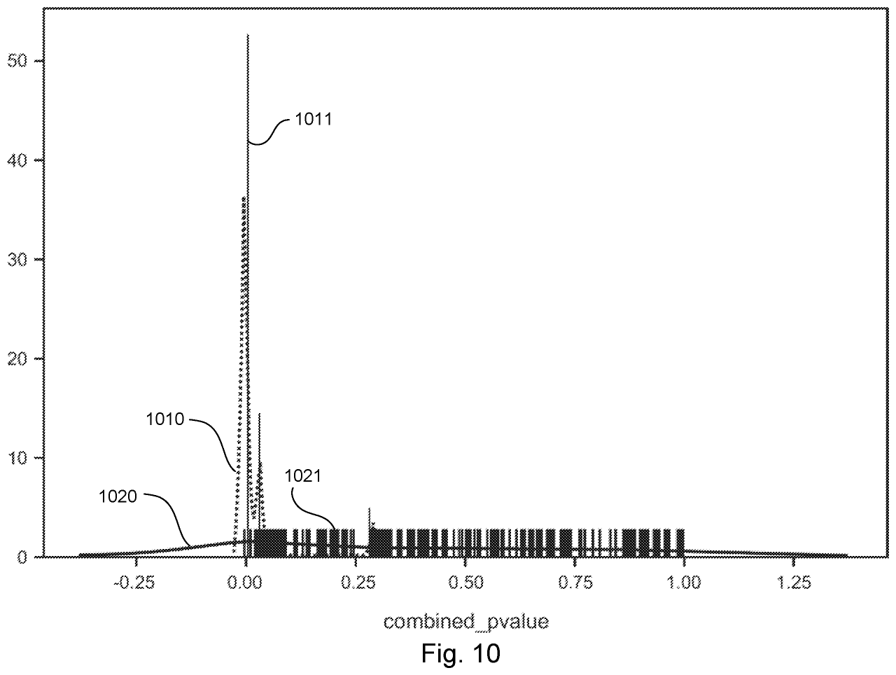

In contrast, a much smaller percentage of compounds that were previously unannotated for JAK inhibition provided a statistically significant test statistic when two-way ANOVA p-values calculated across all principal components were combined. As seen in FIG. 10, a significant enrichment for the known JAK inhibitors occurs when the combined p-value test statistic for each compound (known JAK inhibitors (1010, 1011); unannotated compounds (1020, 1021) is plotted on a rugplot having a logarithmic x-axis. Line 1010 is an average of the data on line 1011. Line 1020 is an average of the date on line 1021.

Pairwise analysis of the IL6 screen against a second plurality of compounds was next performed, as described in Example 1. The second plurality of compounds included 5 known JAK inhibitors and more than 100 compounds that were not previously known to be JAK inhibitors. Two-way ANOVA on an ordinary least squares linear model was performed on the first 10 principal component of each of the combinations of a baseline state, perturbation state (anti-IL6 siRNA), the drug state (compound), and combination state (anti-IL6 siRNA and compound). As expected, p-values for individual principal components of known JAK inhibitors showed a statistically significant interaction between the JAK inhibitor and the IL6 gene perturbation. Next, the p-values for all of the principal components for the known JAK inhibitors were combined, to provide a single test statistic for the significance of the interaction between each JAK inhibitor and the IL6 gene perturbation. As shown in Table 13, 60% (3/5) of the JAK inhibitors give a significantly significant interaction score, using this test statistic.

TABLE 13
Combined p-value test statistic from two-way ANOVA
of all principal components, for interactions between
an anti-IL6 siRNA and known JAK inhibitors.

| | combined_pvalue |
|---|---|
| 0 | |
| REC-0000383 | 0.000000 |
| REC-0000439 | 0.674928 |
| REC-0000750 | 0.000000 |
| REC-0001615 | 0.675070 |
| REC-0002108 | 0.000073 |

While only 60% of the known JAK inhibitors showed a significant interaction, it is worth noting that both of the JAK inhibitors that did show a significant interaction with the IL6 gene perturbation (REC-000439 and REC-0001615) showed the least significant interactions with the IL13 gene perturbation above. This suggests that better enrichment of known JAK inhibitors could be achieved if more JAK inhibitors were tested.

### Succinct Descriptions of Various Aspects

In one aspect of a computer system for determining whether a first cellular perturbation interacts with a second cellular perturbation in one of a specific cellular context and a background, in a cell based assay, the cell based assay comprising a plurality of wells across one or more plates, the computer system comprises: one or more processors; a memory; and one or more programs, wherein the one or more programs are stored in the memory and are configured to be executed by the one or more processors, the one or more programs including instructions for: obtaining a baseline data point for a baseline state, wherein the baseline data point comprises a plurality of dimensions, in a plurality of cellular characteristics, determined across a plurality of baseline aliquots of cells representing the baseline state in corresponding wells, in the plurality of wells, wherein the baseline state comprises a first cellular context; obtaining a perturbation data point for a perturbation state, wherein the perturbation data point comprises the plurality of dimensions, in the plurality of cellular characteristics, determined across a plurality of perturbation aliquots of cells representing the perturbation state in corresponding wells, in the plurality of wells, wherein the perturbation state comprises a first perturbation of the first cellular context in which expression of a gene is perturbed relative to expression of the gene in the baseline state; obtaining a compound data point for a compound state, wherein the compound data point comprises the plurality of dimensions, in the plurality of cellular characteristics, determined across a plurality of compound aliquots of cells representing the compound state in corresponding wells, in the plurality of wells, wherein the compound state comprises a second perturbation of the first cellular context in which the first cellular context is exposed to a compound; obtaining a combination data point for a combination state, wherein the combination data point comprises the plurality of dimensions, in the plurality of cellular characteristics, determined across a corresponding plurality of combination aliquots of cells representing the combination state in corresponding wells, in the plurality of wells, wherein the combination state comprises a third perturbation of the first cellular context in which (i) expression of the gene is perturbed relative to expression of the gene in the baseline state and (ii) the first cellular context is exposed to the compound; applying a dimension reduction model, in turn, to each of the baseline data point, the perturbation data point, the compound data point, and the combination data point to respectively generate a plurality of baseline feature values for the baseline data point, a plurality of perturbation features values for the perturbation data point, a plurality of compound feature values for the compound data point, and a plurality of combination feature values for the combination data point; and determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics.

In some aspects of the computer system, the determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics comprises: determining the first cellular perturbation interacts with the second cellular perturbation when the combination of the gene and the compound has a threshold effect on one or more cellular characteristic in the plurality of cellular characteristics.

In some aspects of computer system, the determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics comprises: determining the first cellular perturbation does not interact with the second cellular perturbation when the combination of the gene and the compound does not have a threshold effect on one or more cellular characteristic in the plurality of cellular characteristics.

In some aspects of the computer system, the first cellular context is an adherent mammalian cell line.

In some aspects of the computer system, expression of the gene is perturbed, in the perturbation and combination states, by introduction of an siRNA targeting the gene into the first cellular context of (i) the plurality of perturbation aliquots of cells representing the perturbation state and (ii) the plurality of combination aliquots of cells representing the combination state.

In some aspects of the computer system, a single species of siRNA targeting the gene is introduced into the first cellular context of (i) each respective perturbation aliquot, in the plurality of perturbation aliquots of cells representing the perturbation state, and (ii) each respective combination aliquot, in the plurality of combination aliquots of cells representing the combination state. In some aspects, a plurality of siRNA targeting the gene is introduced into the first cellular context of (i) each respective perturbation aliquot, in the plurality of perturbation aliquots of cells representing the perturbation state, and (ii) each respective combination aliquot, in the plurality of combination aliquots of cells representing the combination state. In some aspects, a first species of siRNA targeting the gene is introduced into the first cell context of (i) a first respective perturbation aliquot, in the plurality of perturbation aliquots of cells representing the perturbation state, and (ii) a first respective combination aliquot, in the plurality of combination aliquots of cells representing the combination state, and a second species of siRNA targeting the gene is introduced into the first cell context of (i) a second respective perturbation aliquot, in the plurality of perturbation aliquots of cells representing the perturbation state, and (ii) a second respective combination aliquot, in the plurality of combination aliquots of cells representing the combination state.

In some aspects of the above described computer system, expression of the gene is perturbed, in the perturbation and combination states, by introduction of a CRISPR reagent targeting the gene into the first cellular context of (i) the plurality of perturbation aliquots of cells representing the perturbation state and (ii) the plurality of combination aliquots of cells representing the combination state.

In some aspects of the above described computer system, the dimension reduction model is a set of principal components explaining variance across a training dataset comprising measurements of the plurality of cellular characteristics determined across a plurality of experimental states, wherein each experimental state in the plurality of experimental states comprises a cellular context.

In some aspects of the above described computer system, the dimension reduction model makes use of a neural network, wherein: the neural network comprises: an input layer comprising the plurality of dimensions, wherein the input layer receives the baseline data point, perturbation data point, compound data point, or combination data point, and an embedding layer that directly or indirectly receives output from the input layer, wherein the embedding layer is associated with a plurality of weights and, responsive to input of data into the neural network, produces an embedding layer output having fewer dimensions than the plurality of dimensions; and wherein: the plurality of weights was trained against a training dataset comprising measurements of the plurality of cellular characteristics determined across a plurality of reference experimental states using a loss function, wherein each reference experimental state in the plurality of reference experimental states comprises an independent cellular context. In some aspects, the neural network was trained in a supervised fashion. In some aspects, the neural network was trained in an unsupervised fashion.

In some aspects of the above described computer system, the determining comprises performing a statistical hypothesis test against at least the plurality of combination feature values using a null hypothesis that the compound does not interact with the gene. In some aspects, the statistical hypothesis test is a two-way ANOVA performed against each respective combination feature value in the plurality of combination feature values, thereby generating a corresponding p-value for each respective combination feature value in the plurality of combination feature values. Some aspects may further comprise generating a test statistic X² by combining the corresponding p-values for each respective combination feature value in the plurality of combination feature values.

In one aspect of a method for determining whether a first cellular perturbation interacts with a second cellular perturbation in one of a specific cellular context and a background, in a cell based assay, the cell based assay comprising a plurality of wells across one or more plates, the method comprises, at a computer system comprising one or more processors and a memory: obtaining a baseline data point for a baseline state, wherein the baseline data point comprises a plurality of dimensions, in a plurality of cellular characteristics, determined across a plurality of baseline aliquots of cells representing the baseline state in corresponding wells, in the plurality of wells, wherein the baseline state comprises a first cellular context; obtaining a perturbation data point for a perturbation state, wherein the perturbation data point comprises the plurality of dimensions, in the plurality of cellular characteristics, determined across a plurality of perturbation aliquots of cells representing the perturbation state in corresponding wells, in the plurality of wells, wherein the perturbation state comprises a first perturbation of the first cellular context in which expression of a gene is perturbed relative to expression of the gene in the baseline state; obtaining a compound data point for a compound state, wherein the compound data point comprises the plurality of dimensions, in the plurality of cellular characteristics, determined across a plurality of compound aliquots of cells representing the compound state in corresponding wells, in the plurality of wells, wherein the compound state comprises a second perturbation of the first cellular context in which the first cellular context is exposed to a compound; obtaining a combination data point for a combination state, wherein the combination data point comprises the plurality of dimensions, in the plurality of cellular characteristics, determined across a corresponding plurality of combination aliquots of cells representing the combination state in corresponding wells, in the plurality of wells, wherein the combination state comprises a third perturbation of the first cellular context in which (i) expression of the gene is perturbed relative to expression of the gene in the baseline state and (ii) the first cellular context is exposed to the compound; applying a dimension reduction model, in turn, to each of the baseline data point, the perturbation data point, the compound data point, and the combination data point to respectively generate a plurality of baseline feature values for the baseline data point, a plurality of perturbation features values for the perturbation data point, a plurality of compound feature values for the compound data point, and a plurality of combination feature values for the combination data point; and determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics.

With reference to the succinct computer system aspects described above, the various aspects of the method may be described in more detail in a similar manner to like aspects of the computer system.

In one aspect a non-transitory computer readable storage medium includes one or more computer programs embedded therein for determining whether a first cellular perturbation interacts with a second cellular perturbation in one of a specific cellular context and a background, in a cell based assay, the cell based assay comprising a plurality of wells across one or more plates. The one or more computer programs comprise instructions which, when executed by a computer system, cause the computer system to perform a method comprising: obtaining a baseline data point for a baseline state, wherein the baseline data point comprises a plurality of dimensions, in a plurality of cellular characteristics, determined across a plurality of baseline aliquots of cells representing the baseline state in corresponding wells, in the plurality of wells, wherein the baseline state comprises a first cellular context; obtaining a perturbation data point for a perturbation state, wherein the perturbation data point comprises the plurality of dimensions, in the plurality of cellular characteristics, determined across a plurality of perturbation aliquots of cells representing the perturbation state in corresponding wells, in the plurality of wells, wherein the perturbation state comprises a first perturbation of the first cellular context in which expression of a gene is perturbed relative to expression of the gene in the baseline state; obtaining a compound data point for a compound state, wherein the compound data point comprises the plurality of dimensions, in the plurality of cellular characteristics, determined across a plurality of compound aliquots of cells representing the compound state in corresponding wells, in the plurality of wells, wherein the compound state comprises a second perturbation of the first cellular context in which the first cellular context is exposed to a compound; obtaining a combination data point for a combination state, wherein the combination data point comprises the plurality of dimensions, in the plurality of cellular characteristics, determined across a corresponding plurality of combination aliquots of cells representing the combination state in corresponding wells, in the plurality of wells, wherein the combination state comprises a third perturbation of the first cellular context in which (i) expression of the gene is perturbed relative to expression of the gene in the baseline state and (ii) the first cellular context is exposed to the compound; applying a dimension reduction model, in turn, to each of the baseline data point, the perturbation data point, the compound data point, and the combination data point to respectively generate a plurality of baseline feature values for the baseline data point, a plurality of perturbation features values for the perturbation data point, a plurality of compound feature values for the compound data point, and a plurality of combination feature values for the combination data point; and determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics.

With reference to the succinct computer system aspects described above, the various aspects of the non-transitory computer readable medium may be described in more detail in a similar manner to like aspects of the computer system.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The described embodiments can be implemented as a computer program product that comprises a computer program mechanism embedded in a nontransitory computer readable storage medium. For instance, the computer program product could contain the program modules shown and/or described in any combination of FIGS. 1A-8D. These program modules can be stored on a CD-ROM, DVD, magnetic disk storage product, USB key, or any other non-transitory computer readable data or program storage product.

Many modifications and variations of the described embodiments can be made without departing from the spirit and scope, as will be apparent to those skilled in the art. The specific embodiments described herein are offered by way of example only. The embodiments were chosen and described in order to best explain principles and practical applications, to thereby enable others skilled in the art to best utilize the various embodiments with various modifications as are suited to the particular use contemplated. The described embodiments are to be limited only by the terms of the appended claims, along with the full scope of equivalents to which such claims are entitled.

What is claimed is:

1. A computer system for determining whether a first cellular perturbation interacts with a second cellular perturbation in one of a specific cellular context and a background, in a cell based assay, the cell based assay comprising a plurality of wells across one or more plates, the computer system comprising:
one or more processors;
a memory; and
one or more programs, wherein the one or more programs are stored in the memory and are configured to be executed by the one or more processors, the one or more programs including instructions for:
obtaining a baseline data point for a baseline state, wherein the baseline data point comprises a plurality of dimensions, in a plurality of cellular characteristics, determined across a plurality of baseline aliquots of cells representing the baseline state in corresponding wells, in the plurality of wells, wherein the baseline state comprises a first cellular context;
obtaining a perturbation data point for a perturbation state, wherein the perturbation data point comprises the plurality of dimensions, in the plurality of cellular characteristics, determined across a plurality of perturbation aliquots of cells representing the perturbation state in corresponding wells, in the plurality of wells, wherein the perturbation state comprises a first perturbation of the first cellular context in which expression of a gene is perturbed relative to expression of the gene in the baseline state;
obtaining a compound data point for a compound state, wherein the compound data point comprises the plurality of dimensions, in the plurality of cellular characteristics, determined across a plurality of compound aliquots of cells representing the compound state in corresponding wells, in the plurality of wells, wherein the compound state comprises a second perturbation of the first cellular context in which the first cellular context is exposed to a compound;
obtaining a combination data point for a combination state, wherein the combination data point comprises the plurality of dimensions, in the plurality of cellular characteristics, determined across a corresponding plurality of combination aliquots of cells representing the combination state in corresponding wells, in the plurality of wells, wherein the combination state comprises a third perturbation of the first cellular context in which (i) expression of the gene is perturbed relative to expression of the gene in the baseline state and (ii) the first cellular context is exposed to the compound;
applying a dimension reduction model, in turn, to each of the baseline data point, the perturbation data point, the compound data point, and the combination data point to respectively generate a plurality of baseline feature values for the baseline data point, a plurality of perturbation features values for the perturbation data point, a plurality of compound feature values for the compound data point, and a plurality of combination feature values for the combination data point; and
determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics.

2. The computer system of claim 1, wherein the determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics comprises:
determining the first cellular perturbation interacts with the second cellular perturbation when the combination of the gene and the compound has a threshold effect on one or more cellular characteristic in the plurality of cellular characteristics.

3. The computer system of claim 1, wherein the determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics comprises:
determining the first cellular perturbation does not interact with the second cellular perturbation when the combination of the gene and the compound does not have a threshold effect on one or more cellular characteristic in the plurality of cellular characteristics.

4. The computer system of claim 1, wherein the first cellular context is an adherent mammalian cell line.

5. The computer system of claim 1, wherein expression of the gene is perturbed, in the perturbation and combination states, by introduction of an siRNA targeting the gene into the first cellular context of (i) the plurality of perturbation aliquots of cells representing the perturbation state and (ii) the plurality of combination aliquots of cells representing the combination state.

6. The computer system of claim 5, wherein a single species of siRNA targeting the gene is introduced into the first cellular context of (i) each respective perturbation aliquot, in the plurality of perturbation aliquots of cells representing the perturbation state, and (ii) each respective combination aliquot, in the plurality of combination aliquots of cells representing the combination state.

7. The computer system of claim 5, wherein a plurality of siRNA targeting the gene is introduced into the first cellular context of (i) each respective perturbation aliquot, in the plurality of perturbation aliquots of cells representing the perturbation state, and (ii) each respective combination aliquot, in the plurality of combination aliquots of cells representing the combination state.

8. The computer system of claim 5, wherein a first species of siRNA targeting the gene is introduced into the first cell context of (i) a first respective perturbation aliquot, in the plurality of perturbation aliquots of cells representing the perturbation state, and (ii) a first respective combination aliquot, in the plurality of combination aliquots of cells representing the combination state, and a second species of siRNA targeting the gene is introduced into the first cell context of (i) a second respective perturbation aliquot, in the plurality of perturbation aliquots of cells representing the perturbation state, and (ii) a second respective combination aliquot, in the plurality of combination aliquots of cells representing the combination state.

9. The computer system of claim 1, wherein expression of the gene is perturbed, in the perturbation and combination states, by introduction of a CRISPR reagent targeting the gene into the first cellular context of (i) the plurality of perturbation aliquots of cells representing the perturbation state and (ii) the plurality of combination aliquots of cells representing the combination state.

10. The computer system of claim 1, wherein the dimension reduction model is a set of principal components explaining variance across a training dataset comprising measurements of the plurality of cellular characteristics determined across a plurality of experimental states, wherein each experimental state in the plurality of experimental states comprises a cellular context.

11. The computer system of claim 1, wherein the dimension reduction model makes use of a neural network, wherein:
the neural network comprises:
an input layer comprising the plurality of dimensions, wherein the input layer receives the baseline data point, perturbation data point, compound data point, or combination data point, and
an embedding layer that directly or indirectly receives output from the input layer, wherein the embedding layer is associated with a plurality of weights and, responsive to input of data into the neural network, produces an embedding layer output having fewer dimensions than the plurality of dimensions; and
wherein:
the plurality of weights was trained against a training dataset comprising measurements of the plurality of cellular characteristics determined across a plurality of reference experimental states using a loss function, wherein each reference experimental state in the plurality of reference experimental states comprises an independent cellular context.

12. The computer system of claim 11, wherein the neural network was trained in a supervised fashion.

13. The computer system of claim 11, wherein the neural network was trained in an unsupervised fashion.

14. The computer system of claim 1, wherein the determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics comprises:
performing a statistical hypothesis test against at least the plurality of combination feature values using a null hypothesis that the compound does not interact with the gene.

15. The computer system of claim 14, wherein the statistical hypothesis test is a two-way ANOVA performed against each respective combination feature value in the plurality of combination feature values, thereby generating a corresponding p-value for each respective combination feature value in the plurality of combination feature values.

16. The computer system of claim 15, further comprising generating a test statistic X′ by combining the corresponding p-values for each respective combination feature value in the plurality of combination feature values.

17. A method for determining whether a first cellular perturbation interacts with a second cellular perturbation in one of a specific cellular context and a background, in a cell based assay, the cell based assay comprising a plurality of wells across one or more plates, the method comprising, at a computer system comprising one or more processors and a memory:
obtaining a baseline data point for a baseline state, wherein the baseline data point comprises a plurality of dimensions, in a plurality of cellular characteristics, determined across a plurality of baseline aliquots of cells representing the baseline state in corresponding wells, in the plurality of wells, wherein the baseline state comprises a first cellular context;
obtaining a perturbation data point for a perturbation state, wherein the perturbation data point comprises the plurality of dimensions, in the plurality of cellular characteristics, determined across a plurality of perturbation aliquots of cells representing the perturbation state in corresponding wells, in the plurality of wells, wherein the perturbation state comprises a first perturbation of the first cellular context in which expression of a gene is perturbed relative to expression of the gene in the baseline state;
obtaining a compound data point for a compound state, wherein the compound data point comprises the plurality of dimensions, in the plurality of cellular characteristics, determined across a plurality of compound aliquots of cells representing the compound state in corresponding wells, in the plurality of wells, wherein the compound state comprises a second perturbation of the first cellular context in which the first cellular context is exposed to a compound;
obtaining a combination data point for a combination state, wherein the combination data point comprises the plurality of dimensions, in the plurality of cellular characteristics, determined across a corresponding plurality of combination aliquots of cells representing the combination state in corresponding wells, in the plurality of wells, wherein the combination state comprises a third perturbation of the first cellular context in which (i) expression of the gene is perturbed relative to expression of the gene in the baseline state and (ii) the first cellular context is exposed to the compound;
applying a dimension reduction model, in turn, to each of the baseline data point, the perturbation data point, the compound data point, and the combination data point to respectively generate a plurality of baseline feature values for the baseline data point, a plurality of perturbation features values for the perturbation data point, a plurality of compound feature values for the compound data point, and a plurality of combination feature values for the combination data point; and
determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics.

18. The method as recited in claim 17, wherein the determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics comprises:
determining the first cellular perturbation interacts with the second cellular perturbation when the combination of the gene and the compound has a threshold effect on one or more cellular characteristic in the plurality of cellular characteristics.

19. The method as recited in claim 17, wherein the determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics comprises:
determining the first cellular perturbation does not interact with the second cellular perturbation when the combination of the gene and the compound does not have a threshold effect on one or more cellular characteristic in the plurality of cellular characteristics.

20. A non-transitory computer readable storage medium having one or more computer programs embedded therein for determining whether a first cellular perturbation interacts with a second cellular perturbation in one of a specific cellular context and a background, in a cell based assay, the cell based assay comprising a plurality of wells across one or more plates, the one or more computer programs comprising instructions which, when executed by a computer system, cause the computer system to perform a method comprising:
obtaining a baseline data point for a baseline state, wherein the baseline data point comprises a plurality of dimensions, in a plurality of cellular characteristics, determined across a plurality of baseline aliquots of cells representing the baseline state in corresponding wells, in the plurality of wells, wherein the baseline state comprises a first cellular context;
obtaining a perturbation data point for a perturbation state, wherein the perturbation data point comprises the plurality of dimensions, in the plurality of cellular characteristics, determined across a plurality of perturbation aliquots of cells representing the perturbation state in corresponding wells, in the plurality of wells, wherein the perturbation state comprises a first perturbation of the first cellular context in which expression of a gene is perturbed relative to expression of the gene in the baseline state;
obtaining a compound data point for a compound state, wherein the compound data point comprises the plurality of dimensions, in the plurality of cellular characteristics, determined across a plurality of compound aliquots of cells representing the compound state in corresponding wells, in the plurality of wells, wherein the compound state comprises a second perturbation of the first cellular context in which the first cellular context is exposed to a compound;
obtaining a combination data point for a combination state, wherein the combination data point comprises the plurality of dimensions, in the plurality of cellular characteristics, determined across a corresponding plurality of combination aliquots of cells representing the combination state in corresponding wells, in the plurality of wells, wherein the combination state comprises a third perturbation of the first cellular context in which (i) expression of the gene is perturbed relative to expression of the gene in the baseline state and (ii) the first cellular context is exposed to the compound;
applying a dimension reduction model, in turn, to each of the baseline data point, the perturbation data point, the compound data point, and the combination data point to respectively generate a plurality of baseline feature values for the baseline data point, a plurality of perturbation features values for the perturbation data point, a plurality of compound feature values for the compound data point, and a plurality of combination feature values for the combination data point; and
determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics.

21. The non-transitory computer readable storage medium as recited in claim 20, wherein the determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics comprises:
determining the first cellular perturbation interacts with the second cellular perturbation when the combination of the gene and the compound has a threshold effect on one or more cellular characteristic in the plurality of cellular characteristics.

22. The non-transitory computer readable storage medium as recited in claim 20, wherein the determining whether a first cellular perturbation interacts with a second cellular perturbation in one of a specific cellular context and a background by using the plurality of baseline feature values, the plurality of perturbation feature values, the plurality of compound feature values, and the plurality of combination feature values to resolve whether the interaction of the first cellular perturbation with the second cellular perturbation has a threshold interaction effect on one or more cellular characteristic in the plurality of cellular characteristics comprises:
determining the first cellular perturbation does not interact with the second cellular perturbation when the combination of the gene and the compound does not have a threshold effect on one or more cellular characteristic in the plurality of cellular characteristics.
